SEQUENCE LISTING (22 sequences)

SEQ ID NO: 1 - DNA, 1923 bp, Homo sapiens
  source 1..1923: mol_type = genomic DNA
atggcggcgggggggacagggggcctgcgggaggagcagcgctatgggctgtcgtgcggacggctggggcaggacaacatcaccgtactgcatgtgaagctcaccgagacggcgatccgggcgctcgagacttaccagagccacaagaatttaattccttttcgaccttcaatccagttccaaggactccacgggcttgtcaaaattcccaaaaatgatcccctcaatgaagttcataactttaacttttatttgtcaaatgtgggcaaagacaaccctcagggcagctttgactgcatccagcaaacattctccagctctggagcctcccagctcaattgcctgggatttatacaagataaaattacagtgtgtgcaacaaacgactcgtatcagatgacacgagaaagaatgacccaggcagaggaggaatcccgcaaccgaagcacaaaagttatcaaacccggtggaccatatgtagggaaaagagtgcaaattcggaaagcacctcaagctgtttcagatacagttcctgagaggaaaaggtcaacccccatgaaccctgcaaatacaattcgaaagacacatagcagcagcaccatctctcagaggccatacagggacagggtgattcacttactggccctgaaggcctacaagaaaccggagctacttgctagactccagaaagatggtgtcaatcaaaaagacaagaactccctgggagcaattctgcaacaggtagccaatctgaattctaaggacctctcatataccttaaaggattatgtttttaaagagcttcaaagagactggcctggatacagtgaaatagacagacggtcattggagtcagtgctctctagaaaactaaatccgtctcagaatgctacaggcaccagccgttcagaatctcctgtatgttctagtagagatgctgtatcttctcctcagaaacggcttttggattcagagtttattgatcctttaatgaataaaaaagcccgaatatctcacctgacgaacagagtaccaccaacactaaatggtcatttgaatcccaccagtgaaaaatcggctgcaggcctcccactgccccctgcggctgctgccatccccacccctccaccgctgccttcaacctatctgcccatctcacatcctcctcagattgtaaattctaactccaactcccctagcactccagaaggccgggggactcaagacctacctgttgacagttttagtcaaaacgatagtatctatgaggaccagcaagacaaatatacctctaggacttctctggaaaccttaccccctggttccgttctactaaagtgtccaaagcctatggaagaaaaccattcaatgtctcacaaaaagtccaaaaagaagtctaaaaaacataaggaaaaggaccaaataaaaaagcacgacattgagactattgaggaaaaggaggaagatcttaagagagaagaggaaattgccaagctaaataactccagtccaaattccagtggaggagttaaagaggattgcactgcctccatggaaccttcagcaattgaactcccagattatttgataaaatatatcgctatcgtctcctatgagcaacgccagaattataaggatgacttcaatgcagagtatgatgagtacagagctttgcatgccaggatggagactgtagctagaagatttatcaaactagatgcacaaagaaagcgcctttctccaggctcaaaagagtatcagaatgttcatgaagaagtcttacaagaatatcagaagataaagcagtctagtcccaattaccatgaagaaaaatacagatgtgaatatcttcataacaagctggctcacatcaaaaggctaataggtgaatttgaccaacagcaagcagagtcatggtcctag

SEQ ID NO: 2 - DNA, 1194 bp, Homo sapiens
  source 1..1194: mol_type = genomic DNA
atggaggagctccaggagcctctgagaggacagctccggctctgcttcacgcaagctgcccggactagcctcttactgctcaggctcaacgacgctgccctgcgggcgctgcaagagtgtcagcggcaacaggtacggccggtgattgctttccaaggccaccgagggtatctgagactcccaggccctggttggtcctgcctcttctccttcatagtgtcccagtgttgtcaggagggcgctggtggtagcttggaccttgtgtgccaacgcttcctcaggtctgggcctaacagcctccactgcctgggctcactcagggagcgcctcattatttgggcagccatggattctatcccagccccatcatcagttcagggacacaacctgactgaagatgccagacatcctgagagttggcagaacacaggaggctattctgaaggagatgcagtatcacagccacagatggcactagaggaggtgtcagtgtcagatccactggcaagcaaccaaggacagtcactcccaggatcctcaagggagcacatggcacagtgggaagtgagaagccagacccatgttccaaacagagaacctgttcaggcactgccttcctctgccagccggaaacgtctggacaagaaacgttcagtgcctgtagccactgtagaactggaagaaaagaggttcagaactctgcctttagtgccaagccccctacaaggcctgaccaatcaggatttacaagagggagaagattgggagcaagaagatgaggacatggaccccagattagaacacagttcctcagttcaagaagattctgaatccccaagtcctgaagatataccagactacctcctgcaatacagggccatccacagtgcagaacagcaacatgcctatgagcaggactttgagacagattatgctgaataccgcatcctgcatgcccgtgttgggactgcaagccaaaggttcatagagctgggagcagagattaaaagagttcggcgaggaactccagaatacaaggtcctggaagacaagataatccaggaatataaaaagttcaggaagcagtacccaagttacagagaagaaaagcgtcgctgtgagtaccttcaccagaaattgtcccacattaaaggtctcatcctggagtttgaggaaaagaacaggggcagctga

SEQ ID NO: 3 - DNA, 807 bp, Homo sapiens
  source 1..807: mol_type = genomic DNA
atgaatgggaccgcaaacccgctgctggaccgcgaggaacattgcctgaggctcggggagagcttcgagaagcggccgcgggcctccttccacactattcgttatgattttaaaccagcatctatagacacttcctgtgaaggagagcttcaagttggcaaaggagatgaagtcacaattacactgccacatatccctggatccacaccacccatgactgtgttcaaggggaacaaacggccttaccagaaagactgtgtgcttattattaatcatgacactggtgaatatgtgctggaaaaactcagtagcagcattcaggtgaagaaaacaagagctgagggcagcagtaaaatccaggcccgaatggaacagcagcccactcgtcctccacagacgtcacagccaccaccacctccaccacctatgccattcagagctccaacgaagcctccagttggacccaaaacttctcccttgaaagataacccgtcacctgaacctcagttggatgacatcaaaagagagctgagggctgaagttgacattattgaacaaatgagcagcagcagtgggagcagctcttcagactctgagagctcttcgggaagtgatgacgatagctccagcagtggaggcgaggacaatggcccagcctctcctccgcagccttcacaccagcagccctacaacagtaggcctgccgttgccaatggaaccagccggccacaaggaagcaaccagctcatgaacaccctcagaaatgacttgcagttgagtgagtctggcagtgacagtgatgactag

SEQ ID NO: 4 - DNA, 783 bp, Homo sapiens
  source 1..783: mol_type = genomic DNA
atgaatagcgcagcgggattctcacacctagaccgtcgcgagcgggttctcaagttaggggagagtttcgagaagcagccgcgctgcgccttccacactgtgcgctatgacttcaaacctgcttctattgacacttcttctgaaggataccttgaggttggtgaaggtgaacaggtgaccataactctgccaaatatagaaggttcaactccaccagtaactgttttcaaaggttcaaaaaaaccttacttaaaagaatgcattttgattattaaccatgatactggagaatgtcggctagaaaaactcagcagcaacatcactgtaaaaaaaacaagagttgaaggaagcagtaaaattcagtatcgtaaagaacaacagcaacaacaaatgtggaattcagccaggactcccaatcttgtaaaacattctccatctgaagataagatgtccccagcatctccaatagatgatatcgaaagagaactgaaggcagaagctagtctaatggaccagatgagtagttgtgatagttcatcagattccaaaagttcatcatcttcaagtagtgaggatagttctagtgactcagaagatgaagattgcaaatcctctacttctgatacagggaattgtgtctcaggacatcctaccatgacacagtacaggattcctgatatagatgccagtcataatagatttcgagacaacagtggccttctgatgaatactttaagaaatgatttgcagctgagtgaatcaggaagtgacagtgatgactga

SEQ ID NO: 5 - DNA, 1680 bp, Homo sapiens
  source 1..1680: mol_type = genomic DNA
atggacaatcagtgcaccgtccaggtgaggttagagctggggcatcgcgcccaactgcgcaagaagcccaccacggaggggttcactcacgactggatggtgtttgtccgcggccccgagcaatgtgacatccagcacttcgtggagaaggtggtcttctggctgcacgacagcttccccaagcccagacgcgtgtgcaaggagcccccctacaaagtagaggagtcggggtacgctggcttcatcatgcccatcgaggtgcacttcaaaaacaaggaggagccgaggaaggtctgcttcacctacgacctgttcctgaacctggaaggcaacccgcccgtgaaccacctgcgctgcgagaagctcaccttcaacaaccccaccacggagttccggtacaagctcctgcgggccggcggggtgatggtaatgcccgaaggagcagacacggtgtccaggcccagtcccgactaccccatgttacccacaattccactctctgccttctctgaccccaagaagaccaaaccatcccacggctccaaggacgccaacaaggagagcagcaagacctccaagccacacaaggtgaccaaggagcaccgggagcgcccccgcaaagactccgagagcaagagctcctccaaggagctggagcgtgagcaggccaaaagctccaaggacacctcgcggaagctgggcgagggccggctgcccaaggaggagaaggcgccaccgcccaaggctgccttcaaggaacccaagatggccctgaaagagaccaagctggaaagcacgtcccccaaccctgggcccccacccccacccccacccccaccccgggcttccagcaagcggccggccaccgccgactcgccaaagcccagcgccaagaagcagaagaagagcagctcgaaggggtcccggagtgctccaggcacctcgccccgcacctcctcctcctcctccttctcggacaagaagccggccaaggacaagagcagcaccagaggggagaaggtgaaggccgagagtgagccccgggaggccaaaaaggccctggaggtggaggagtccaactcagaggacgaggcctccttcaagtccgagtctgcccagtcaagcccgtccaactccagctccagctcagactccagctcagactcagacttcgagccatcccagaaccacagccaaggacccctgcgctccatggtggaggacctgcagtccgaggagtccgacgaggacgactcttcgtcaggcgaggaggctgccggcaagaccaacccggggagggactccaggttgagcttcagcgacagcgagagtgacaacagcgccgactcctccctgcccagccgtgagcccccacccccccagaagccacccccgcccaacagcaaggtgtcaggccggaggagccccgagtcctgcagcaagcctgagaagatcctcaagaagggcacctacgacaaggcctacacggatgagctggtggagctacaccggaggctgatggcgctgcgggagcgcaacgtgctgcagcagattgtgaatctgatcgaggagactggccacttcaatgtcaccaacaccaccttcgacttcgacctcttctccctggacgagaccaccgtgcgcaaactgcagagctgcctggaggccgtggccacatga

SEQ ID NO: 6 - DNA, 1707 bp, Homo sapiens
  source 1..1707: mol_type = genomic DNA
atggctagctcgtgttccgtgcaggtgaagctggagctggggcaccgcgcccaggtgaggaaaaaacccaccgtggagggcttcacccacgactggatggtgttcgtacgcggtccggagcacagtaacatacagcactttgtggagaaagtcgtcttccacttgcacgaaagctttcctaggccaaaaagagtgtgcaaagatccaccttacaaagtagaagaatctgggtatgctggtttcattttgccaattgaagtttattttaaaaacaaggaagaacctaggaaagtccgctttgattatgacttattcctgcatcttgaaggccatccaccagtgaatcacctccgctgtgaaaagctaactttcaacaaccccacagaggactttaggagaaagttgctgaaggcaggaggggaccctaataggagtattcataccagcagcagcagcagcagcagcagtagcagcagcagcagcagcagcagcagcagcagtagcagcagcagcagcagcagcagcagcagcagtagcagcagcagtagcagcagcagcagcagcagtagtaccagtttttcaaagcctcacaaattaatgaaggagcacaaggaaaaaccttctaaagactccagagaacataaaagtgccttcaaagaaccttccagggatcacaacaaatcttccaaagaatcctctaagaaacccaaagaaaataaaccactgaaagaagagaaaatagttcctaagatggccttcaaggaacctaaacccatgtcaaaagagccaaaaccagatagtaacttactcaccatcaccagtggacaagataagaaggctcctagtaaaaggccgcccatttcagattctgaagaactctcagccaaaaaaaggaaaaagagtagctcagaggctttatttaaaagtttttctagcgcaccaccactgatactcacttgttctgctgacaaaaaacagataaaagataaatctcatgtcaagatgggaaaggtcaaaattgaaagtgagacatcagagaagaagaaatcaacgttaccgccatttgatgatattgtggatcccaatgattcagatgtggaggagaatatatcctctaaatctgattctgaacaacccagtcctgccagctccagctccagctccagctccagcttcacaccatcccagaccaggcaacaaggtcctttgaggtctataatgaaagatctgcattctgatgacaatgaggaggaatcagatgaagtggaggataacgacaatgactctgaaatggagaggcctgtaaatagaggaggcagccgaagtcgcagagttagcttaagtgatggcagcgatagtgaaagcagttctgcttcttcacccctacatcacgaacctccaccacccttactaaaaaccaacaacaaccagattcttgaagtgaaaagtccaataaagcaaagcaaatcagataagcaaataaagaatggtgaatgtgacaaggcatacctagatgaactggtagagcttcacagaaggttaatgacattgagagaaagacacattctgcagcagatcgtgaaccttatagaagaaactggacactttcatatcacaaacacaacatttgattttgatctttgctcgctggacaaaaccacagtccgtaaactacagagttacctggaaacatctggaacatcctga

SEQ ID NO: 7 - DNA, 3633 bp, Homo sapiens
  source 1..3633: mol_type = genomic DNA
atggcagcccagtcaagtttgtacaatgacgacagaaacctgcttcgaattagagagaaggaaagacgcaaccaggaagcccaccaagagaaagaggcatttcctgaaaagattcccctttttggagagccctacaagacagcaaaaggtgatgagctgtctagtcgaatacagaacatgttgggaaactacgaagaagtgaaggagttccttagtactaagtctcacactcatcgcctggatgcttctgaaaataggttgggaaagccgaaatatcctttaattcctgacaaagggagcagcattccatccagctccttccacactagtgtccaccaccagtccattcacactcctgcgtctggaccactttctgttggcaacattagccacaatccaaagatggcgcagccaagaactgaaccaatgccaagtctccatgccaaaagctgcggcccaccggacagccagcacctgacccaggatcgccttggtcaggaggggttcggctctagtcatcacaagaaaggtgaccgaagagctgacggagaccactgtgcttcggtgacagattcggctccagagagggagctttctcccttaatctctttgccttccccagttccccctttgtcacctatacattccaaccagcaaactcttccccggacgcaaggaagcagcaaggttcatggcagcagcaataacagtaaaggctattgcccagccaaatctcccaaggacctagcagtgaaagtccatgataaagagacccctcaagacagtttggtggcccctgcccagccgccttctcagacatttccacctccctccctcccctcaaaaagtgttgcaatgcagcagaagcccacggcttatgtccggcccatggatggtcaagatcaggcccctagtgaatcccctgaactgaaaccactgccggaggactatcgacagcagacctttgaaaaaacagacttgaaagtgcctgccaaagccaagctcaccaaactgaagatgccttctcagtcagttgagcagacctactccaatgaagtccattgtgttgaagagattctgaaggaaatgacccattcatggccgcctcctttgacagcaatacatacgcctagtacagctgagccatccaagtttcctttccctacaaaggactctcagcatgtcagttctgtaacccaaaaccaaaaacaatatgatacatcttcaaaaactcactcaaattctcagcaaggaacgtcatccatgctcgaagacgaccttcagctcagtgacagtgaggacagtgacagtgaacaaaccccagagaagcctccctcctcatctgcacctccaagtgctccacagtcccttccagaaccagtggcatcagcacattccagcagtgcagagtcagaaagcaccagtgactcagacagttcctcagactcagagagcgagagcagttcaagtgacagcgaagaaaatgagcccctagaaaccccagctccggagcctgagcctccaacaacaaacaaatggcagctggacaactggctgaccaaagtcagccagccagctgcgccaccagagggccccaggagcacagagcccccacggcggcacccagagagtaagggcagcagcgacagtgccacgagtcaggagcattctgaatccaaagatcctccccctaaaagctccagcaaagccccccgggccccacccgaagccccccaccccggaaagaggagctgtcagaagtctccggcacagcaggagcccccacaaaggcaaaccgttggaaccaaacaacccaaaaaacctgtcaaggcctctgcccgggcaggttcacggaccagcctgcagggggaaagggagccagggcttcttccctatggctcccgagaccagacttccaaagacaagcccaaggtgaagacgaaaggacggccccgggccgcagcaagcaacgaacccaagccagcagtgcccccctccagtgagaagaagaagcacaagagctccctccctgccccctctaaggctctctcaggcccagaacccgcgaaggacaatgtggaggacaggacccctgagcactttgctcttgttcccctgactgagagccagggcccaccccacagtggcagcggcagcaggactagtggctgccgccaagccgtggtggtccaggaggacagccgcaaagacagactcccattgcctttgagagacaccaagctgctctcaccgctcagggacactcctcccccacaaagcttgatggtgaagatcaccctagacctgctctctcggataccccagcctcccgggaaggggagccgccagaggaaagcagaagataaacagccgcccgcagggaagaagcacagctctgagaagaggagctcagacagctcaagcaagttggccaaaaagagaaagggtgaagcagaaagagactgtgataacaagaaaatcagactggagaaggaaatcaaatcacagtcatcttcatcttcatcctcccacaaagaatcttctaaaacaaagccctccaggccctcctcacagtcctcaaagaaggaaatgctccccccgccacccgtgtcctcgtcctcccagaagccagccaagcctgcacttaagaggtcaaggcgggaagcagacacctgtggccaggaccctcccaaaagtgccagcagtaccaagagcaaccacaaagactcttccattcccaagcagagaagagtagaggggaagggctccagaagctcctcggagcacaagggttcttccggagatactgcaaatccttttccagtgccttctttgccaaatggtaactctaaaccagggaagcctcaagtgaagtttgacaaacaacaagcagaccttcacatgagggaggcaaaaaagatgaagcagaaagcagagttaatgacggacagggttggaaaggcttttaagtacctggaagccgtcttgtccttcattgagtgcggaattgccacagagtctgaaagccagtcatccaagtcagcttactctgtctactcagaaactgtagatctcattaaattcataatgtcattaaaatccttctcagatgccacagcgccaacacaagagaaaatatttgctgttttatgcatgcgttgccagtccattttgaacatggcgatgtttcgttgtaaaaaagacatagcaataaagtattctcgtactcttaataaacacttcgagagttcttccaaagtcgcccaggcaccttctccatgcattgcaagcacaggcacaccatcccctctttccccaatgccttctcctgccagctccgtagggtcccagtcaagtgctggcagtgtggggagcagtggggtggctgccactatcagcaccccagtcaccatccagaatatgacatcttcctatgtcaccatcacatcccatgttcttaccgcctttgacctttgggaacaggccgaggccctcacgaggaagaataaagaattctttgctcggctcagcacaaatgtgtgcaccttggccctcaacagcagtttggtggacctggtgcactatacacgacagggttttcagcagctacaagaattaaccaaaacaccttaa

SEQ ID NO: 8 - DNA, 3492 bp, Homo sapiens
  source 1..3492: mol_type = genomic DNA
atgaaccgtgaagaccggaatgtgctgcgtatgaaagaacgggaaaggcggaatcaggaaattcagcagggcgaagacgccttcccacctagctctcctctctttgcagagccatacaaagttactagcaaagaagataagttatcaagtcgtattcagagtatgcttggaaactacgatgaaatgaaggatttcataggagacagatctataccaaagcttgttgcaattcccaagcctacagtaccaccatcagcagatgaaaaatctaacccaaatttctttgaacagagacatggaggctctcatcagagtagcaaatggactccagtaggacccgcacccagcacttctcagtctcagaaacggtcctcaggcttacagagtggacatagtagccagcggaccagcgcaggtagcagtagtggcactaacagtagtggtcagaggcacgaccgtgagtcatataacaatagtgggagcagtagccggaaaaaaggccagcatggatcagaacactccaaatcacgttcttccagccctggaaaaccccaggctgtttcttcattaaactctagtcattccaggtctcatgggaatgatcaccatagcaaggaacatcaacgctccaaatcacctcgggaccctgatgcaaactgggattctccttcccgtgtacctttttcaagtgggcagcactcaactcaatctttcccaccctcattgatgtcaaagtccaattcaatgttacagaaacccactgcctatgtgcggcccatggacggacaggagtccatggaaccaaagctgtcctctgagcactacagcagccaatcccatggcaacagcatgactgagctgaagcccagcagcaaagcacatctcaccaagctgaaaataccttcccaaccactggatgcatcagcttctggtgatgtgagctgtgtggatgaaatcctaaaagagatgacgcattcatggcctccccctctaacggctattcatacaccatgcaaaacagaaccttccaaatttccttttccaactaaggagtctcagcagtccaattttggcactggagaacaaaaaagatataatccttctaaaacttcaaatgggcaccagtctaaatctatgttaaaagatgacttaaaactaagcagcagtgaagacagtgatggggaacaggattgtgataagacaatgccgaggagtacaccaggaagtaactctgaaccttcacaccataatagtgaaggagcagataactccagggatgattctagtagccacagtggatctgaaagcagctctggatctgactcagagagtgaaagtagttccagtgacagtgaggcaaatgagccatcccagagtgcatctcccgagcctgaacccccgccaacaaacaaatggcaacttgataattggctgaataaagtgaacccacataaagtgtcacccgcctcttcagtggacagtaacatcccatcatctcaaggctacaaaaaggaaggccgagagcagggcactgggaatagctacactgatacaagtggacctaaagaaacgagttccgctactccgggacgagactccaaaaccatccaaaagggatcagaaagtgggcgtgggaggcagaaatctcctgcacagagtgacagcacaacacagagaagaactgtaggcaaaaaacaacccaaaaaggctgagaaggcagctgctgaagagcctcgtggaggcctgaagatagaaagtgaaacccctgtagacttggctagcagcatgccctccagcagacacaaagcagccaccaaaggctcaaggaaacccaatataaagaaggagtctaagtcttcccctcgacctacagcagagaaaaagaaatataagtcaacaagtaaatcttcccagaaatcaagggaaatcatagaaacagatacctcatcctcagattcagatgaaagtgagagccttcctccttcctcacaaactcctaagtaccccgagagcaataggactcctgttaaaccctcctcagtggaggaagaagatagcttttttcggcaacgaatgttctctcctatggaagagaaggaacttctttcacccctcagtgagcctgatgacaggtacccacttattgtgaagattgacctgaatcttttgactagaataccaggaaagccttacaaagaaacagagccgcccaagggggaaaagaaaaatgtgccagaaaagcacacgagagaggctcagaaacaagcctcagaaaaagtttccaacaaaggcaagaggaagcataagaatgaagatgataaccgagccagtgagagcaagaaacccaaaacggaggacaagaattcagcaggccataagccatccagcaacagagagtcatctaagcagagtgctgcaaaagaaaaggatttgttgccttctcccgctgggcctgttccttcaaaagatccaaaaacagagcatggctctcggaagaggactattagtcagtcttcttccttaaagtcaagcagtaacagcaacaaggagacgagtggcagcagcaaaaacagttcctccacatcaaagcagaagaagaccgaagggaagacttccagtagctccaaggaggttaaggaaaaggctccaagtagctcctctaactgtcctccatctgcaccaactcttgattcttctaagcctcggagaacaaagcttgtctttgatgacagaaattattcagcagaccattatttacaagaagcaaaaaagctaaagcacaatgcagatgcattgtctgataggtttgagaaagctgtatactatcttgatgctgtggtatctttcattgaatgtgggaatgcattagagaagaatgctcaggaatccaaatccccattccctatgtattcagagacggtggatctcatcaaatacactatgaagctaaagaattacttggcaccagatgctacagctgcagataaacgactcacagtactttgcctgcgatgcgagtctttgctgtacctgaggctgttcaaactgaagaaggaaaatgctctgaagtactcaaagacactgacagagcacctgaagaattcttataataattctcaagcaccatcgcctggcttgggaagcaaagctgtggggatgccttcccctgtttctccaaagctgtcaccaggcaattcaggaaattattcatctggggccagtagtgcttctgcaagtggttcttcagtgaccattccacagaagatccaccagatggcagccagctatgttcaggtcacatccaacttcctctatgccaccgaaatttgggaccaagctgaacagctttccaaagagcaaaaagaattctttgctgaactggataaagtaatgggccctctcatctttaatgcaagcatcatgacagatctagttcgttatacccggcagggactgcactggcttcgccaggatgccaagttgatatcttga

SEQ ID NO: 9 - DNA, 1119 bp, Homo sapiens
  source 1..1119: mol_type = genomic DNA
atggcgaagcagtacgactcggtggagtgccctttttgtgatgaagtttccaaatacgagaagctcgccaagatcggccaaggcaccttcggggaggtgttcaaggccaggcaccgcaagaccggccagaaggtggctctgaagaaggtgctgatggaaaacgagaaggaggggttccccattacagccttgcgggagatcaagatccttcagcttctaaaacacgagaatgtggtcaacttgattgagatttgtcgaaccaaagcttccccctataaccgctgcaagggtagtatatacctggtgttcgacttctgcgagcatgaccttgctgggctgttgagcaatgttttggtcaagttcacgctgtctgagatcaagagggtgatgcagatgctgcttaacggcctctactacatccacagaaacaagatcctgcatagggacatgaaggctgctaatgtgcttatcactcgtgatggggtcctgaagctggcagactttgggctggcccgggccttcagcctggccaagaacagccagcccaaccgctacaccaaccgtgtggtgacactctggtaccggcccccggagctgttgctcggggagcgggactacggcccccccattgacctgtggggtgctgggtgcatcatggcagagatgtggacccgcagccccatcatgcagggcaacacggagcagcaccaactcgccctcatcagtcagctctgcggctccatcacccctgaggtgtggccaaacgtggacaactatgagctgtacgaaaagctggagctggtcaagggccagaagcggaaggtgaaggacaggctgaaggcctatgtgcgtgacccatacgcactggacctcatcgacaagctgctggtgctggaccctgcccagcgcatcgacagcgatgacgccctcaaccacgacttcttctggtccgaccccatgccctccgacctcaagggcatgctctccacccacctgacgtccatgttcgagtacttggcaccaccgcgccggaagggcagccagatcacccagcagtccaccaaccagagtcgcaatcccgccaccaccaaccagacggagtttgagcgcgtcttctga

SEQ ID NO: 10 - DNA, 2181 bp, Homo sapiens
  source 1..2181: mol_type = genomic DNA
atggagggagagaggaagaacaacaacaaacggtggtatttcactcgagaacagctggaaaatagcccatcccgtcgttttggcgtggacccagataaagaactttcttatcgccagcaggcggccaatctgcttcaggacatggggcagcgtcttaacgtctcacaattgactatcaacactgctatagtatacatgcatcgattctacatgattcagtccttcacacggttccctggaaattctgtggctccagcagccttgtttctagcagctaaagtggaggagcagcccaaaaaattggaacatgtcatcaaggtagcacatacttgtctccatcctcaggaatcccttcctgatactagaagtgaggcttatttgcaacaagttcaagatctggtcattttagaaagcataattttgcagactttaggctttgaactaacaattgatcacccacatactcatgtagtaaagtgcactcaacttgttcgagcaagcaaggacttagcacagacttcttacttcatggcaaccaacagcctgcatttgaccacatttagcctgcagtacacacctcctgtggtggcctgtgtctgcattcacctggcttgcaagtggtccaattgggagatcccagtctcaactgacgggaagcactggtgggagtatgttgacgccactgtgaccttggaacttttagatgaactgacacatgagtttctacagattttggagaaaactcccaacaggctcaaacgcatttggaattggagggcatgcgaggctgccaagaaaacaaaagcagatgaccgaggaacagatgaaaagacttcagagcagacaatcctcaatatgatttcccagagctcttcagacacaaccattgcaggtttaatgagcatgtcaacttctaccacaagtgcagtgccttccctgccagtctccgaagagtcatccagcaacttaaccagtgtggagatgttgccgggcaagcgttggctgtcctcccaaccttctttcaaactagaacctactcagggtcatcggactagtgagaatttagcacttacaggagttgatcattccttaccacaggatggttcaaatgcatttatttcccagaagcagaatagtaagagtgtgccatcagctaaagtgtcactgaaagaataccgcgcgaagcatgcagaagaattggctgcccagaagaggcaactggagaacatggaagccaatgtgaagtcacaatatgcatatgctgcccagaatctcctttctcatcatgatagccattcttcagtcattctaaaaatgcccatagagggttcagaaaaccccgagcggccttttctggaaaaggctgacaaaacagctctcaaaatgagaatcccagtggcaggtggagataaagctgcgtcttcaaaaccagaggagataaaaatgcgcataaaagtccatgctgcagctgataagcacaattctgtagaggacagtgttacaaagagccgagagcacaaagaaaagcacaagactcacccatctaatcatcatcatcatcataatcaccactcacacaagcactctcattcccaacttccagttggtactgggaacaaacgtcctggtgatccaaaacatagtagccagacaagcaacttagcacataaaacctatagcttgtctagttctttttcctcttccagttctactcgtaaaaggggaccctctgaagagactggaggggctgtgtttgatcatccagccaagattgccaagagtactaaatcctcttccctaaatttctccttcccttcacttcctacaatgggtcagatgcctgggcatagctcagacacaagtggcctttccttttcacagcccagctgtaaaactcgtgtccctcattcgaaactggataaagggcccactggggccaatggtcacaacacgacccagacaatagactatcaagacactgtgaatatgcttcactccctgctcagtgcccagggtgttcagcccactcagcccactgcatttgaatttgttcgtccttatagtgactatctgaatcctcggtctggtggaatctcctcgagatctggcaatacagacaaaccccggccaccacctctgccatcagaacctcctccaccacttccaccccttcctaagtaa

SEQ ID NO: 11 - DNA, 1992 bp, Homo sapiens
  source 1..1992: mol_type = genomic DNA
atggcgtcgggccgtggagcttcttctcgctggttctttactcgggaacagctggagaacacgccgagccgccgctgcggagtggaggcggataaagagctctcgtgccgccagcaggcggccaacctcatccaggagatgggacagcgtctcaatgtctctcagcttacaataaacactgcgattgtttatatgcacaggttttatatgcaccattctttcaccaaattcaacaaaaatataatatcgtctactgcattatttttggctgcaaaagtggaagaacaggctcgaaaacttgaacatgttatcaaagtagcacatgcttgtcttcatcctctagagccactgctggatactaaatgtgatgcttaccttcaacagactcaagaactggttatacttgaaaccataatgctacaaactctaggttttgagatcaccattgaacacccacacacagatgtggtgaaatgtacccagttagtaagagcaagcaaggatttggcacagacatcctatttcatggctaccaacagtctgcatcttacaaccttctgtcttcagtacaaaccaacagtgatagcatgtgtatgcattcatttggcttgcaaatggtccaattgggagatccctgtatcaactgatggaaagcattggtgggaatatgtggatcctacagttactctagaattattagatgagctaacacatgagtttctacaaatattggagaaaacgcctaataggttgaagaagattcgaaactggagggctaatcaggcagctaggaaaccaaaagtagatggacaggtatcagagacaccacttcttggttcatctttggtccagaattccattttagtagatagtgtcactggtgtgcctacaaacccaagttttcagaaaccatctacatcagcattccctgcgccagtacctctaaattcaggaaatatttctgttcaagacagccatacatctgataatttgtcaatgctagcaacaggaatgccaagtacttcatacggtttatcatcacaccaggaatggcctcaacatcaagactcagcaaggacagaacagctatattcacagaaacaggagacatctttgtctggtagccagtacaacatcaacttccagcagggaccttctatatcactgcattcaggattacatcacagacctgacaaaatttcagatcattcttctgttaagcaagaatatactcataaagcagggagcagtaaacaccatgggccaatttccactactccaggaataattcctcagaaaatgtctttagataaatatagagaaaagcgtaaactagaaactcttgatctcgatgtaagggatcattatatagctgcccaggtagaacagcagcacaaacaagggcagtcacaggcagccagcagcagttctgttacttctcccattaaaatgaaaatacctatcgcaaatactgaaaaatacatggcagataaaaaggaaaagagtgggtcactgaaattacggattccaataccacccactgataaaagcgccagtaaagaagaactgaaaatgaaaataaaagtttcttcttcagaaagacacagctcttctgatgaaggcagtgggaaaagcaaacattcaagcccacatattagcagagaccataaggagaagcacaaggagcatccttcaagccgccaccacaccagcagccacaagcattcccactcgcatagtggcagcagcagcggtggcagtaaacacagtgccgacggaataccacccactgttctgaggagtcctgttggcctgagcagtgatggcatttcctctagctccagctcttcaaggaagaggctgcatgtcaatgatgcatctcacaaccaccactccaaaatgagcaaaagttccaaaagttcaggtgggctacggacatctcagcaccctcgtgaaactggacaagaagccagtggagaccaacggtcctga

SEQ ID NO: 12 - protein, 372 aa, Homo sapiens
MAKQYDSVECPFCDEVSKYEKLAKIGQGTFGEVFKARHRKTGQKVALKKVLMENEKEGFPITALREIKILQLLKHENVVNLIEICRTKASPYNRCKGSIYLVFDFCEHDLAGLLSNVLVKFTLSEIKRVMQMLLNGLYYIHRNKILHRDMKAANVLITRDGVLKLADFGLARAFSLAKNSQPNRYTNRVVTLWYRPPELLLGERDYGPPIDLWGAGCIMAEMWTRSPIMQGNTEQHQLALISQLCGSITPEVWPNVDNYELYEKLELVKGQKRKVKDRLKAYVRDPYALDLIDKLLVLDPAQRIDSDDALNHDFFWSDPMPSDLKGMLSTHLTSMFEYLAPPRRKGSQITQQSTNQSRNPATTNQTEFERVF

SEQ ID NO: 13 - protein, 568 aa, Homo sapiens
MASSCAVQVKLELGHRAQVRKKPTVEGFTHDWMVFVRGPEHSNIQHFVEKVVFHLHESFPRPKRVCKDPPYKVEESGYAGFILPIEVYFKNKEEPRKVRFDYDLFLHLEGHPPVNHLRCEKLTFNNPTEDFRRKLLKAGGDPNRSIHTSSSSSSSSSSSSSSSSSSSSSSSSSSSSSSSSSSSSSSSSSSTSFSKPHKLMKEHKEKPSKDSREHKSAFKEPSRDHNKSSKESSKKPKENKPLKEEKIVPKMAFKEPKPMSKEPKPDSNLLTITSGQDKKAPSKRPPISDSEELSAKKRKKSSSEALFKSFSSAPPLILTCSADKKQIKDKSHVKMGKVKIESETSEKKKSTLPPFDDIVDPNDSDVEENISSKSDSEQPSPASSSSSSSSSFTPSQTRQQGPLRSIMKDLHSDDNEEESDEVEDNDNDSEMERPVNRGGSRSRRVSLSDGSDSESSSASSPLHHEPPPPLLKTNNNQILEVKSPIKQSKSDKQIKNGECDKAYLDELVELHRRLMTLRERHILQQIVNLIEETGHFHITNTTFDFDLCSLDKTTVRKLQSYLETSGTS

SEQ ID NO: 14 - DNA, 25 bp, synthetic construct
  source 1..25: mol_type = other DNA
  modified_base 25: mod_base = OTHER; note = 3'inverted deoxythymidine
tccgacgatcccacgttcccgtggt

SEQ ID NO: 15 - DNA, 66 bp, synthetic construct
  source 1..66: mol_type = other DNA
ccaacaattttacaaactcttctgtttttccttgctttatttattccatctattaaggaatgaaac

SEQ ID NO: 16 - DNA, 66 bp, synthetic construct
  source 1..66: mol_type = other DNA
ccaacaattttacaaactcttctgtttttccttgcttgtagtattccagctataaggtaatgaaac

SEQ ID NO: 17 - DNA, 66 bp, synthetic construct
  source 1..66: mol_type = other DNA
gtccaaatttacctgctgcttccgccaattttacaaattcttctgtttttccttttttgcattatt

SEQ ID NO: 18 - DNA, 66 bp, synthetic construct
  source 1..66: mol_type = other DNA
gtccaaattgccatctatacgtgctgcttccaacaattttacaaactcttctgtttttccttgctt

SEQ ID NO: 19 - DNA, 66 bp, synthetic construct
  source 1..66: mol_type = other DNA
ctttttcatggtctctatagtccaaattgccatctatacgtgctgcttccaacaattttacaaact

SEQ ID NO: 20 - DNA, 66 bp, synthetic construct; identical to SEQ ID NO: 19
  source 1..66: mol_type = other DNA

SEQ ID NO: 21 - DNA, 66 bp, synthetic construct
  source 1..66: mol_type = other DNA
catctggtctttttcatggtctctatagtccaaattgccatctatacctgctgcttccaacaattt

SEQ ID NO: 22 - DNA, 66 bp, synthetic construct
  source 1..66: mol_type = other DNA
catctggtctttttcatggtctctatagtccaaattgccatctatacgtgctgcttccaacaattt

(19) United States
(12) Patent Application Publication
(10) Pub. No.: US 2024/0390382 A1
(43) Pub. Date: Nov. 28, 2024

(54) METHOD FOR TREATING CANCER

(71) Applicant: THE CHILDREN'S MEDICAL CENTER CORPORATION, Boston, MA (US)

(72) Inventors: Vijay G. Sankaran, Boston, MA (US); Jiawei Zhao, Boston, MA (US); Liam D. Cato, Boston, MA (US)

(73) Assignee: THE CHILDREN'S MEDICAL CENTER CORPORATION, Boston, MA (US)

(21) Appl. No.: 18/695,264

(22) PCT Filed: Sep. 22, 2022
(86) PCT No.: PCT/US2022/044354

§ 371 Date: Mar. 25, 2024

Related U.S. Application Data
(60) Provisional application No. 63/249,382, filed on Sep. 28, 2021.
(60) Provisional application No. 63/280,828, filed on Nov. 18, 2021.

(51) Int. Cl. A61K 31/53; A61K 31/164; A61K 31/352; A61K 31/426; A61K 31/437; A61K 31/4439; A61K 31/444; A61K 31/506; A61K 31/52; A61K 45/06

(52) U.S. Cl. CPC A61K 31/53; A61K 31/164; A61K 31/352; A61K 31/426; A61K 31/437; A61K 31/4439; A61K 31/444; A61K 31/506; A61K 31/52; A61K 45/06

(57) ABSTRACT

Described herein are methods and compositions for treating cancer. Aspects of the invention relate to administering to a subject having cancer an inhibitor of the super elongation complex (SEC). In various embodiments, the cancer is a blood cancer. Wherein the blood cancer is selected from the group consisting of leukemia, lymphoma, myeloma, and myeloproliferative neoplasms (MPNs).

### CROSS-REFERENCE TO RELATED APPLICATIONS

This application is a 35 U.S.C. § 371 National Phase Entry Application of International Application No. PCT/US2022/044354 filed Sep. 22, 2022, which designates the U.S., and which claims benefit under 35 U.S.C. § 119(e) of U.S. Provisional Application No. 63/249,382 filed on Sep. 28, 2021 and U.S. Provisional Application No. 63/280,828 filed on Nov. 18, 2021, the contents of each of which are incorporated herein by reference in their entireties.

### GOVERNMENT SUPPORT

This invention was made with Government support under Grant Nos DK103794 and HL146500 awarded by the National Institutes of Health. The Government has certain rights in the invention.

### FIELD OF THE INVENTION

The field of the invention relates to the treatment of cancer.

### SEQUENCE LISTING

The instant application contains a Sequence Listin which has been submitted electronically in XML format and is hereby incorporated by reference in its entirety. Said XML copy, created on Oct. 22, 2022, is named 701039-190710USPX_SL.xml and is 45,056 bytes in size.

### BACKGROUND

Blood cancers, also called hematologic cancers, occur when the bone marrow produces abnormal or excessive amounts of blood cells. The main subtypes of blood cancer are leukemias, lymphomas, myeloma, and myeloproliferative neoplasms (MPN). Leukemia occurs when the body creates too many abnormal white blood cells, lymphomas develop in the lymphatic system. myelomas originate in the blood's plasma cells, and MPN are slowly developing cancers that stem from the blood stem cells excessively becoming one or more mature blood cell type. Despite vast research, however, a significant heritable component for these diseases remains poorly understood, thus preventing efficient treatments. The overarching premise of this research is to identify additional inherited risk factors for blood cancers and to understand the aberrant pathways that ultimately lead to disease. By uncovering new risk factors and pathways, more effective and efficient agents can be utilized to target, treat, or prevent these diseases. Data presented herein show that variants in the CTR9 gene, which codes for a subunit of the PAF1 transcription elongation complex increase risk of MPNs tenfold; and that this predisposition stems from the variants causing increased expression and activity of the super elongation complex (SEC). These data further reveal that inhibition of the SEC can rescue excess hematopoietic stem cell self-renewal, in both a CTR9-mediated and CTR9 non-mediated manner, revealing a novel target for treating blood cancers.

### SUMMARY OF THE INVENTION

One aspect described herein provides a method for treating cancer comprising administering to a subject having cancer an agent that inhibits the super elongation complex (SEC).

In one embodiment of any aspect described herein, the cancer is a blood cancer. Exemplary blood cancers include leukemia, lymphoma, myeloma, and myeloproliferative neoplasm.

In one embodiment of any aspect described herein, the leukemia is selected from a group consisting of Acute myeloid leukemia (AML), Chronic myeloid leukemia (CML), Acute lymphocytic leukemia (ALL), and Chronic lymphocytic leukemia (CLL).

In one embodiment of any aspect described herein, the lymphoma is selected from the group consisting of a non-Hodgkin lymphoma, Hodgkin lymphoma, Diffuse large B-cell lymphoma (DLBCL), Follicular lymphoma, Chronic lymphocytic leukemia (CLL), Small lymphocytic lymphoma (SLL), Mantle cell lymphoma (MCL), Marginal zone lymphomas, and Burkitt lymphoma.

In one embodiment of any aspect described herein, the myeloma is selected from the group consisting of multiple myeloma, plasmacytoma, and monoclonal gammopathy of undetermined significance (MGUS).

In one embodiment of any aspect described herein, the MPN is selected from the group consisting of Polycythemia vera (PV), Essential thrombocythemia (ET), and Myelofibrosis (MF).

In one embodiment of any aspect described herein, the cancer results from or comprises a mutation that impacts hematopoietic stem cell self-renewal. Exemplary mutations include mutations in the CTR9 gene or other components of the PAF1 complex.

In one embodiment of any aspect described herein, the agent inhibits at least one component of the SEC, which is comprised of subunits including the proteins Eleven-nineteen lysine-rich leukemia (ELL) 1, ELL2, ELL3, ELL-associated factor (EAF) 1/, EAF2, Mixed-lineage leukemia translocated to chromosome 3 (MLLT3) protein AF-9 (AF9), Mixed-lineage leukemia translocated to chromosome 1 (MLLT1) protein ENL, AF4/FMR2 family member (AFF) 1, AFF4, and positive transcription elongation factor (P-TEFb).

In one embodiment of any aspect described herein, the P-TEFb subcomplex consists of cyclin-dependent kinase 9 (CDK9), cyclin T (CycT) 1, CycT2, CycT3, bromodomain containing 4 protein (BRD4), and 7SK snRNP.

In one embodiment of any aspect described herein, the agent that inhibits the SEC is selected from the group consisting of a small molecule inhibitor, a small molecule degrader (proteolysis-targeting chimera, PROTAC), an antibody, a peptide, a genome editing system, an antisense oligonucleotide, and an RNAi.

In one embodiment of any aspect described herein, the small molecule is selected from the group consisting of: SR-0813, LDCO000067, AZD4573, atuveciclib, flavopiridol, CR8, indirubin-3′-monoxime derivatives, 5-fluoro-N²,N⁴-diphenylpyrimidine-2,4-diamines, 4-(thiazol-5-ul)-2-(phenylamino)pyrimidines, TG02, CDKI-73, 2,4,5-trisubstited pyrimidine derivatives, Wogonin, PHA-767491, LY2857785, dinaciclib, roscovitine, voruciclib, sns-032, P276-00, FIT-039, CCT068127, MC180295, and KL-1, SR-1114, dTAG13, A-1592668.

In one embodiment of any aspect described herein, the RNAi is a microRNA, an siRNA, or a shRNA.

In one embodiment of any aspect described herein, the agent that inhibits the SEC is administered at least once.

In one embodiment of any aspect described herein, the subject has previously been administered an anti-cancer therapy. In one embodiment of any aspect described herein, the subject has not previously been administered an anti-cancer therapy.

In one embodiment of any aspect described herein, the method further comprises the step of, after the step of administering, administering at least one anti-cancer therapy to the subject. In one embodiment of any aspect described herein, the method further comprises the step of, prior the step of administering, administering at least one anti-cancer therapy to the subject. Exemplary anti-cancer therapies include low dose chemotherapy.

In one embodiment of any aspect described herein, the method further comprises the step of, prior to administering, diagnosing a subject as having cancer.

In one embodiment of any aspect described herein, the method further comprises the step of, prior to administering, receiving a result from an assay that diagnoses a subject as having cancer.

In one embodiment of any aspect described herein, the anti-cancer treatment is selected from the group consisting of: chemotherapy, hematopoietic stem cell transplant, radiation, chemo-radiation, surgery, chimeric antigen receptor T-cells, immune checkpoint inhibitor, an antibody targeting an antigen on cancer cells, a toxin-conjugated antibody targeting an antigen on cancer cells, and a bispecific antibody that recruits a normal immune cell to a tumor cell by simultaneously binding antigens expressed on each of these cells.

Another aspect described herein provides a composition comprising an agent that inhibits the SEC.

In one embodiment of any aspect described herein, the composition comprises an agent that is a small molecule inhibitor, a small molecule degrader (proteolysis-targeting chimera, PROTAC), an antibody, a peptide, a genome editing system, an antisense oligonucleotide, and an RNAi.

In one embodiment of any aspect described herein, the composition comprises a pharmaceutically acceptable carrier.

In one embodiment of any aspect described herein, the composition is used for the treatment of cancer.

### Definitions

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed technology, because the scope of the technology is limited only by the claims. Unless otherwise defined, all technical and scientific terms used herein have the same meaning as commonly understood by one of the ordinary skills in the art to which this technology belongs. If there is an apparent discrepancy between the usage of a term in the art and its definition provided herein, the definition provided within the specification shall prevail.

As used herein, the terms “treat,” “treatment,” “treating,” or “amelioration” refer to therapeutic treatments, wherein the object is to reverse, alleviate, ameliorate, inhibit, slow down, or stop the progression or severity of a condition associated with cancer, e.g., blood cancer. The term “treating” includes reducing or alleviating at least one adverse effect or symptom of cancer. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilization (i.e., not worsening) of state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

As used herein, the term “administering,” refers to the placement of a therapeutic agent (e.g., an agent that inhibits the SEC) or pharmaceutical composition thereof as disclosed herein into a subject by a method or route which results in at least partial delivery of the agent to the subject. Pharmaceutical compositions comprising agents as disclosed herein can be administered by any appropriate route which results in an effective treatment in the subject.

As used herein, a “subject” means a human or animal. Usually the animal is a vertebrate such as a primate, rodent, domestic animal or game animal. Primates include, for example, chimpanzees, cynomologous monkeys, spider monkeys, and macaques, e.g., Rhesus. Rodents include, for example, mice, rats, woodchucks, ferrets, rabbits and hamsters. Domestic and game animals include, for example, cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of disease e.g., cancer. A subject can be male or female. A subject can be an infant subject (i.e., less than one-year-old); a pediatric subject (i.e., one year to less than 18 years old); or an adult subject (i.e., more than 18 years old).

A subject can be one who has been previously diagnosed with or identified as suffering from or having a disease or disorder in need of treatment (e.g., cancer) or one or more complications related to such a disease or disorder, and optionally, have already undergone treatment (e.g., one or more cancer therapies) for the disease or disorder or the one or more complications related to the disease or disorder. Alternatively, a subject can also be one who has not been previously diagnosed as having such disease or disorder or related complications. For example, a subject can be one who exhibits one or more risk factors for the disease or disorder or one or more complications related to the disease or disorder or a subject who does not exhibit risk factors.

As used herein, an “agent” refers to e.g., a molecule, protein, peptide, antibody, or nucleic acid, that inhibits expression of a polypeptide or polynucleotide, or binds to, partially or totally blocks stimulation, decreases, prevents, delays activation, inactivates, desensitizes, or down regulates the activity of the polypeptide or the polynucleotide. Agents that inhibit the SEC can act directly or indirectly, e.g., inhibit expression through translation, post-translational processing, stability, degradation, or nuclear or cytoplasmic localization of a polypeptide, or through transcription, post transcriptional processing, stability or degradation of a polynucleotide, or bind to, partially or totally, block stimulation, DNA binding, transcription factor activity or enzymatic activity, decrease, prevent, delay activation, inactivate, desensitize, or down regulate the activity of a polypeptide or polynucleotide.

The term “agent” as used herein means any compound or substance such as, but not limited to, a small molecule, nucleic acid, polypeptide, peptide, drug, ion, etc. An “agent” can be any chemical, entity or moiety, including without limitation synthetic and naturally-occurring proteinaceous and non-proteinaceous entities. In some embodiments, an agent is nucleic acid, nucleic acid analogues, proteins, antibodies, peptides, aptamers, oligomer of nucleic acids, amino acids, or carbohydrates including without limitation proteins, oligonucleotides, ribozymes, DNAzymes, glycoproteins, siRNAs, lipoproteins, aptamers, and modifications, and combinations thereof etc. Compounds can be known to have a desired activity and/or property, or can be selected from a library of diverse compounds.

The agent can be a molecule from one or more chemical classes, e.g., organic molecules, which may include organometallic molecules, inorganic molecules, genetic sequences, etc. Agents may also be fusion proteins from one or more proteins, chimeric proteins (for example domain switching or homologous recombination of functionally significant regions of related or different molecules), synthetic proteins or other protein variations including substitutions, deletions, insertion and other variants.

As used herein, the term “small molecule” refers to a chemical agent which can include, but is not limited to, a peptide, a peptidomimetic, an amino acid, an amino acid analog, a polynucleotide, a polynucleotide analog, an aptamer, a nucleotide, a nucleotide analog, an organic or inorganic compound (e.g., including heterorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

The term “RNAi” as used herein refers to interfering RNA or RNA interference. RNAi refers to a means of selective post-transcriptional gene silencing by destruction of specific mRNA by molecules that bind and inhibit the processing of mRNA, for example inhibit mRNA translation or result in mRNA degradation. As used herein, the term “RNAi” refers to any type of interfering RNA, including but are not limited to, siRNA, shRNA, endogenous microRNA, and artificial microRNA. For instance, it includes sequences previously identified as siRNA, regardless of the mechanism of down-stream processing of the RNA (i.e. although siRNAs are believed to have a specific method of in vivo processing resulting in the cleavage of mRNA, such sequences can be incorporated into the vectors in the context of the flanking sequences described herein).

As used herein, the term “cancer therapy” or “cancer treatment” refers to a therapy useful in treating a cancer. Examples of anti-cancer therapeutic agents include, but are not limited to surgery, chemotherapeutic agents, immunotherapy, growth inhibitory agents, cytotoxic agents, agents used in radiation therapy, anti-angiogenesis agents, apoptotic agents, anti-tubulin agents, and other agents to treat cancer, such as anti-HER-2 antibodies (e.g., HERCEPTIN®), anti-CD20 antibodies, an epidermal growth factor receptor (EGFR) antagonist (e.g., a tyrosine kinase inhibitor), HER1/EGFR inhibitor (e.g., erlotinib (TARCEVA®)), platelet derived growth factor inhibitors (e.g., GLEEVEC™ (Imatinib Mesylate)), a COX-2 inhibitor (e.g., celecoxib), interferons, cytokines, antagonists (e.g., neutralizing antibodies) that bind to one or more of the following targets ErbB2, ErbB3, ErbB4, PDGFR-beta, BlyS, APRIL, BCMA or VEGF receptor(s), TRAIL/Apo2, and other bioactive and organic chemical agents, etc. Combinations thereof are also contemplated for use with the methods described herein.

As used herein, “PAF1c” refers to the PAF1 transcription elongation complex, a five-subunit eukaryotes-specific complex that helps regulate transcription elongation. Exemplary components of the PAF1c include Paf1, Ctr9, Cdc73, Leo1, and WDR61.

Methods and compositions described herein require that the levels and/or activity of the super elongation complex (SEC) are inhibited. As used herein, “SEC” refers to a multi-protein complex that helps facilitate transcription elongation checkpoint control (TECC) of developmentally regulated genes following transcriptional initiation by RNA polymerase II (Pol II). TECC is critical for regulation of gene expression during development, and its misregulation can result in the onset of diseases, e.g., cancer. Exemplary components of the SEC include the eleven-nineteen Lys-rich leukemia (ELL) family members ELL1, ELL2, and ELL3; the ELL-associated factor (EAF) family members EAF1 and EAF2; the mixed lineage leukemia (MLL) translocation partners the eleven-nineteen leukemia (ENL) or ALL1-fused gene from chromosome 9 (AF9); the AF4/FMR2 family member 1 (AFF1/AF4) or AFF4; and the Pol II elongation factor P-TEFb. Each SEC complex will only have one member of the ELL family, one member of the EAF family, one member of the MLL family, and one member of the AF4/FMR2 family, along with the P-TEFb. The SEC is further described in, e.g., Luo, Z., et al. Nature Reviews Molecular Cell Biology (2012) 13, 543-547.

The ELL components are known for a number of species, e.g., human ELL (NCBI Gene ID: 8178) polypeptide (e.g., NCBI Ref Seq NP_006523.1) and mRNA (e.g., NCBI NM_006532.4), for example, of isoform A; human ELL2 (NCBI Gene ID: 22936) polypeptide (e.g. NCBI Ref Seq NP_036213.2) and mRNA (e.g., NCBI NM_012081.6), for example, of isoform A; and human ELL3 (NCBI Gene ID: 80237) polypeptide (e.g. NCBI Ref Seq NP_079441.1) and mRNA (e.g., NCBI NM_025165.3), for example, of isoform A. ELL can refer to human versions including naturally occurring variants, molecules, and alleles thereof. ELL refer to the mammalian version of, e.g., mouse, rat, rabbit, dog, cat, cow, horse, pig, and the like.

The nucleic sequence of SEQ ID NO: 1 comprises the nucleic sequence which encodes ELL2.

| is a nucleic acid sequence that encodes ELL2. | |
|---|---|
| SEQ ID NO: 1 | |
| atggcggcgg gggggacagg gggcctgcgg gaggagcagc gctatgggct gtcgtgcgga | 60 |
| cggctggggc aggacaacat caccgtactg catgtgaagc tcaccgagac ggcgatccgg | 120 |
| gcgctcgaga cttaccagag ccacaagaat ttaattcctt ttcgaccttc aatccagttc | 180 |
| caaggactcc acgggcttgt caaaattccc aaaaatgatc ccctcaatga agttcataac | 240 |
| tttaactttt atttgtcaaa tgtgggcaaa gacaaccctc agggcagctt tgactgcatc | 300 |
| cagcaaacat tctccagctc tggagcctcc cagctcaatt gcctgggatt tatacaagat | 360 |
| aaaattacag tgtgtgcaac aaacgactcg tatcagatga cacgagaaag aatgacccag | 420 |
| gcagaggagg aatcccgcaa ccgaagcaca aaagttatca aacccggtgg accatatgta | 480 |
| gggaaaagag tgcaaattcg gaaagcacct caagctgttt cagatacagt tcctgagagg | 540 |
| aaaaggtcaa cccccatgaa ccctgcaaat acaattcgaa agacacatag cagcagcacc | 600 |
| atctctcaga ggccatacag ggacagggtg attcacttac tggccctgaa ggcctacaag | 660 |
| aaaccggagc tacttgctag actccagaaa gatggtgtca atcaaaaaga caagaactcc | 720 |
| ctgggagcaa ttctgcaaca ggtagccaat ctgaattcta aggacctctc atatacctta | 780 |
| aaggattatg tttttaaaga gcttcaaaga gactggcctg gatacagtga aatagacaga | 840 |
| cggtcattgg agtcagtgct ctctagaaaa ctaaatccgt ctcagaatgc tacaggcacc | 900 |
| agccgttcag aatctcctgt atgttctagt agagatgctg tatcttctcc tcagaaacgg | 960 |
| cttttggatt cagagtttat tgatccttta atgaataaaa aagcccgaat atctcacctg | 1020 |
| acgaacagag taccaccaac actaaatggt catttgaatc ccaccagtga aaaatcggct | 1080 |
| gcaggcctcc cactgccccc tgcggctgct gccatcccca cccctccacc gctgccttca | 1140 |
| acctatctgc ccatctcaca tcctcctcag attgtaaatt ctaactccaa ctcccctagc | 1200 |
| actccagaag gccgggggac tcaagaccta cctgttgaca gttttagtca aaacgatagt | 1260 |
| atctatgagg accagcaaga caaatatacc tctaggactt ctctggaaac cttaccccct | 1320 |
| ggttccgttc tactaaagtg tccaaagcct atggaagaaa accattcaat gtctcacaaa | 1380 |
| aagtccaaaa agaagtctaa aaaacataag gaaaaggacc aaaaaaaaa gcacgacatt | 1440 |
| gagactattg aggaaaagga ggaagatctt aagagagaag aggaaattgc caagctaaat | 1500 |
| aactccagtc caaattccag tggaggagtt aaagaggatt gcactgcctc catggaacct | 1560 |
| tcagcaattg aactcccaga ttatttgata aaatatatcg ctatcgtctc ctatgagcaa | 1620 |
| cgccagaatt ataaggatga cttcaatgca gagtatgatg agtacagagc tttgcatgcc | 1680 |
| aggatggaga ctgtagctag aagatttatc aaactagatg cacaaagaaa gcgcctttct | 1740 |
| ccaggctcaa aagagtatca gaatgttcat gaagaagtct tacaagaata tcagaagata | 1800 |
| aagcagtcta gtcccaatta ccatgaagaa aaatacagat gtgaatatct tcataacaag | 1860 |
| ctggctcaca tcaaaaggct aataggtgaa tttgaccaac agcaagcaga gtcatggtcc | 1920 |
| tag | 1923 |

The nucleic sequence of SEQ ID NO: 2 comprises the nucleic sequence which encodes ELL3.

| is a nucleic acid sequence that encodes ELL3. | |
|---|---|
| SEQ ID NO: 2 | |
| atggaggagc tccaggagcc tctgagagga cagctccggc tctgcttcac gcaagctgcc | 60 |
| cggactagcc tcttactgct caggctcaac gacgctgccc tgcgggcgct gcaagagtgt | 120 |
| cagcggcaac aggtacggcc ggtgattgct ttccaaggcc accgagggta tctgagactc | 180 |
| ccaggccctg gttggtcctg cctcttctcc ttcatagtgt cccagtgttg tcaggagggc | 240 |
| gctggtggta gcttggacct tgtgtgccaa cgcttcctca ggtctgggcc taacagcctc | 300 |
| cactgcctgg gctcactcag ggagcgcctc attatttggg cagccatgga ttctatccca | 360 |
| gccccatcat cagttcaggg acacaacctg actgaagatg ccagacatcc tgagagttgg | 420 |
| cagaacacag gaggctattc tgaaggagat gcagtatcac agccacagat ggcactagag | 480 |
| gaggtgtcag tgtcagatcc actggcaagc aaccaaggac agtcactccc aggatcctca | 540 |
| agggagcaca tggcacagtg ggaagtgaga agccagaccc atgttccaaa cagagaacct | 600 |
| gttcaggcac tgccttcctc tgccagccgg aaacgtctgg acaagaaacg ttcagtgcct | 660 |
| gtagccactg tagaactgga agaaaagagg ttcagaactc tgcctttagt gccaagcccc | 720 |
| ctacaaggcc tgaccaatca ggatttacaa gagggagaag attgggagca agaagatgag | 780 |
| gacatggacc ccagattaga acacagttcc tcagttcaag aagattctga atccccaagt | 840 |
| cctgaagata taccagacta cctcctgcaa tacagggcca tccacagtgc agaacagcaa | 900 |
| catgcctatg agcaggactt tgagacagat tatgctgaat accgcatcct gcatgcccgt | 960 |
| gttgggactg caagccaaag gttcatagag ctgggagcag agattaaaag agttcggcga | 1020 |
| ggaactccag aatacaaggt cctggaagac aagataatcc aggaatataa aaagttcagg | 1080 |
| aagcagtacc caagttacag agaagaaaag cgtcgctgtg agtaccttca ccagaaattg | 1140 |
| tcccacatta aaggtctcat cctggagttt gaggaaaaga acaggggcag ctga | 1194 |

The EAF components are known for a number of species, e.g., human EAF1 (NCBI Gene ID: 85403) polypeptide (e.g., NCBI Ref Seq NP_149074.3) and mRNA (e.g., NCBI NM_033083.7); and human EAF2 (NCBI Gene ID: 55840) polypeptide (e.g. NCBI Ref Seq NP_001306970.1) and mRNA (e.g., NCBI NM_001320041.2), for example, of isoform 2. EAF can refer to human versions including naturally occurring variants, molecules, and alleles thereof. EAF refer to the mammalian version of, e.g., mouse, rat, rabbit, dog, cat, cow, horse, pig, and the like.

The nucleic sequence of SEQ ID NO: 3 comprises the nucleic sequence which encodes EAF1

| is a nucleic acid sequence that encodes EAF1 | |
|---|---|
| SEQ ID NO: 3 | |
| atgaatggga ccgcaaaccc gctgctggac cgcgaggaac attgcctgag gctcggggag | 60 |
| agcttcgaga agcggccgcg ggcctccttc cacactattc gttatgattt taaaccagca | 120 |
| tctatagaca cttcctgtga aggagagctt caagttggca aaggagatga agtcacaatt | 180 |
| acactgccac atatccctgg atccacacca cccatgactg tgttcaaggg gaacaaacgg | 240 |
| ccttaccaga aagactgtgt gcttattatt aatcatgaca ctggtgaata tgtgctggaa | 300 |
| aaactcagta gcagcattca ggtgaagaaa acaagagctg agggcagcag taaaatccag | 360 |
| gcccgaatgg aacagcagcc cactcgtcct ccacagacgt cacagccacc accacctcca | 420 |
| ccacctatgc cattcagagc tccaacgaag cctccagttg gacccaaaac ttctcccttg | 480 |
| aaagataacc cgtcacctga acctcagttg gatgacatca aaagagagct gagggctgaa | 540 |
| gttgacatta ttgaacaaat gagcagcagc agtgggagca gctcttcaga ctctgagagc | 600 |
| tcttcgggaa gtgatgacga tagctccagc agtggaggcg aggacaatgg cccagcctct | 660 |
| cctccgcagc cttcacacca gcagccctac aacagtaggc ctgccgttgc caatggaacc | 720 |
| agccggccac aaggaagcaa ccagctcatg aacaccctca gaaatgactt gcagttgagt | 780 |
| gagtctggca gtgacagtga tgactag | 807 |

The nucleic sequence of SEQ ID NO: 4 comprises the nucleic sequence which encodes EAF2

| is a nucleic acid sequence that encodes EAF2 | |
|---|---|
| SEQ ID NO: 4 | |
| atgaatagcg cagcgggatt ctcacaccta gaccgtcgcg agcgggttct caagttaggg | 60 |
| gagagtttcg agaagcagcc gcgctgcgcc ttccacactg tgcgctatga cttcaaacct | 120 |
| gcttctattg acacttcttc tgaaggatac cttgaggttg gtgaaggtga acaggtgacc | 180 |
| ataactctgc caaatataga aggttcaact ccaccagtaa ctgttttcaa aggttcaaaa | 240 |
| aaaccttact taaaagaatg cattttgatt attaaccatg atactggaga atgtcggcta | 300 |
| gaaaaactca gcagcaacat cactgtaaaa aaaacaagag ttgaaggaag cagtaaaatt | 360 |
| cagtatcgta aagaacaaca gcaacaacaa atgtggaatt cagccaggac tcccaatctt | 420 |
| gtaaaacatt ctccatctga agataagatg tccccagcat ctccaataga tgatatcgaa | 480 |
| agagaactga aggcagaagc tagtctaatg gaccagatga gtagttgtga tagttcatca | 540 |
| gattccaaaa gttcatcatc ttcaagtagt gaggatagtt ctagtgactc agaagatgaa | 600 |
| gattgcaaat cctctacttc tgatacaggg aattgtgtct caggacatcc taccatgaca | 660 |
| cagtacagga ttcctgatat agatgccagt cataatagat ttcgagacaa cagtggcctt | 720 |
| ctgatgaata ctttaagaaa tgatttgcag ctgagtgaat caggaagtga cagtgatgac | 780 |
| tga | 783 |

The MLL translocation partners are known for a number of species, e.g., human MLLT1 (NCBI Gene ID: 4298) polypeptide (e.g., NCBI Ref Seq NP_005925.2) and mRNA (e.g., NCBI NM_005934.4), for example, of protein ENL; and human MLLT3 (NCBI Gene ID: 4300) polypeptide (e.g. NCBI Ref Seq NP_001273620.1) and mRNA (e.g., NCBI NM_001286691.2), for example, of protein AF-9 isoform b. MLL can refer to human versions including naturally occurring variants, molecules, and alleles thereof. MLL refer to the mammalian version of, e.g., mouse, rat, rabbit, dog, cat, cow, horse, pig, and the like.

The nucleic sequence of SEQ ID NO: 5 comprises the nucleic sequence which encodes ENL

| is a nucleic acid sequence that encodes ENL | |
|---|---|
| SEQ ID NO: 5 | |
| atggacaatc agtgcaccgt ccaggtgagg ttagagctgg ggcatcgcgc ccaactgcgc | 60 |
| aagaagccca ccacggaggg gttcactcac gactggatgg tgtttgtccg cggccccgag | 120 |
| caatgtgaca tccagcactt cgtggagaag gtggtcttct ggctgcacga cagcttcccc | 180 |
| aagcccagac gcgtgtgcaa ggagcccccc tacaaagtag aggagtcggg gtacgctggc | 240 |
| ttcatcatgc ccatcgaggt gcacttcaaa aacaaggagg agccgaggaa ggtctgcttc | 300 |
| acctacgacc tgttcctgaa cctggaaggc aacccgcccg tgaaccacct gcgctgcgag | 360 |
| aagctcacct tcaacaaccc caccacggag ttccggtaca agctcctgcg ggccggcggg | 420 |
| gtgatggtaa tgcccgaagg agcagacacg gtgtccaggc ccagtcccga ctaccccatg | 480 |
| ttacccacaa ttccactctc tgccttctct gaccccaaga agaccaaacc atcccacggc | 540 |
| tccaaggacg ccaacaagga gagcagcaag acctccaagc cacacaaggt gaccaaggag | 600 |
| caccgggagc gcccccgcaa agactccgag agcaagagct cctccaagga gctggagcgt | 660 |
| gagcaggcca aaagctccaa ggacacctcg cggaagctgg gcgagggccg gctgcccaag | 720 |
| gaggagaagg cgccaccgcc caaggctgcc ttcaaggaac ccaagatggc cctgaaagag | 780 |
| accaagctgg aaagcacgtc ccccaaccct gggcccccac ccccaccccc acccccaccc | 840 |
| cgggcttcca gcaagcggcc ggccaccgcc gactcgccaa agcccagcgc caagaagcag | 900 |
| aagaagagca gctcgaaggg gtcccggagt gctccaggca cctcgccccg cacctcctcc | 960 |
| tcctcctcct tctcggacaa gaagccggcc aaggacaaga gcagcaccag aggggagaag | 1020 |
| gtgaaggccg agagtgagcc ccgggaggcc aaaaaggccc tggaggtgga ggagtccaac | 1080 |
| tcagaggacg aggcctcctt caagtccgag tctgcccagt caagcccgtc caactccagc | 1140 |
| tccagctcag actccagctc agactcagac ttcgagccat cccagaacca cagccaagga | 1200 |
| cccctgcgct ccatggtgga ggacctgcag tccgaggagt ccgacgagga cgactcttcg | 1260 |
| tcaggcgagg aggctgccgg caagaccaac ccggggaggg actccaggtt gagcttcagc | 1320 |
| gacagcgaga gtgacaacag cgccgactcc tccctgccca gccgtgagcc cccacccccc | 1380 |
| cagaagccac ccccgcccaa cagcaaggtg tcaggccgga ggagccccga gtcctgcagc | 1440 |
| aagcctgaga agatcctcaa gaagggcacc tacgacaagg cctacacgga tgagctggtg | 1500 |
| gagctacacc ggaggctgat ggcgctgcgg gagcgcaacg tgctgcagca gattgtgaat | 1560 |
| ctgatcgagg agactggcca cttcaatgtc accaacacca ccttcgactt cgacctcttc | 1620 |
| tccctggacg agaccaccgt gcgcaaactg cagagctgcc tggaggccgt ggccacatga | 1680 |

The nucleic sequence of SEQ ID NO: 6 comprises the nucleic sequence which encodes AF-9

| is a nucleic acid sequence that encodes AF-9 | |
|---|---|
| SEQ ID NO: 6 | |
| atggctagct cgtgttccgt gcaggtgaag ctggagctgg ggcaccgcgc ccaggtgagg | 60 |
| aaaaaaccca ccgtggaggg cttcacccac gactggatgg tgttcgtacg cggtccggag | 120 |
| cacagtaaca tacagcactt tgtggagaaa gtcgtcttcc acttgcacga aagctttcct | 180 |
| aggccaaaaa gagtgtgcaa agatccacct tacaaagtag aagaatctgg gtatgctggt | 240 |
| ttcattttgc caattgaagt ttattttaaa aacaaggaag aacctaggaa agtccgcttt | 300 |
| gattatgact tattcctgca tcttgaaggc catccaccag tgaatcacct ccgctgtgaa | 360 |
| aagctaactt tcaacaaccc cacagaggac tttaggagaa agttgctgaa ggcaggaggg | 420 |
| gaccctaata ggagtattca taccagcagc agcagcagca gcagcagtag cagcagcagc | 480 |
| agcagcagca gcagcagcag tagcagcagc agcagcagca gcagcagcag cagtagcagc | 540 |
| agcagtagca gcagcagcag cagcagtagt accagttttt caaagcctca caaattaatg | 600 |
| aaggagcaca aggaaaaacc ttctaaagac tccagagaac ataaaagtgc cttcaaagaa | 660 |
| ccttccaggg atcacaacaa atcttccaaa gaatcctcta agaaacccaa agaaaataaa | 720 |
| ccactgaaag aagagaaaat agttcctaag atggccttca aggaacctaa acccatgtca | 780 |
| aaagagccaa aaccagatag taacttactc accatcacca gtggacaaga taagaaggct | 840 |
| cctagtaaaa ggccgcccat ttcagattct gaagaactct cagccaaaaa aaggaaaaag | 900 |
| agtagctcag aggctttatt taaaagtttt tctagcgcac caccactgat actcacttgt | 960 |
| tctgctgaca aaaaacagat aaaagataaa tctcatgtca agatgggaaa ggtcaaaatt | 1020 |
| gaaagtgaga catcagagaa gaagaaatca acgttaccgc catttgatga tattgtggat | 1080 |
| cccaatgatt cagatgtgga ggagaatata tcctctaaat ctgattctga acaacccagt | 1140 |
| cctgccagct ccagctccag ctccagctcc agcttcacac catcccagac caggcaacaa | 1200 |
| ggtcctttga ggtctataat gaaagatctg cattctgatg acaatgagga ggaatcagat | 1260 |
| gaagtggagg ataacgacaa tgactctgaa atggagaggc ctgtaaatag aggaggcagc | 1320 |
| cgaagtcgca gagttagctt aagtgatggc agcgatagtg aaagcagttc tgcttcttca | 1380 |
| cccctacatc acgaacctcc accaccctta ctaaaaacca acaacaacca gattcttgaa | 1440 |
| gtgaaaagtc caataaagca aagcaaatca gataagcaaa taaagaatgg tgaatgtgac | 1500 |
| aaggcatacc tagatgaact ggtagagctt cacagaaggt taatgacatt gagagaaaga | 1560 |
| cacattctgc agcagatcgt gaaccttata gaagaaactg gacactttca tatcacaaac | 1620 |
| acaacatttg attttgatct ttgctcgctg gacaaaacca cagtccgtaa actacagagt | 1680 |
| tacctggaaa catctggaac atcctga | 1707 |

The AF4/FMR2 components are known for a number of species, e.g., human AFF1 (NCBI Gene ID: 4299) polypeptide (e.g., NCBI Ref Seq NP_001160165.1) and mRNA (e.g., NCBI NM_001166693.3), for example, of isoform 1; and human AFF4 (NCBI Gene ID: 27125) polypeptide (e.g. NCBI Ref Seq NP_055238.1) and mRNA (e.g., NCBI NM_014423.4), for example, of isoform A. EAF can refer to human versions including naturally occurring variants, molecules, and alleles thereof. EAF refer to the mammalian version of, e.g., mouse, rat, rabbit, dog, cat, cow, horse, pig, and the like.

The nucleic sequence of SEQ ID NO: 7 comprises the nucleic sequence which encodes AFFI.

| is a nucleic acid sequence that encodes AFF1. | |
|---|---|
| SEQ ID NO: 7 | |
| atggcagccc agtcaagttt gtacaatgac gacagaaacc tgcttcgaat tagagagaag | 60 |
| gaaagacgca accaggaagc ccaccaagag aaagaggcat ttcctgaaaa gattcccctt | 120 |
| tttggagagc cctacaagac agcaaaaggt gatgagctgt ctagtcgaat acagaacatg | 180 |
| ttgggaaact acgaagaagt gaaggagttc cttagtacta agtctcacac tcatcgcctg | 240 |
| gatgcttctg aaaataggtt gggaaagccg aaatatcctt taattcctga caaagggagc | 300 |
| agcattccat ccagctcctt ccacactagt gtccaccacc agtccattca cactcctgcg | 360 |
| tctggaccac tttctgttgg caacattagc cacaatccaa agatggcgca gccaagaact | 420 |
| gaaccaatgc caagtctcca tgccaaaagc tgcggcccac cggacagcca gcacctgacc | 480 |
| caggatcgcc ttggtcagga ggggttcggc tctagtcatc acaagaaagg tgaccgaaga | 540 |
| gctgacggag accactgtgc ttcggtgaca gattcggctc cagagaggga gctttctccc | 600 |
| ttaatctctt tgccttcccc agttccccct ttgtcaccta tacattccaa ccagcaaact | 660 |
| cttccccgga cgcaaggaag cagcaaggtt catggcagca gcaataacag taaaggctat | 720 |
| tgcccagcca aatctcccaa ggacctagca gtgaaagtcc atgataaaga gacccctcaa | 780 |
| gacagtttgg tggcccctgc ccagccgcct tctcagacat ttccacctcc ctccctcccc | 840 |
| tcaaaaagtg ttgcaatgca gcagaagccc acggcttatg tccggcccat ggatggtcaa | 900 |
| gatcaggccc ctagtgaatc ccctgaactg aaaccactgc cggaggacta tcgacagcag | 960 |
| acctttgaaa aaacagactt gaaagtgcct gccaaagcca agctcaccaa actgaagatg | 1020 |
| ccttctcagt cagttgagca gacctactcc aatgaagtcc attgtgttga agagattctg | 1080 |
| aaggaaatga cccattcatg gccgcctcct ttgacagcaa tacatacgcc tagtacagct | 1140 |
| gagccatcca agtttccttt ccctacaaag gactctcagc atgtcagttc tgtaacccaa | 1200 |
| aaccaaaaac aatatgatac atcttcaaaa actcactcaa attctcagca aggaacgtca | 1260 |
| tccatgctcg aagacgacct tcagctcagt gacagtgagg acagtgacag tgaacaaacc | 1320 |
| ccagagaagc ctccctcctc atctgcacct ccaagtgctc cacagtccct tccagaacca | 1380 |
| gtggcatcag cacattccag cagtgcagag tcagaaagca ccagtgactc agacagttcc | 1440 |
| tcagactcag agagcgagag cagttcaagt gacagcgaag aaaatgagcc cctagaaacc | 1500 |
| ccagctccgg agcctgagcc tccaacaaca aacaaatggc agctggacaa ctggctgacc | 1560 |
| aaagtcagcc agccagctgc gccaccagag ggccccagga gcacagagcc cccacggcgg | 1620 |
| cacccagaga gtaagggcag cagcgacagt gccacgagtc aggagcattc tgaatccaaa | 1680 |
| gatcctcccc ctaaaagctc cagcaaagcc ccccgggccc cacccgaagc cccccacccc | 1740 |
| ggaaagagga gctgtcagaa gtctccggca cagcaggagc ccccacaaag gcaaaccgtt | 1800 |
| ggaaccaaac aacccaaaaa acctgtcaag gcctctgccc gggcaggttc acggaccagc | 1860 |
| ctgcaggggg aaagggagcc agggcttctt ccctatggct cccgagacca gacttccaaa | 1920 |
| gacaagccca aggtgaagac gaaaggacgg ccccgggccg cagcaagcaa cgaacccaag | 1980 |
| ccagcagtgc ccccctccag tgagaagaag aagcacaaga gctccctccc tgccccctct | 2040 |
| aaggctctct caggcccaga acccgcgaag gacaatgtgg aggacaggac ccctgagcac | 2100 |
| tttgctcttg ttcccctgac tgagagccag ggcccacccc acagtggcag cggcagcagg | 2160 |
| actagtggct gccgccaagc cgtggtggtc caggaggaca gccgcaaaga cagactccca | 2220 |
| ttgcctttga gagacaccaa gctgctctca ccgctcaggg acactcctcc cccacaaagc | 2280 |
| ttgatggtga agatcaccct agacctgctc tctcggatac cccagcctcc cgggaagggg | 2340 |
| agccgccaga ggaaagcaga agataaacag ccgcccgcag ggaagaagca cagctctgag | 2400 |
| aagaggagct cagacagctc aagcaagttg gccaaaaaga gaaagggtga agcagaaaga | 2460 |
| gactgtgata acaagaaaat cagactggag aaggaaatca aatcacagtc atcttcatct | 2520 |
| tcatcctccc acaaagaatc ttctaaaaca aagccctcca ggccctcctc acagtcctca | 2580 |
| aagaaggaaa tgctcccccc gccacccgtg tcctcgtcct cccagaagcc agccaagcct | 2640 |
| gcacttaaga ggtcaaggcg ggaagcagac acctgtggcc aggaccctcc caaaagtgcc | 2700 |
| agcagtacca agagcaacca caaagactct tccattccca agcagagaag agtagagggg | 2760 |
| aagggctcca gaagctcctc ggagcacaag ggttcttccg gagatactgc aaatcctttt | 2820 |
| ccagtgcctt ctttgccaaa tggtaactct aaaccaggga agcctcaagt gaagtttgac | 2880 |
| aaacaacaag cagaccttca catgagggag gcaaaaaaga tgaagcagaa agcagagtta | 2940 |
| atgacggaca gggttggaaa ggcttttaag tacctggaag ccgtcttgtc cttcattgag | 3000 |
| tgcggaattg ccacagagtc tgaaagccag tcatccaagt cagcttactc tgtctactca | 3060 |
| gaaactgtag atctcattaa attcataatg tcattaaaat ccttctcaga tgccacagcg | 3120 |
| ccaacacaag agaaaatatt tgctgtttta tgcatgcgtt gccagtccat tttgaacatg | 3180 |
| gcgatgtttc gttgtaaaaa agacatagca ataaagtatt ctcgtactct taataaacac | 3240 |
| ttcgagagtt cttccaaagt cgcccaggca ccttctccat gcattgcaag cacaggcaca | 3300 |
| ccatcccctc tttccccaat gccttctcct gccagctccg tagggtccca gtcaagtgct | 3360 |
| ggcagtgtgg ggagcagtgg ggtggctgcc actatcagca ccccagtcac catccagaat | 3420 |
| atgacatctt cctatgtcac catcacatcc catgttctta ccgcctttga cctttgggaa | 3480 |
| caggccgagg ccctcacgag gaagaataaa gaattctttg ctcggctcag cacaaatgtg | 3540 |
| tgcaccttgg ccctcaacag cagtttggtg gacctggtgc actatacacg acagggtttt | 3600 |
| cagcagctac aagaattaac caaaacacct taa | 3633 |

The nucleic sequence of SEQ ID NO: 8 comprises the nucleic sequence which encodes AFF4

| is a nucleic acid sequence that encodes AFF4 | |
|---|---|
| SEQ ID NO: 8 | |
| atgaaccgtg aagaccggaa tgtgctgcgt atgaaagaac gggaaaggcg gaatcaggaa | 60 |
| attcagcagg gcgaagacgc cttcccacct agctctcctc tctttgcaga gccatacaaa | 120 |
| gttactagca aagaagataa gttatcaagt cgtattcaga gtatgcttgg aaactacgat | 180 |
| gaaatgaagg atttcatagg agacagatct ataccaaagc ttgttgcaat tcccaagcct | 240 |
| acagtaccac catcagcaga tgaaaaatct aacccaaatt tctttgaaca gagacatgga | 300 |
| ggctctcatc agagtagcaa atggactcca gtaggacccg cacccagcac ttctcagtct | 360 |
| cagaaacggt cctcaggctt acagagtgga catagtagcc agcggaccag cgcaggtagc | 420 |
| agtagtggca ctaacagtag tggtcagagg cacgaccgtg agtcatataa caatagtggg | 480 |
| agcagtagcc ggaaaaaagg ccagcatgga tcagaacact ccaaatcacg ttcttccagc | 540 |
| cctggaaaac cccaggctgt ttcttcatta aactctagtc attccaggtc tcatgggaat | 600 |
| gatcaccata gcaaggaaca tcaacgctcc aaatcacctc gggaccctga tgcaaactgg | 660 |
| gattctcctt cccgtgtacc tttttcaagt gggcagcact caactcaatc tttcccaccc | 720 |
| tcattgatgt caaagtccaa ttcaatgtta cagaaaccca ctgcctatgt gcggcccatg | 780 |
| gacggacagg agtccatgga accaaagctg tcctctgagc actacagcag ccaatcccat | 840 |
| ggcaacagca tgactgagct gaagcccagc agcaaagcac atctcaccaa gctgaaaata | 900 |
| ccttcccaac cactggatgc atcagcttct ggtgatgtga gctgtgtgga tgaaatccta | 960 |
| aaagagatga cgcattcatg gcctccccct ctaacggcta ttcatacacc atgcaaaaca | 1020 |
| gaaccttcca aatttccttt tccaactaag gagtctcagc agtccaattt tggcactgga | 1080 |
| gaacaaaaaa gatataatcc ttctaaaact tcaaatgggc accagtctaa atctatgtta | 1140 |
| aaagatgact taaaactaag cagcagtgaa gacagtgatg gggaacagga ttgtgataag | 1200 |
| acaatgccga ggagtacacc aggaagtaac tctgaacctt cacaccataa tagtgaagga | 1260 |
| gcagataact ccagggatga ttctagtagc cacagtggat ctgaaagcag ctctggatct | 1320 |
| gactcagaga gtgaaagtag ttccagtgac agtgaggcaa atgagccatc ccagagtgca | 1380 |
| tctcccgagc ctgaaccccc gccaacaaac aaatggcaac ttgataattg gctgaataaa | 1440 |
| gtgaacccac ataaagtgtc acccgcctct tcagtggaca gtaacatccc atcatctcaa | 1500 |
| ggctacaaaa aggaaggccg agagcagggc actgggaata gctacactga tacaagtgga | 1560 |
| cctaaagaaa cgagttccgc tactccggga cgagactcca aaaccatcca aaagggatca | 1620 |
| gaaagtgggc gtgggaggca gaaatctcct gcacagagtg acagcacaac acagagaaga | 1680 |
| actgtaggca aaaaacaacc caaaaaggct gagaaggcag ctgctgaaga gcctcgtgga | 1740 |
| ggcctgaaga tagaaagtga aacccctgta gacttggcta gcagcatgcc ctccagcaga | 1800 |
| cacaaagcag ccaccaaagg ctcaaggaaa cccaatataa agaaggagtc taagtcttcc | 1860 |
| cctcgaccta cagcagagaa aaagaaatat aagtcaacaa gtaaatcttc ccagaaatca | 1920 |
| agggaaatca tagaaacaga tacctcatcc tcagattcag atgaaagtga gagccttcct | 1980 |
| ccttcctcac aaactcctaa gtaccccgag agcaatagga ctcctgttaa accctcctca | 2040 |
| gtggaggaag aagatagctt ttttcggcaa cgaatgttct ctcctatgga agagaaggaa | 2100 |
| cttctttcac ccctcagtga gcctgatgac aggtacccac ttattgtgaa gattgacctg | 2160 |
| aatcttttga ctagaatacc aggaaagcct tacaaagaaa cagagccgcc caagggggaa | 2220 |
| aagaaaaatg tgccagaaaa gcacacgaga gaggctcaga aacaagcctc agaaaaagtt | 2280 |
| tccaacaaag gcaagaggaa gcataagaat gaagatgata accgagccag tgagagcaag | 2340 |
| aaacccaaaa cggaggacaa gaattcagca ggccataagc catccagcaa cagagagtca | 2400 |
| tctaagcaga gtgctgcaaa agaaaaggat ttgttgcctt ctcccgctgg gcctgttcct | 2460 |
| tcaaaagatc caaaaacaga gcatggctct cggaagagga ctattagtca gtcttcttcc | 2520 |
| ttaaagtcaa gcagtaacag caacaaggag acgagtggca gcagcaaaaa cagttcctcc | 2580 |
| acatcaaagc agaagaagac cgaagggaag acttccagta gctccaagga ggttaaggaa | 2640 |
| aaggctccaa gtagctcctc taactgtcct ccatctgcac caactcttga ttcttctaag | 2700 |
| cctcggagaa caaagcttgt ctttgatgac agaaattatt cagcagacca ttatttacaa | 2760 |
| gaagcaaaaa agctaaagca caatgcagat gcattgtctg ataggtttga gaaagctgta | 2820 |
| tactatcttg atgctgtggt atctttcatt gaatgtggga atgcattaga gaagaatgct | 2880 |
| caggaatcca aatccccatt ccctatgtat tcagagacgg tggatctcat caaatacact | 2940 |
| atgaagctaa agaattactt ggcaccagat gctacagctg cagataaacg actcacagta | 3000 |
| ctttgcctgc gatgcgagtc tttgctgtac ctgaggctgt tcaaactgaa gaaggaaaat | 3060 |
| gctctgaagt actcaaagac actgacagag cacctgaaga attcttataa taattctcaa | 3120 |
| gcaccatcgc ctggcttggg aagcaaagct gtggggatgc cttcccctgt ttctccaaag | 3180 |
| ctgtcaccag gcaattcagg aaattattca tctggggcca gtagtgcttc tgcaagtggt | 3240 |
| tcttcagtga ccattccaca gaagatccac cagatggcag ccagctatgt tcaggtcaca | 3300 |
| tccaacttcc tctatgccac cgaaatttgg gaccaagctg aacagctttc caaagagcaa | 3360 |
| aaagaattct ttgctgaact ggataaagta atgggccctc tcatctttaa tgcaagcatc | 3420 |
| atgacagatc tagttcgtta tacccggcag ggactgcact ggcttcgcca ggatgccaag | 3480 |
| ttgatatctt ga | 3492 |

As used herein, P-TEFb refers to the RNA polymerase II positive transcription elongation factor that comprises cyclin-dependent kinase 9 (CDK9) as a catalytic subunit and cyclin T1 (CYCT1) or CYCT2 as a regulatory subunit. P-TEFb phosphorylates the Pol II carboxy-terminal domain (CTD), the negative elongation factor (NELF), and the 5,6-dicholor-1-B-D-ribofuranosylbenzimidazole (DRB) sensitivity-inducing factor (DSIF), leading to the dissociation of NELF from Pol II and promoting Pol II to continue transcriptional elongation. The P-TEFb components are known for a number of species, e.g., human CDK9 (NCBI Gene ID: 1025) polypeptide (e.g., NCBI Ref Seq NP: 001252.1) and mRNA (e.g., NCBI NM_001261.4); human CYCT1 (NCBI Gene ID: 904) polypeptide (e.g., NCBI Ref Seq NP_001231.2) and mRNA (e.g., NCBI NM_001240.4), for example, of isoform A; and CYCT2 (NCBI Gene ID: 905) polypeptide (e.g., NCBI Ref Seq NP_001232.1) and mRNA (e.g., NCBI NM_001241.4), for example of isoform A. CDK9, CYCT1, and CYCT2 can refer to human versions including naturally occurring variants, molecules, and alleles thereof. CDK9, CYCT1, and CYCT2 refer to the mammalian version of, e.g., mouse, rat, rabbit, dog, cat, cow, horse, pig, and the like.

The nucleic sequence of SEQ ID NO: 9 comprises the nucleic sequence which encodes CDK9.

| is a nucleic acid sequence that encodes CDK9. | |
|---|---|
| SEQ ID NO: 9 | |
| atggcgaagc agtacgactc ggtggagtgc cctttttgtg atgaagtttc caaatacgag | 60 |
| aagctcgcca agatcggcca aggcaccttc ggggaggtgt tcaaggccag gcaccgcaag | 120 |
| accggccaga aggtggctct gaagaaggtg ctgatggaaa acgagaagga ggggttcccc | 180 |
| attacagcct tgcgggagat caagatcctt cagcttctaa aacacgagaa tgtggtcaac | 240 |
| ttgattgaga tttgtcgaac caaagcttcc ccctataacc gctgcaaggg tagtatatac | 300 |
| ctggtgttcg acttctgcga gcatgacctt gctgggctgt tgagcaatgt tttggtcaag | 360 |
| ttcacgctgt ctgagatcaa gagggtgatg cagatgctgc ttaacggcct ctactacatc | 420 |
| cacagaaaca agatcctgca tagggacatg aaggctgcta atgtgcttat cactcgtgat | 480 |
| ggggtcctga agctggcaga ctttgggctg gcccgggcct tcagcctggc caagaacagc | 540 |
| cagcccaacc gctacaccaa ccgtgtggtg acactctggt accggccccc ggagctgttg | 600 |
| ctcggggagc gggactacgg cccccccatt gacctgtggg gtgctgggtg catcatggca | 660 |
| gagatgtgga cccgcagccc catcatgcag ggcaacacgg agcagcacca actcgccctc | 720 |
| atcagtcagc tctgcggctc catcacccct gaggtgtggc caaacgtgga caactatgag | 780 |
| ctgtacgaaa agctggagct ggtcaagggc cagaagcgga aggtgaagga caggctgaag | 840 |
| gcctatgtgc gtgacccata cgcactggac ctcatcgaca agctgctggt gctggaccct | 900 |
| gcccagcgca tcgacagcga tgacgccctc aaccacgact tcttctggtc cgaccccatg | 960 |
| ccctccgacc tcaagggcat gctctccacc cacctgacgt ccatgttcga gtacttggca | 1020 |
| ccaccgcgcc ggaagggcag ccagatcacc cagcagtcca ccaaccagag tcgcaatccc | 1080 |
| gccaccacca accagacgga gtttgagcgc gtcttctga | 1119 |

The nucleic sequence of SEQ ID NO: 10 comprises the nucleic sequence which encodes CYCT1.

| is a nucleic acid sequence that encodes CYCT1. | |
|---|---|
| SEQ ID NO: 10 | |
| atggagggag agaggaagaa caacaacaaa cggtggtatt tcactcgaga acagctggaa | 60 |
| aatagcccat cccgtcgttt tggcgtggac ccagataaag aactttctta tcgccagcag | 120 |
| gcggccaatc tgcttcagga catggggcag cgtcttaacg tctcacaatt gactatcaac | 180 |
| actgctatag tatacatgca tcgattctac atgattcagt ccttcacacg gttccctgga | 240 |
| aattctgtgg ctccagcagc cttgtttcta gcagctaaag tggaggagca gcccaaaaaa | 300 |
| ttggaacatg tcatcaaggt agcacatact tgtctccatc ctcaggaatc ccttcctgat | 360 |
| actagaagtg aggcttattt gcaacaagtt caagatctgg tcattttaga aagcataatt | 420 |
| ttgcagactt taggctttga actaacaatt gatcacccac atactcatgt agtaaagtgc | 480 |
| actcaacttg ttcgagcaag caaggactta gcacagactt cttacttcat ggcaaccaac | 540 |
| agcctgcatt tgaccacatt tagcctgcag tacacacctc ctgtggtggc ctgtgtctgc | 600 |
| attcacctgg cttgcaagtg gtccaattgg gagatcccag tctcaactga cgggaagcac | 660 |
| tggtgggagt atgttgacgc cactgtgacc ttggaacttt tagatgaact gacacatgag | 720 |
| tttctacaga ttttggagaa aactcccaac aggctcaaac gcatttggaa ttggagggca | 780 |
| tgcgaggctg ccaagaaaac aaaagcagat gaccgaggaa cagatgaaaa gacttcagag | 840 |
| cagacaatcc tcaatatgat ttcccagagc tcttcagaca caaccattgc aggtttaatg | 900 |
| agcatgtcaa cttctaccac aagtgcagtg ccttccctgc cagtctccga agagtcatcc | 960 |
| agcaacttaa ccagtgtgga gatgttgccg ggcaagcgtt ggctgtcctc ccaaccttct | 1020 |
| ttcaaactag aacctactca gggtcatcgg actagtgaga atttagcact tacaggagtt | 1080 |
| gatcattcct taccacagga tggttcaaat gcatttattt cccagaagca gaatagtaag | 1140 |
| agtgtgccat cagctaaagt gtcactgaaa gaataccgcg cgaagcatgc agaagaattg | 1200 |
| gctgcccaga agaggcaact ggagaacatg gaagccaatg tgaagtcaca atatgcatat | 1260 |
| gctgcccaga atctcctttc tcatcatgat agccattctt cagtcattct aaaaatgccc | 1320 |
| atagagggtt cagaaaaccc cgagcggcct tttctggaaa aggctgacaa aacagctctc | 1380 |
| aaaatgagaa tcccagtggc aggtggagat aaagctgcgt cttcaaaacc agaggagata | 1440 |
| aaaatgcgca taaaagtcca tgctgcagct gataagcaca attctgtaga ggacagtgtt | 1500 |
| acaaagagcc gagagcacaa agaaaagcac aagactcacc catctaatca tcatcatcat | 1560 |
| cataatcacc actcacacaa gcactctcat tcccaacttc cagttggtac tgggaacaaa | 1620 |
| cgtcctggtg atccaaaaca tagtagccag acaagcaact tagcacataa aacctatagc | 1680 |
| ttgtctagtt ctttttcctc ttccagttct actcgtaaaa ggggaccctc tgaagagact | 1740 |
| ggaggggctg tgtttgatca tccagccaag attgccaaga gtactaaatc ctcttcccta | 1800 |
| aatttctcct tcccttcact tcctacaatg ggtcagatgc ctgggcatag ctcagacaca | 1860 |
| agtggccttt ccttttcaca gcccagctgt aaaactcgtg tccctcattc gaaactggat | 1920 |
| aaagggccca ctggggccaa tggtcacaac acgacccaga caatagacta tcaagacact | 1980 |
| gtgaatatgc ttcactccct gctcagtgcc cagggtgttc agcccactca gcccactgca | 2040 |
| tttgaatttg ttcgtcctta tagtgactat ctgaatcctc ggtctggtgg aatctcctcg | 2100 |
| agatctggca atacagacaa accccggcca ccacctctgc catcagaacc tcctccacca | 2160 |
| cttccacccc ttcctaagta a | 2181 |

The nucleic sequence of SEQ ID NO: 11 comprises the nucleic sequence which encodes CYCT2.

| is a nucleic acid sequence that encodes CYCT2. | |
|---|---|
| SEQ ID NO: 11 | |
| atggcgtcgg gccgtggagc ttcttctcgc tggttcttta ctcgggaaca gctggagaac | 60 |
| acgccgagcc gccgctgcgg agtggaggcg gataaagagc tctcgtgccg ccagcaggcg | 120 |
| gccaacctca tccaggagat gggacagcgt ctcaatgtct ctcagcttac aataaacact | 180 |
| gcgattgttt atatgcacag gttttatatg caccattctt tcaccaaatt caacaaaaat | 240 |
| ataatatcgt ctactgcatt atttttggct gcaaaagtgg aagaacaggc tcgaaaactt | 300 |
| gaacatgtta tcaaagtagc acatgcttgt cttcatcctc tagagccact gctggatact | 360 |
| aaatgtgatg cttaccttca acagactcaa gaactggtta tacttgaaac cataatgcta | 420 |
| caaactctag gttttgagat caccattgaa cacccacaca cagatgtggt gaaatgtacc | 480 |
| cagttagtaa gagcaagcaa ggatttggca cagacatcct atttcatggc taccaacagt | 540 |
| ctgcatctta caaccttctg tcttcagtac aaaccaacag tgatagcatg tgtatgcatt | 600 |
| catttggctt gcaaatggtc caattgggag atccctgtat caactgatgg aaagcattgg | 660 |
| tgggaatatg tggatcctac agttactcta gaattattag atgagctaac acatgagttt | 720 |
| ctacaaatat tggagaaaac gcctaatagg ttgaagaaga ttcgaaactg gagggctaat | 780 |
| caggcagcta ggaaaccaaa agtagatgga caggtatcag agacaccact tcttggttca | 840 |
| tctttggtcc agaattccat tttagtagat agtgtcactg gtgtgcctac aaacccaagt | 900 |
| tttcagaaac catctacatc agcattccct gcgccagtac ctctaaattc aggaaatatt | 960 |
| tctgttcaag acagccatac atctgataat ttgtcaatgc tagcaacagg aatgccaagt | 1020 |
| acttcatacg gtttatcatc acaccaggaa tggcctcaac atcaagactc agcaaggaca | 1080 |
| gaacagctat attcacagaa acaggagaca tctttgtctg gtagccagta caacatcaac | 1140 |
| ttccagcagg gaccttctat atcactgcat tcaggattac atcacagacc tgacaaaatt | 1200 |
| tcagatcatt cttctgttaa gcaagaatat actcataaag cagggagcag taaacaccat | 1260 |
| gggccaattt ccactactcc aggaataatt cctcagaaaa tgtctttaga taaatataga | 1320 |
| gaaaagcgta aactagaaac tcttgatctc gatgtaaggg atcattatat agctgcccag | 1380 |
| gtagaacagc agcacaaaca agggcagtca caggcagcca gcagcagttc tgttacttct | 1440 |
| cccattaaaa tgaaaatacc tatcgcaaat actgaaaaat acatggcaga taaaaaggaa | 1500 |
| aagagtgggt cactgaaatt acggattcca ataccaccca ctgataaaag cgccagtaaa | 1560 |
| gaagaactga aaatgaaaat aaaagtttct tcttcagaaa gacacagctc ttctgatgaa | 1620 |
| ggcagtggga aaagcaaaca ttcaagccca catattagca gagaccataa ggagaagcac | 1680 |
| aaggagcatc cttcaagccg ccaccacacc agcagccaca agcattccca ctcgcatagt | 1740 |
| ggcagcagca gcggtggcag taaacacagt gccgacggaa taccacccac tgttctgagg | 1800 |
| agtcctgttg gcctgagcag tgatggcatt tcctctagct ccagctcttc aaggaagagg | 1860 |
| ctgcatgtca atgatgcatc tcacaaccac cactccaaaa tgagcaaaag ttccaaaagt | 1920 |
| tcaggtgggc tacggacatc tcagcaccct cgtgaaactg gacaagaagc cagtggagac | 1980 |
| caacggtcct ga | 1992 |

The term “decrease”, “reduced”, “reduction”, or “inhibit” are all used herein to mean a decrease by a statistically significant amount. In some embodiments, “decrease”, “reduced”, “reduction”, or “inhibit” typically means a decrease by at least 10% as compared to an appropriate control (e.g. the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to an appropriate control.

As used herein, a “reference level” refers to a normal, otherwise unaffected cell population or tissue (e.g., a biological sample obtained from a healthy subject, or a biological sample obtained from the subject at a prior time point, e.g., a biological sample obtained from a patient prior to being diagnosed with cancer, or a biological sample that has not been contacted with an agent disclosed herein).

As used herein, an “appropriate control” refers to an untreated, otherwise identical cell or population (e.g., a subject who was not administered an agent described herein, or was administered by only a subset of agents described herein, as compared to a non-control cell).

The term “statistically significant” or “significantly” refers to statistical significance and generally means a two standard deviation (2SD) or greater difference.

As used herein the term “comprising” or “comprises” is used in reference to compositions, methods, and respective component(s) thereof, that are essential to the method or composition, yet open to the inclusion of unspecified elements, whether essential or not.

As used herein the term “consisting essentially of” refers to those elements required for a given embodiment. The term permits the presence of elements that do not materially affect the basic and novel or functional characteristic(s) of that embodiment. The term “consisting of” refers to compositions, methods, and respective components thereof as described herein, which are exclusive of any element not recited in that description of the embodiment.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

### BRIEF DESCRIPTION OF THE DRAWINGS

FIGS. 1A-1D show rare variant association from UK biobank show CTR9 among several significant genes. (FIG. 1A) Rare variant association results, collapsing of predicted loss-of-function and nonsynonymous variants that are grouped by gene. Dashed line indicates the FDR threshold of significance. (FIG. 1B) Dividing myeloid malignancies into constituent diseases, most signal for CTR9 arises from MPNs. (FIG. 1C) CTR9 shows an odds ratio of 9.6 (95% CI=4.86-19.04, SKAT-O p-value=5.47×10-7). Odds ratios for collapsing tests on genes (points 1-17, from left) are generally much larger than single variant odds on disease risk (points 18-27, from left; showing GWAS significant common variant markers for MPN disease (2)). (FIG. 1D) 25 coding exomes of CTR9 are shown with identified LoF variants, and predicted nonsynonymous variants. Variants found in myeloid malignancy cases are labelled.

FIGS. 2A-2C show CTR9 variants result in loss of PAF1 complex assembly. (FIG. 2A) Structure of CTR9-PAF1-CDC73 subcomplex with loss-of-function variants highlighted (N157S, S173F, V639I, T498S, I698V, and V701I) (PDB ID: 6TED). (FIG. 2B) Zoom in structures of CTR9 deleterious variants. Rotations relative to view in panel A. (FIG. 2C) Co-immunoprecipitation results (with quantification) of six CTR9 variants from myeloid malignancy cases in the discovery cohort.

FIGS. 3A-3F show partial loss of CTR9 results in HSC expansion. (FIG. 3A) Representative FACS plot and gating strategy of HSC in AAVS1 edited control and heterozygous edited CTR9 CD34+HSPCs three days post-nucleofection. (FIG. 3B) Quantification of CD34+CD45RA−, ST-HSC, and LT-HSC populations in AAVS1 control and CTR9 edited groups. Data are presented as mean±SD for three biological replicates. (FIG. 3C) Uniform manifold approximation and projection (UMAP) plots of 3,335 (AAVS1) and 4,154 (CTR9) CD34+CD45RA-CD90+ cells, highlighted according to average z-score normalized HSC gene signature. (FIG. 3D) Box plot of cells with z-score normalized HSC signature expression>0.2 of AAVS1 and CTR9 edit groups. (FIG. 3E) Representative light microscopy of colony forming unit assay of AAVS1 and CTR9 edit groups. Scale bar=200 m. (FIG. 3F) Quantification of total colonies of BFU-E, CFU-G, CFU-M, and CFU-GEMM in AAVS1 and CTR9 edited groups. Data are presented as mean±SD for three biological replicates.

FIGS. 4A-4G show partial loss of CTR9 increases super elongation complex activity to drive HSC self-renewal gene expression. (FIG. 4A) Volcano plot of CTR9 vs. AAVS1 gene expression in HSCs, highlighting genes that are MLLT3 regulated at the leading edge by gene set enrichment analysis (bold dots), y-axis for p-values is on a custom scale (Methods). (FIG. 4B) Gene set enrichment analysis of CTR9 vs. AAVS1 edited HSCs for the MLLT3 regulated HSC gene set. Leading edge genes are highlighted (FIG. 4C) PRO-seq metagene plots for all, MLLT3 bound, MLLT3 regulated and leading edge genes. (FIG. 4D) Quantification of pausing indices of key leading edge genes in AAVS1 control vs. CTR9 KO groups. (FIG. 4E) Quantification of CD34+CD45RA−, ST-HSC, and LT-HSC populations in CTR9 edit groups treated with DMSO vs. SI-0813. Data are presented as mean±SD for three biological replicates. (FIG. 4F) Co-immunoprecipitation of CD34+HSPCs by PAF1 and MLLT3 (AF9) with AAVS1 and CTR9 edited by two guide RNAs. (FIG. 4G) Schematic of the mechanistic model by which partial loss of CTR9 results in HSC expansion.

FIG. 5 shows a violin plot distribution of mean variant allele fraction (VAF) for the tested variants found in genes significantly associated with myeloid malignancy. Dark bars show median value. Lightly shaded area represents an approximate germline expectation of VAF=0.35-0.65.

FIG. 6 shows phenome-wide genetic association (PheWAS) of the CTR9 gene. The hashed line shows Bernoulli corrected p-value threshold (corrected for 1027 tested phenotypes). Of significance are only polycythemia vera, however other top hits are labelled and provide an interesting overlap in phenotype with known CDC73 related phenotypes.

FIG. 7 shows the relationship between CADD and InMeRF scores across the CTR9 gene. There is a clear linear relationship (line) between CADD scoring and InMeRF scoring when considering all nonsynonymous SNVs (nsSNVs) in CTR9. Intraclass correlation coefficient for CTR9 show good agreeability (=0.57).

FIG. 8 shows CHIP prevalence by age. CHIP phenotype prevalence in UKBB 200K whole exome sequenced participants is shown increasing non-linearly with age showing a similar prevalence curve to other large CHIP studies (6).

FIG. 9 shows time of myeloid malignancy diagnosis relative to sample blood draw. Grey points indicate a diagnosis of myeloid malignancy, time=0 is blood draw. Bold points indicate the cases carrying the six CTR9 deleterious mutations studied.

FIGS. 10A and 10B shows functional validation of CTR9 variants. (FIG. 10A) schematic of constructs that are used to transiently transfect in HEK 293T cells. (FIG. 10B) Co-immunoprecipitation analysis of the effect of mutant CTR9 proteins on the binding pattern of WT CTR9 proteins.

FIGS. 11A-11E show uncontrolled CTR9 editing results in LT-HSC expansion at an early point and depletion at a later time point. (FIG. 11A) Representative FACS plot of HSC phenotyping of CTR9 editing three days post-nucleofection. (FIG. 11B) Quantification of CD34⁺CD45RA⁻, ST-HSC, and LT-HSC populations three days post-nucleofection. Data are presented as mean±SD for three biological replicates. (FIG. 11C) Representative FACS plot of HSC phenotyping of CTR9 editing at seven days post-nucleofection. (FIG. 11D) Quantification of CD34⁺CD45RA⁻, ST-HSC, and LT-HSC populations seven days post-nucleofection. Data are presented as mean±SD for three biological replicates. (FIG. 11E) Western blot analysis of CTR9 expression and RNA polymerase II phosphorylation levels at different time points of culture.

FIG. 12 shows Sanger sequencing trace analysis of CTR9 edit titrations of four CTR9 sgRNAs. FIG. 12 discloses SEQ ID NOS 15-22, respectively, in order of appearance.

FIGS. 13A-13D show partial loss of CTR9 results in persistent HSC expansion. (FIG. 13A) Quantification of CTR9 editing efficiency by ICE analysis for four different guide RNAs. (FIG. 13B) Western blot analysis of expression of CTR9 and other PAF1 complex subunits, and RNA polymerase II phosphorylation at different times in culture. (FIG. 13C) Representative FACS plot of HSC phenotyping of CTR9 editing six days post-nucleofection. (FIG. 13D) Quantification of CD34+CD45RA−, ST-HSC, and LT-HSC populations six days post-nucleofection. Data are presented as mean±SD for three biological replicates.

FIGS. 14A and 14B show scRNA-seq analysis of a second independent guide targeting CTR9. (FIG. 14A) Uniform manifold approximation and projection (UMAP) plots of 3,335 (AAVS1) and 4,154 (CTR9 second guide) CD34⁺CD45RA⁻CD90⁺ cells, highlighted according to average z-score normalized HSC gene signature. (FIG. 14B) Box plot of cells with z-score normalized HSC signature expression>0.2 of AAVS1 and CTR9 edited groups by this second guide RNA.

FIGS. 15A and 15B show HOXA genes are overexpressed in CTR9 edited HSPCs. (FIG. 15A) qPCR analysis of HOXA gene expression in AAVS1 and CTR9 (untitrated) groups three days post-nucleofection. Data are presented as mean±SD for three biological replicates. (FIG. 15B) qPCR analysis of HOXA gene expression in AAVS1 and CTR9 (untitrated) groups three days and seven days post-nucleofection. Data are presented as mean±SD for three biological replicates.

FIGS. 16A-16E show partial loss of CTR9 results in enhanced MLLT3 activity. (FIG. 16A) Gene ontology (GO) analysis of upregulated and downregulated genes in the CTR9 edited group. (FIG. 16B) Representative violin plot of additional MLLT3 regulated genes (FIG. 16C) Volcano plot of the second independent guide RNA of CTR9 highlighting MLLT3 regulated genes found from leading edge gene set enrichment analyses. (FIG. 16D) Representative violin plot HSC self-renewal genes regulated by MLLT3. (FIG. 16E) Expression level of MLLT3 and MLLT1 across 18 hematopoietic lineages.

FIG. 17 shows partial loss of CTR9 results in enhanced transcription elongation of key transcription factors involved in HSC self-renewal. Representative bigWig tracks of HOXA clusters and key MLLT3 regulated leading edge genes.

FIG. 18 shows MLLT3 regulated genes showed significantly enhanced transcription elongation. Inverse volcano plot of the log 2 fold change of pausing indices of all delectable transcripts throughout the genome.

FIGS. 19A and 19B shows MLLT3 YEATS1 domain inhibitor SR-0813 rescues HSC expansion phenotype by partial loss of CTR9. (A) Representative FACS plot of HSC phenotyping of CTR9 edit group treated with DMSO control and MLLT3/MLLT1 YEATS domain inhibitor SI-0813. (B) qRT-PCR analysis of key leading edge genes in CTR9 edited cells by two independent guides treated with DMSO vs. SR-0813.

FIGS. 20A and 20B show super elongation complex component CDK9 inhibitor rescues HSC expansion resulting from partial loss of CTR9. (FIG. 20A) Representative FACS plot of CTR9 edited cells treated with DMSO or the CDK9 inhibitor. (FIG. 20B) Quantification of CD34+CD45RA−, ST-HSC, and LT-HSC populations. Data are presented as mean±SD for three biological replicates.

FIGS. 21A-21C show loss of different subunits of the PAF1 complex has distinct consequences on HSC maintenance. (FIG. 21A) Western blot validation of PAF1 expression level in titrated and untitrated PAF1 editing groups. (FIG. 21B) Quantification of CD34+CD45RA−, ST-HSC, and LT-HSC populations among PAF1 edited cells (titrated and untitrated) on day three and day six post-nucleofection. Data are presented as mean±SD for three biological replicates. (FIG. 21C) Quantification of CD34+CD45RA−, ST-HSC, and LT-HSC populations of LEO1 and CDC73 edited cells on day three and day six post-nucleofection. Data are presented as mean±SD for three biological replicates.

FIGS. 22A and 22B show loss of PAF1 and excessive loss of CTR9 result in HSC depletion. (FIG. 22A) Representative light microscopy of colony forming unit assay of untitrated CTR9, titrated PAF1 and untitrated PAF1 edited groups. Scale bar=200 m. (FIG. 22B) Quantification of total colonies of BFU-E, CFU-G, CFU-M, and CFU-GEMM in untitrated CTR9, titrated PAF1, and untitrated PAF1 edited groups. Data are presented as mean±SD for three biological replicates.

### DETAILED DESCRIPTION

### Methods of Treating Cancer Through Super Elongation Complex

The super elongation complex (SEC) is a multi-component complex that regulates the elongation stage of transcription. During transcription of developmentally regulated genes, RNA polymerase II (Pol II) first initiates transcription by binding to or being recruited to a promoter, however Pol II often enters a paused or suspended state 30-40 nucleotides downstream the transcription start site. The SEC complex promotes the elongation phase by phosphorylating the Pol II carboxy-terminal domain, as well as the negative elongation factor (NELF) and 5,6-dichloro-1β-D-ribofuranosylbenzimidazole (DRB) sensitivity-inducing factor (DSIF). As a result, NELF dissociates from Pol II and transcription continues. Transcriptional elongation is a sensitive process and misregulation is implicated in human diseases including cancers. The SEC is further described in, e.g., Luo, Z., et al. Nature Reviews Molecular Cell Biology (2012) 13, 543-547.

The invention described herein is related, in part, to the discovery that inhibition of the SEC via the administration of an agent targeting any SEC component can prevent or treat blood cancers. Specifically, treatment with an agent that inhibits any SEC component can prevent hematopoietic stem and progenitor cells (HSPCs) expansion, which confers risk for acquiring blood cancers, e.g. myeloproliferative neoplasms (MPNs).

The SEC consists of the eleven-nineteen Lys-rich leukemia (ELL) family of proteins, ELL1, ELL2, and ELL3; ELL-associated factor (EAF) 1, and EAF2; the mixed lineage leukemia (MLL) family of proteins, AF9 and ENL; AF4/FMR2 proteins AFF1 and AFF4; and the RNA polymerase II (Pol II) positive transcription factor b (P-TEFb), which itself consists of cyclin-dependent kinase 9 (CDK9) and cyclin T1 or T2 (CYCT1 and CYCT2). CDK9 catalytic subunit phosphorylates the Pol II carboxy-terminal domain, as well as the negative elongation factor (NELF) and 5,6-dichloro-1β-D-ribofuranosylbenzimidazole (DRB) sensitivity-inducing factor (DSIF). Elevated SEC expression, e.g., due to genetic variants, e.g. CTR9 mutations, causes expansion of long-term hematopoietic stem cells (HSC) and more differentiated short-term HSCs.

Thus, also provided herein is a method for treating cancer comprising administering an agent that inhibits the SEC, e.g., an agent that inhibits any component of the SEC. In one embodiment, the agent that inhibits the SEC inhibits the expression level and/or activity of any component of the SEC as compared to an appropriate control. In one embodiment, any component of the SEC is inhibited by at least 10%, at least 20%, at least 30%, at least 40%, at least 50%, at least 60%, at least 70%, at least 80%, at least 90%, at least 99% or more as compared to an appropriate control. As used herein, appropriate control refers to an otherwise identical sample that is not administered an agent that inhibits the SEC. One skilled in the art can determine if the inhibited component of the SEC or the SEC in whole has been inhibited e.g., via PCR-based assays or western-blotting to measure the mRNA and protein levels of the SEC, respectively. Functional assays that assess the SEC activity can be further used to determine if the SEC is inhibited. For example, one skilled in the art can perform gene expression analyses to determine whether genes regulated by the SEC complex are upregulated, for example, in vivo measurements of transcription elongation (e.g. PRO-seq, GRO-seq, NET-seq, TT-seq), or in vitro elongation assays with purified proteins. Alternatively, one skilled in the art can perform kinase activity analyses using western blotting or proteomics to determine whether Pol II is undergoing increased phosphorylation.

In various embodiments, methods described herein comprise the step of diagnosing a subject with having cancer (e.g., blood cancer) or receiving the results of an assay that diagnoses a subject of having cancer prior to administering the treatment (e.g., an agent that inhibits the SEC). A skilled clinician can diagnose a subject as having cancer using various assays known in the art. Exemplary assays include: (1) a physical exam, in which a skilled clinician feels areas of the subject's body for lumps that may indicate a tumor, or for abnormalities, such as changes in skin color or enlargement of an organ, that may indicate the presence of cancer; (2) laboratory tests, such as urine and blood tests, to identify abnormalities caused by cancer. For example, a common blood test can check the level, shape, and size of white blood cells, red blood cells, and platelets and may reveal an unusual number or type; (3) noninvasive imaging tests to examine your bones and internal organs for signs of cancer or tumors. Imaging tests used in diagnosing cancer may include a computerized tomography (CT) scan, bone scan, magnetic resonance imaging (MRI), positron emission tomography (PET) scan, ultrasound and X-ray, among others; and (4) biopsy, in which a skilled clinician collects a sample of cells from an area of the body that is suspected of having cancer for testing. Biopsies can be obtained using any method known in the art. Biopsy samples are examined to identify the presence of cancer cells using standards known in the art.

An assay for diagnosing cancer need not be performed by a clinician performing the methods of treating described herein. For example, a first clinician can perform the assay to diagnose and provide the results of the assay to a second clinician, who will perform the methods of treatment described herein.

In one embodiment, the subject has previously been administered an anti-cancer therapy prior to administration of the agent described herein. In an alternative embodiment, the subject has not previously been administered an anti-cancer therapy prior to administration of the agent described herein. In yet another embodiment, the subject is administered an anti-cancer therapy following administration of the agent described herein.

### Cancer

As used herein, “cancer” refers to a hyperproliferation of cells that have lost normal cellular control, resulting in unregulated growth, lack of differentiation, local tissue invasion, and metastasis. Cancers are classified based on the histological type (e.g., the tissue in which they originate) and their primary site (e.g., the location of the body the cancer first develops), and can be a carcinoma, a melanoma, a sarcoma, a myeloma, a leukemia, or a lymphoma. “Cancer” can also refer to a solid tumor. As used herein, the term “tumor” refers to an abnormal growth of cells or tissues, e.g., of malignant type or benign type. “Cancer” can be metastatic, meaning the cancer cells have disseminated from its primary site of origin and migrated to a secondary site.

In one embodiment, the cancer treated herein is a blood cancer.

Blood cancers are cancers where bone marrow, the site of blood cell production, creates abnormal or excessive amounts of red blood cells, white blood cells, or platelets.

In one embodiment of any aspect, the blood cancer treated is a leukemia, lymphoma, myeloma, or a myeloproliferative neoplasm.

Leukemia is a cancer that is often associated with the overproduction of immature white blood cells. Immature white blood cells do not function properly, rendering the patient prone to infection. Leukemia additionally affects red blood cells, and can cause poor blood clotting and fatigue due to anemia. Non-limiting subtypes of leukemia includes, acute myeloid leukemia (AML), Chronic myeloid leukemia (CML), Acute lymphocytic leukemia (ALL), or Chronic lymphocytic leukemia (CLL). Examples of leukemia include, but are not limited to, Myelogenous or granulocytic leukemia (malignancy of the myeloid and granulocytic white blood cell series), and Lymphatic, lymphocytic, or lymphoblastic leukemia (malignancy of the lymphoid and lymphocytic blood cell series),

Lymphomas develop in the glands or nodes of the lymphatic system (e.g., the spleen, tonsils, and thymus), which purifies bodily fluids and produces white blood cells, or lymphocytes. Unlike leukemia, lymphomas form solid tumors. Lymphoma can also occur in specific organs, for example the stomach, breast, or brain; this is referred to as extranodal lymphomas). Lymphomas are subclassified into two categories: Hodgkin lymphoma and Non-Hodgkin lymphoma. The presence of Reed-Sternberg cells in Hodgkin lymphoma diagnostically distinguishes Hodgkin lymphoma from Non-Hodgkin lymphoma. Non-limiting examples of lymphoma include Diffuse Large B-cell lymphoma (DLBCL), Follicular lymphoma, Chronic lymphocytic leukemia (CLL), Small lymphocytic lymphoma (SLL), Mantle cell lymphoma (MCL), Marginal zone lymphomas, Burkitt lymphoma, hairy cell leukemia (HCL). In one embodiment, the cancer is DLBCL or Follicular lymphoma.

Myelomas are cancers that originate in plasma cells of bone marrow. Non-limiting examples of myelomas include multiple myeloma, plasmacytoma and amyloidosis.

Myeloproliferative neoplasm (MPNs) are cancers in which cells in the bone marrow develop and function abnormally. Non-limiting examples of MPNs include Polycythemia vera (PV), Essential thrombocythemia (ET), Myelofibrosis (MF), chronic myelogenous leukemia, chronic neutrophilic leukemia, and chronic eosinophilic leukemia.

In one embodiment, the cancer is metastatic (e.g., the cancer has disseminated from its primary location to at least one secondary location).

In one embodiment, the cancer has relapsed following administration of a cancer therapy. A “relapsed cancer” is defined as the return of a disease or the signs and symptoms of a disease after a period of improvement.

In one embodiment, the cancer stems from mutations in the PAF1 complex. The PAF1 transcription elongation complex is a five-subunit eukaryotes-specific complex that helps regulate transcription elongation. The PAF1 complex is made up of Paf1, Ctr9, Cdc73, Leo1, and WDR61. In one embodiment, mutations reduce expression levels of PAF1 subunits by at least 10%, at least 20%, at least 30%, at least 40%, at least 50%, at least 60%, at least 70%, at least 80%, at least 90%, at least 99% or more as compared to an appropriate reference. In one embodiment, mutations reduce or alter PAF1 subunit activity by at least 10%, at least 20%, at least 30%, at least 40%, at least 50%, at least 60%, at least 70%, at least 80%, at least 90%, at least 99% or more as compared to an appropriate reference.

In one embodiment, the cancer results from a mutation that increases hematopoietic stem cell self-renewal. In one embodiment, the cancer causes a mutation that increases hematopoietic stem cell self-renewal. In one embodiment, the hematopoietic stem cell self-renewal is increased by at least 10%, at least 20%, at least 30%, at least 40%, at least 50%, at least 60%, at least 70%, at least 80%, at least 90%, at least 99% or more; or at least 1×, 2×, 3×, 4×, 5×, 6×, 7×, 8×, 9×, 10×, 15×, 20×, 25×, 30×, 35×, 40×, 45×, 50×, 100×, 500×1,000× or more as compared to an appropriate control. As used herein, an “appropriate control” refers to an otherwise identical biological sample that does not comprise a mutation that increases hematopoietic stem cell self-renewal. One skilled in the art can assess hematopoietic stem cell self-renewal using standard techniques known in the art, e.g., HSC self-renewal can be measured by (1) assessing a phenotypic marker combination of, e.g., CD34+/CD45RA−/CD90+/CD133+/EPCR+/ITGA3+, (2) using xenotransplantation assays, e.g., as described in the art, (3) using measures of molecularly defined HSCs with single cell RNA-seq, and/or using colony replating assays, e.g., as described in the art.

In one embodiment, the cancer results from a mutation in the CTR9 gene. In one embodiment, the cancer causes a mutation in the CTR9 gene. One skilled in the art can determine if a subject has a mutation in the CTR9 gene using standard techniques, e.g., genome sequencing techniques to determine if mutations in the CTR9 gene are present when compared to the wild-type CTR9 sequence.

In one embodiment, the mutation in the CTR9 gene is a loss-of-function mutation. In one embodiment, the mutation in the CTR9 gene is a deleterious mutation. In one embodiment, the mutation in the CTR9 gene is an inhibitory mutation. In one embodiment, the mutation results in a reduction or decrease of CTR9 expression level and/or activity, e.g., by at least 10%, at least 20%, at least 30%, at least 40%, at least 50%, at least 60%, at least 70%, at least 80%, at least 90%, at least 99% or more as compared to an appropriate control. As used herein, an “appropriate control” refers to an otherwise identical biological sample that does not comprise a CTR9 mutation. One skilled in the art can assess the mRNA and protein levels of CTR9, e.g., using PCR-based assays and western-blotting, respectively. Functional assays of CTR9 can performed by a skilled person to assess CTR9 activity, e.g., assessing downstream factors of CTR9.

In an alternative embodiment, the cancer treated herein is a carcinoma, a sarcoma, a melanoma, a carcinoma, or a solid tumor.

A carcinoma is a cancer that originates in an epithelial tissue. Carcinomas account for approximately 80-90% of all cancers. Carcinomas can affect organs or glands capable of secretion (e.g., breasts, lung, prostate, colon, or bladder). There are two subtypes of carcinomas: adenocarcinoma, which develops in an organ or gland, and squamous cell carcinoma, which originates in the squamous epithelium. Adenocarcinomas generally occur in mucus membranes, and are observed as a thickened plaque-like white mucosa. They often spread easily through the soft tissue where they occur. Exemplary adenocarcinomas include, but are not limited to, lung cancer, prostate cancer, pancreatic cancer, esophageal cancer, and colorectal cancer. Squamous cell carcinomas can originate from any region of the body. Examples of carcinomas include, but are not limited to, prostate cancer, colorectal cancer, microsatellite stable colon cancer, microsatellite instable colon cancer, hepatocellular carcinoma, breast cancer, lung cancer, small cell lung cancer, non-small cell lung cancer, lung adenocarcinoma, melanoma, basal cell carcinoma, squamous cell carcinoma, renal cell carcinoma, ductal carcinoma in situ, ductal carcinoma.

Sarcomas are cancers that originate in supportive and connective tissues, for example bones, tendons, cartilage, muscle, and fat. Sarcoma tumors usually resemble the tissue in which they grow. Non-limiting examples of sarcomas include, Osteosarcoma or osteogenic sarcoma (originating from bone), Chondrosarcoma (originating from cartilage), Leiomyosarcoma (originating from smooth muscle), Rhabdomyosarcoma (originating from skeletal muscle), Mesothelial sarcoma or mesothelioma (originate from membranous lining of body cavities), Fibrosarcoma (originating from fibrous tissue), Angiosarcoma or hemangioendothelioma (originating from blood vessels), Liposarcoma (originating from adipose tissue), Glioma or astrocytoma (originating from neurogenic connective tissue found in the brain), Myxosarcoma (originating from primitive embryonic connective tissue), or Mesenchymous or mixed mesodermal tumor (originating from mixed connective tissue types).

Melanoma is a type of cancer forming from pigment-containing melanocytes. Melanoma typically develops in the skin, but can occur in the mouth, intestine, or eye. In one embodiment, the cancer is a solid tumor. Non-limiting examples of solid tumors include Adrenocortical Tumor, Alveolar Soft Part Sarcoma, Chondrosarcoma, Colorectal Carcinoma, Desmoid Tumors, Desmoplastic Small Round Cell Tumor, Endocrine Tumors, Endodermal Sinus Tumor, Epithelioid Hemangioendothelioma, Ewing Sarcoma, Germ Cell Tumors (Solid Tumor), Giant Cell Tumor of Bone and Soft Tissue, Hepatoblastoma, Hepatocellular Carcinoma, Melanoma, Nephroma, Neuroblastoma, Non-Rhabdomyosarcoma Soft Tissue Sarcoma (NRSTS), Osteosarcoma, Paraspinal Sarcoma, Renal Cell Carcinoma, Retinoblastoma, Rhabdomyosarcoma, Synovial Sarcoma, and Wilms Tumor. Solid tumors can be found in bones, muscles, or organs, and can be sarcomas or carinomas.

### SEC Inhibiting Agents

In one aspect, an agent that inhibits the SEC is administered to a subject having cancer, e.g., blood cancer. In one aspect, an agent that inhibits the SEC is an agent that inhibits at least one component of the SEC. Components of the SEC that are targetable include, ELL1, ELL2, ELL3, AF4, AFF4, AF9, ENL, CDK9, CYCT1, and CYCT2. In one embodiment, the agent that inhibits the SEC is a small molecule, an antibody or antibody fragment, a small molecule degrader (proteolysis-targeting chimera, PROTAC), a peptide, an antisense oligonucleotide, a genome editing system, or an RNAi.

An agent described herein targets any component of the SEC for its inhibition. In one embodiment, an agent is considered effective for inhibiting the SEC if, for example, upon administration, it inhibits the presence, amount, activity and/or level of the SEC in the cell. In one embodiment, an agent is considered effective for inhibiting the SEC if, for example, upon administration, it inhibits the presence, amount, activity and/or level of at least one component of the SEC in the cell. In one embodiment, an agent is considered effective for inhibiting the SEC if, for example, upon administration, it inhibits the presence, amount, activity and/or level of at least one component of the SEC, and thereby inhibiting the presence, amount, activity and/or level of the SEC in the cell. In one embodiment, an agent that inhibits the level and/or activity of the SEC by at least 10%, by at least 20%, by at least 30%, by at least 40%, by at least 50%, by at least 60%, by at least 70%, by at least 80%, by at least 90%, by at least 99% or more as compared to an appropriate control. As used herein, an “appropriate control” refers to the level and/or activity of the SEC prior to administration of the agent, or the level and/or activity of the SEC in a population of cells that was not in contact with the agent. One skilled in the art can determine if the presence, amount or level of the SEC has been reduced using PCR-based assays or western blotting to assess mRNA or protein levels, respectively, of SEC components or by visualizing the SEC components via immunofluorescence of an antibody. One skilled in the art can determine if the activity of the SEC has been reduced using functional assays that assess downstream effects of the SEC, such as, determining if phosphorylation of Pol II affected.

An agent can inhibit e.g., the transcription, or the translation of the SEC components in the cell. An agent can inhibit the activity or alter the activity (e.g., such that the activity no longer occurs, or occurs at a reduced rate) of SEC components in the cell (e.g., CDK9's expression or its enzymatic activity).

The agent may function directly in the form in which it is administered. Alternatively, the agent can be modified or utilized intracellularly to produce something which inhibits the SEC, such as introduction of a nucleic acid sequence into the cell and its transcription resulting in the production of the nucleic acid and/or protein inhibitor of the SEC. In some embodiments, the agent is any chemical, entity or moiety, including without limitation synthetic and naturally-occurring non-proteinaceous entities. In certain embodiments the agent is a small molecule having a chemical moiety. For example, chemical moieties included unsubstituted or substituted alkyl, aromatic, or heterocyclyl moieties including macrolides, leptomycins and related natural products or analogues thereof. Agents can be known to have a desired activity and/or property, or can be identified from a library of diverse compounds.

In various embodiments, the agent is a small molecule that inhibits components of the SEC. Exemplary small molecular inhibitors of the SEC are known in the art and include SR-0813, LDC000067, AZD4573, atuveciclib, flavopiridol, CR8, Wogonin, PHA-767491, LY2857785, dinaciclib, roscovitine, voruciclib, sns-032, P276-00, FIT-039, CCT068127, MC180295, and KL-1. Chemical structures for exemplary small molecular inhibitors of the SEC are shown herein below in Table 1.

TABLE 1
Chemical structures for exemplary small molecular inhibitors of the SEC

| Small Molecule | |
|---|---|
| Inhibitor | |
| and known target | Chemical Structure |
| SR-0813; targets ENL | 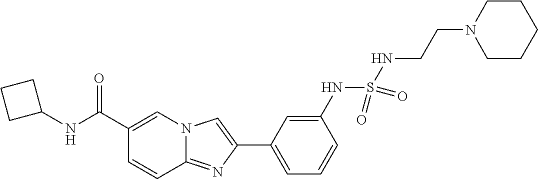 [SMILES: O=C(NC1CCC1)c1ccc2nc(-c3cccc(NS(=O)(=O)NCCN4CCCCC4)c3)cn2c1] |
| LDC000067; targets CDK9 | 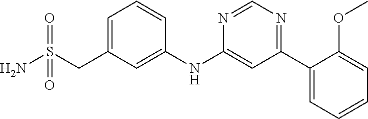 [SMILES: COc1ccccc1-c1cc(Nc2cccc(CS(N)(=O)=O)c2)ncn1] |
| AZD4573; targets CDK9 | 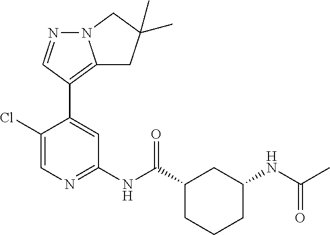 [SMILES: CC(=O)N[C@@H]1CCC[C@H](C(=O)Nc2cc(-c3cnn4c3CC(C)(C)C4)c(Cl)cn2)C1] |
| Atuveciclib (BAY 1143572); targets CDK9 | 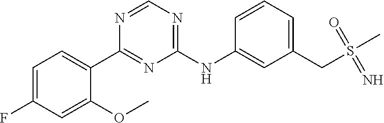 [SMILES: COc1cc(F)ccc1-c1ncnc(Nc2cccc(CS(C)(=N)=O)c2)n1] |
| Flavopiridol; targets CDK9 | 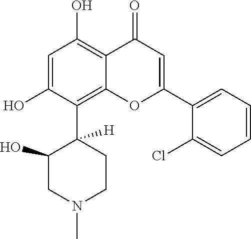 [SMILES: CN1CC[C@H](c2c(O)cc(O)c3c(=O)cc(-c4ccccc4Cl)oc23)[C@H](O)C1] |
| CR8; targets CDK9 | 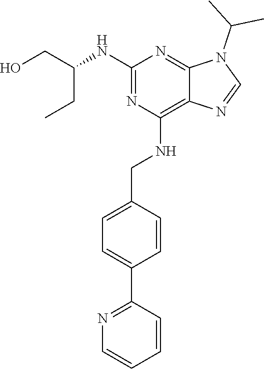 [SMILES: CC[C@H](CO)Nc1nc(NCc2ccc(-c3ccccn3)cc2)c2ncn(C(C)C)c2n1] |
| Wogonin; targets CDK9 | 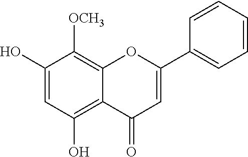 [SMILES: COc1c(O)cc(O)c2c(=O)cc(-c3ccccc3)oc12] |
| PHA-767491; targets CDK9 | 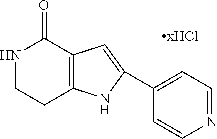 [SMILES: O=C1NCCc2[nH]c(-c3ccncc3)cc21] |
| LY2857785; targets CDK9 | 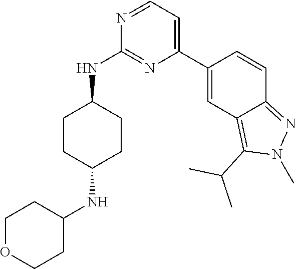 [SMILES: CC(C)c1c2cc(-c3ccnc(N[C@H]4CC[C@H](NC5CCOCC5)CC4)n3)ccc2nn1C] |
| Dinaciclib; targets CDK9 | 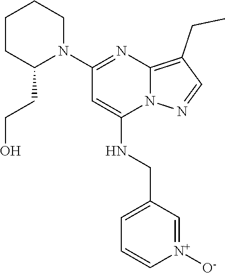 [SMILES: CCc1cnn2c(NCc3ccc[n+]([O-])c3)cc(N3CCCC[C@H]3CCO)nc12] |
| Roscovitine; targets CDK9 | 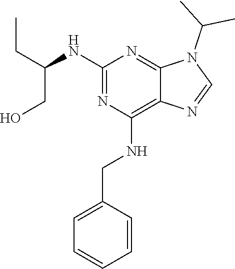 [SMILES: CC[C@H](CO)Nc1nc(NCc2ccccc2)c2ncn(C(C)C)c2n1] |
| Voruciclib; targets CDK9 | 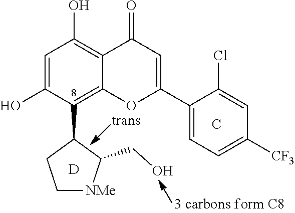 |
| sns-032; targets CDK9 | 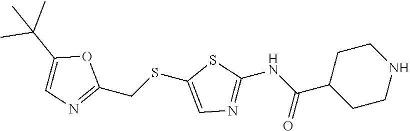 [SMILES: CC(C)(C)c1cnc(CSc2cnc(NC(=O)C3CCNCC3)s2)o1] |
| P276-00; targets CDK9 | 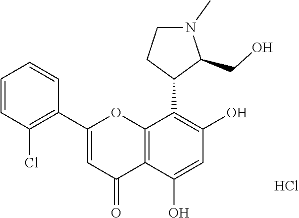 [SMILES: CN1CC[C@@H](c2c(O)cc(O)c3c(=O)cc(-c4ccccc4Cl)oc23)[C@@H]1CO ; Cl] |
| FIT-039; targets CDK9 | 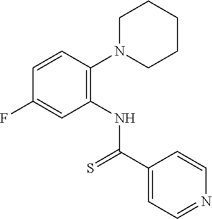 [SMILES: Fc1ccc(N2CCCCC2)c(NC(=S)c2ccncc2)c1] |
| CCT068127; targets CDK9 | 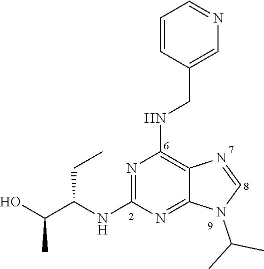 [SMILES: CC[C@H](Nc1nc(NCc2cccnc2)c2ncn(C(C)C)c2n1)[C@@H](C)O] |
| MC180295; targets CDK9 | 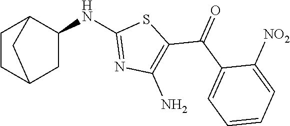 [SMILES: Nc1nc(N[C@H]2CC3CCC2C3)sc1C(=O)c1ccccc1[N+](=O)[O-]] |
| AT7519; targets CDK9 | 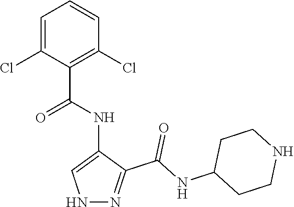 [SMILES: O=C(NC1CCNCC1)c1n[nH]cc1NC(=O)c1c(Cl)cccc1Cl] |
| CDKI-73; targets CDK9 | 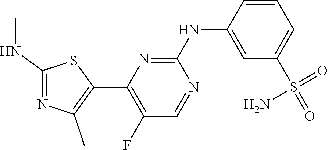 [SMILES: CNc1nc(C)c(-c2nc(Nc3cccc(S(N)(=O)=O)c3)ncc2F)s1] |
| TG02; targets CDK9 | 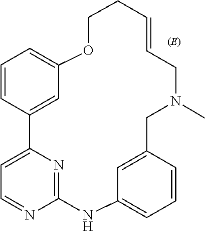 [SMILES: CN1C/C=C/CCOc2cccc(c2)-c2ccnc(n2)Nc2cccc(c2)C1] |
| KL-1; targets AFF4 | 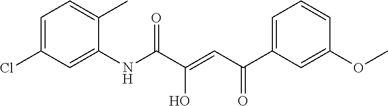 [SMILES: COc1cccc(C(=O)/C=C(\O)C(=O)Nc2cc(Cl)ccc2C)c1] |
| SR-1114; targets ENL | 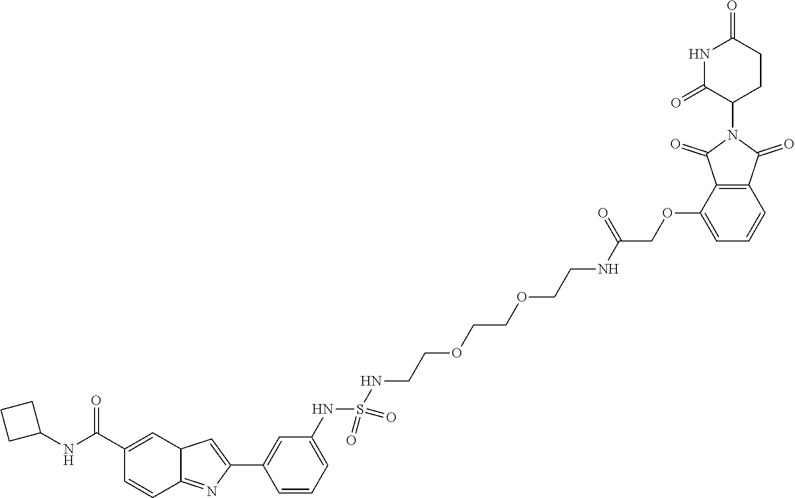 [SMILES: O=C(COc1cccc2c1C(=O)N(C1CCC(=O)NC1=O)C2=O)NCCOCCOCCNS(=O)(=O)Nc1cccc(C2=CC3C=C(C(=O)NC4CCC4)C=CC3=N2)c1] |
| dTAG-13; targets ENL/AF9 | 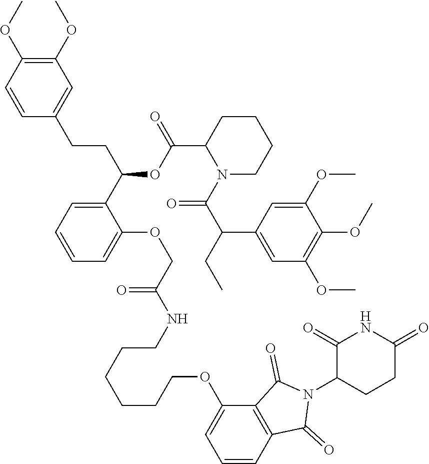 [SMILES: CCC(C(=O)N1CCCCC1C(=O)O[C@H](CCc1ccc(OC)c(OC)c1)c1ccccc1OCC(=O)NCCCCCCOc1cccc2c1C(=O)N(C1CCC(=O)NC1=O)C2=O)c1cc(OC)c(OC)c(OC)c1] |
| A-1592668; targets CDK9 | 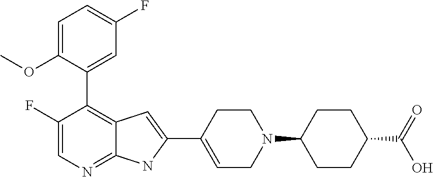 [SMILES: COc1ccc(F)cc1-c1c(F)cnc2[nH]c(C3=CCN([C@H]4CC[C@H](C(=O)O)CC4)CC3)cc12] |

Further, in one embodiment, the small molecule is a derivative, a variant, or an analog of, or is substantially similar to any of the small molecules described herein, for example as listed in Table 1. A molecule is said to be a “derivative” of another molecule when it contains additional chemical moieties not normally a part of the molecule and/or when it has been chemically modified. Such moieties can improve the molecule's expression levels, enzymatic activity, solubility, absorption, biological half-life, etc. The moieties can alternatively decrease the toxicity of the molecule, eliminate or attenuate any undesirable side effect of the molecule, etc. Moieties capable of mediating such effects are disclosed in Remington's Pharmaceutical Sciences, 18th edition, A. R. Gennaro, Ed., MackPubl., Easton, PA (1990). A “variant” of a molecule is meant to refer to a molecule substantially similar in structure and function to either the entire molecule, or to a fragment thereof. A molecule is said to be “substantially similar” to another molecule if both molecules have substantially similar structures and/or if both molecules possess a similar biological activity. Thus, provided that two molecules possess a similar activity, they are considered variants as that term is used herein even if the structure of one of the molecules not found in the other, or if the structure is not identical. An “analog” of a molecule is meant to refer to a molecule substantially similar in function to either the entire molecule or to a fragment thereof.

In one embodiment, the small molecule inhibitor of the SEC, e.g., a small molecule listed in Table 1, is conjugated to an E3 ubiquitin ligase recruitment element. As used herein, “conjugated” refers to two or more smaller entities (e.g., a small molecule and a E3 ubiquitin ligase recruitment element) that are linked, connected, associated, bonded (covalently or non-covalently), or any combination thereof, to form a larger entity. The conjugated E3 ubiquitin ligase recruitment element recruits an E3, which mediates the transfer of an ubiquitin from an E2 to the protein substrate. Binding of an ubiquitin to a protein substrate marks the protein for degradation via the ubiquitin proteasome system. Thus, a small molecule inhibitor of the SEC conjugated to an E3 ubiquitin ligase recruitment element would bind to the SEC and subsequently promote its degradation. E3 ubiquitin ligase recruitment elements can include, but are not limited to, thalidomide, lenalidomide, pomalidomide, or a VHL ligand that mimics the hydroxyproline degradation motif of HIF1-alpha. Chemical structures for exemplary E3 ubiquitin ligase recruitment element are further described in, e.g., Pavia, S L, and Crews, CM. Current Opinion in Chemical Biology. 2019. 50; 111-119, the contents of which are incorporated herein by reference in its entirety. Use of conjugated E3 ubiquitin ligase recruitment elements are further described in U.S. Pat. Nos. 7,208,157B2 and 9,770,512, the contents of which are incorporated herein by reference in its entirety.

In one embodiment, a small molecule conjugated to an E3 ubiquitin ligase recruitment element further comprises a linker. It is specifically contemplated herein that the specifications of the linker (e.g., length, sequence, etc.) would be optimized for greatest efficacy of the small molecule and E3 ubiquitin ligase recruitment element. For example, a linker would be designed such that it does not interfere with binding of the small molecule to its target (e.g., the binding pocket on the protein of interest) or the transfer of the ubiquitin from the E2 to the protein substrate.

In various embodiments, the agent that inhibits the SEC is an antibody or antigen-binding fragment thereof, or an antibody reagent that is specific for an SEC component. As used herein, the term “antibody reagent” refers to a polypeptide that includes at least one immunoglobulin variable domain or immunoglobulin variable domain sequence and which specifically binds a given antigen. An antibody reagent can comprise an antibody or a polypeptide comprising an antigen-binding domain of an antibody. In some embodiments of any of the aspects, an antibody reagent can comprise a monoclonal antibody or a polypeptide comprising an antigen-binding domain of a monoclonal antibody. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody reagent” encompasses antigen-binding fragments of antibodies (e.g., single chain antibodies, Fab and sFab fragments, F(ab′)2, Fd fragments, Fv fragments, scFv, CDRs, and domain antibody (dAb) fragments (see, e.g. de Wildt et al., Eur J. Immunol. 1996; 26(3):629-39; which is incorporated by reference herein in its entirety)) as well as complete antibodies. An antibody can have the structural features of IgA, IgG, IgE, IgD, or IgM (as well as subtypes and combinations thereof). Antibodies can be from any source, including mouse, rabbit, pig, rat, and primate (human and non-human primate) and primatized antibodies. Antibodies also include midibodies, nanobodies, humanized antibodies, chimeric antibodies, and the like.

In one embodiment, the anti-SEC antibody or antibody reagent binds to an amino acid sequence that corresponds to the SEC subunit, CDK9 based on the following amino acid sequence (SEQ ID NO: 12):

(SEQ ID NO: 12)
MAKQYDSVECPFCDEVSKYEKLAKIGQGTFGEVFKARHRKTGQKVALKK
VLMENEKEGFPITALREIKILQLLKHENVVNLIEICRTKASPYNRCKGS
IYLVFDFCEHDLAGLLSNVLVKFTLSEIKRVMQMLLNGLYYIHRNKILH
RDMKAANVLITRDGVLKLADFGLARAFSLAKNSQPNRYTNRVVTLWYRP
PELLLGERDYGPPIDLWGAGCIMAEMWTRSPIMQGNTEQHQLALISQLC
GSITPEVWPNVDNYELYEKLELVKGQKRKVKDRLKAYVRDPYALDLIDK
LLVLDPAQRIDSDDALNHDFFWSDPMPSDLKGMLSTHLTSMFEYLAPPR
RKGSQITQQSTNQSRNPATTNQTEFERVF

In another embodiment, the anti-SEC antibody or antibody reagent binds to an amino acid sequence that comprises the sequence of SEQ ID NO: 12 of CDK9, the SEC subunit; or binds to an amino acid sequence that comprises a sequence with at least 80%, at least 85%, at least 90%, at least 95%, at least 96%, at least 97%, at least 98%, at least 99% or greater sequence identity to the sequence of SEQ ID NO: 12. In one embodiment, the anti-SEC antibody or antibody reagent binds to an amino acid sequence that comprises the entire sequence of SEQ ID NO: 12 of CDK9, the SEC subunit. In another embodiment, the antibody or antibody reagent binds to an amino acid sequence that comprises a fragment of the sequence of SEQ ID NO: 12, wherein the fragment is sufficient to bind its target, e.g., CDK9, and inhibit expression and/or activity of SEC.

In one embodiment, the anti-SEC antibody or antibody reagent binds to an amino acid sequence that corresponds to the SEC subunit, AF9 based on the following amino acid sequence (SEQ ID NO: 13):

(SEQ ID NO: 13)
MASSCAVQVKLELGHRAQVRKKPTVEGFTHDWMVFVRGPEHSNIQHFVE
KVVFHLHESFPRPKRVCKDPPYKVEESGYAGFILPIEVYFKNKEEPRKV
REDYDLFLHLEGHPPVNHLRCEKLTENNPTEDFRRKLLKAGGDPNRSIH
TSSSSSSSSSSSSSSSSSSSSSSSSSSSSSSSSSSSSSSSSSSTSFSKP
HKLMKEHKEKPSKDSREHKSAFKEPSRDHNKSSKESSKKPKENKPLKEE
KIVPKMAFKEPKPMSKEPKPDSNLLTITSGQDKKAPSKRPPISDSEELS
AKKRKKSSSEALFKSFSSAPPLILTCSADKKQIKDKSHVKMGKVKIESE
TSEKKKSTLPPFDDIVDPNDSDVEENISSKSDSEQPSPASSSSSSSSSF
TPSQTRQQGPLRSIMKDLHSDDNEEESDEVEDNDNDSEMERPVNRGGSR
SRRVSLSDGSDSESSSASSPLHHEPPPPLLKINNNQILEVKSPIKQSKS
DKQIKNGECDKAYLDELVELHRRLMTLRERHILQQIVNLIEETGHFHIT
NTTFDFDLCSLDKTTVRKLQSYLETSGTS

In another embodiment, the anti-SEC antibody or antibody reagent binds to an amino acid sequence that comprises the sequence of SEQ ID NO: 13 of AF9, the SEC subunit; or binds to an amino acid sequence that comprises a sequence with at least 80%, at least 85%, at least 90%, at least 95%, at least 96%, at least 97%, at least 98%, at least 99% or greater sequence identity to the sequence of SEQ ID NO: 13. In one embodiment, the anti-SEC antibody or antibody reagent binds to an amino acid sequence that comprises the entire sequence of SEQ ID NO: 13 of AF9, the SEC subunit. In another embodiment, the antibody or antibody reagent binds to an amino acid sequence that comprises a fragment of the sequence of SEQ ID NO: 13, wherein the fragment is sufficient to bind its target, e.g., AF9, and inhibit expression and/or activity of SEC.

In one embodiment, an anti-SEC antibody or antibody reagent is conjugated to an E3 ubiquitin ligase recruitment element. In one embodiment, the anti-SEC antibody or antibody reagent conjugated to an E3 ubiquitin ligase recruitment element further comprises a linker.

In one embodiment, the agent that inhibits the SEC is an antisense oligonucleotide. In one embodiment, the agent that inhibits the SEC is an antisense oligonucleotide that targets at least one component of the SEC. As used herein, an “antisense oligonucleotide” refers to a synthesized nucleic acid sequence that is complementary to a DNA or mRNA sequence, such as that of a microRNA. Antisense oligonucleotides are typically designed to block expression of a DNA or RNA target by binding to the target and halting expression at the level of transcription, translation, or splicing. Antisense oligonucleotides of the present invention are complementary nucleic acid sequences designed to hybridize under cellular conditions to a SEC subunit gene, e.g., CDK9 or AF9. Thus, oligonucleotides are chosen that are sufficiently complementary to the target, i.e., that hybridize sufficiently well and with sufficient specificity in the context of the cellular environment, to give the desired effect. For example, an antisense oligonucleotide that inhibits the SEC may comprise at least 5, at least 10, at least 15, at least 20, at least 25, at least 30, or more bases complementary to a portion of the coding sequence of an SEC subunit e.g., human CDK9 gene (e.g., SEQ ID NO: 9) or the AF9 gene (SEQ ID NO: 6).

In one embodiment, the SEC is depleted from the cell's genome using any genome editing system including, but not limited to, zinc finger nucleases, TALENS, meganucleases, and CRISPR/Cas systems. In one embodiment, at least one component of the SEC is depleted from the cell's genome using any genome editing system including, but not limited to, zinc finger nucleases, TALENS, meganucleases, and CRISPR/Cas systems. In one embodiment, the genomic editing system used to incorporate the nucleic acid encoding one or more guide RNAs into the cell's genome is not a CRISPR/Cas system; this can prevent undesirable cell death in cells that retain a small amount of Cas enzyme/protein. It is also contemplated herein that either the Cas enzyme or the sgRNAs are each expressed under the control of a different inducible promoter, thereby allowing temporal expression of each to prevent such interference.

When a nucleic acid encoding one or more sgRNAs and a nucleic acid encoding an RNA-guided endonuclease each need to be administered in vivo, the use of an adenovirus associated vector (AAV) is specifically contemplated. Other vectors for simultaneously delivering nucleic acids to both components of the genome editing/fragmentation system (e.g., sgRNAs, RNA-guided endonuclease) include lentiviral vectors, such as Epstein Barr, Human immunodeficiency virus (HIV), and hepatitis B virus (HBV). Each of the components of the RNA-guided genome editing system (e.g., sgRNA and endonuclease) can be delivered in a separate vector as known in the art or as described herein.

In one embodiment, the agent inhibits the SEC, e.g., at least one component of the SEC, by RNA inhibition. Inhibitors of the expression of a given gene can be an inhibitory nucleic acid. In some embodiments of any of the aspects, the inhibitory nucleic acid is an inhibitory RNA (iRNA). The RNAi can be single stranded or double stranded.

The iRNA can be siRNA, shRNA, endogenous microRNA (miRNA), or artificial miRNA. In one embodiment, an iRNA as described herein effects inhibition of the expression and/or activity of a target, e.g., at least one SEC component, for example, CDK9 or AF9. In some embodiments of any of the aspects, the agent is siRNA that inhibits at least one component of the SEC. In some embodiments of any of the aspects, the agent is shRNA that inhibits at least one component of the SEC.

One skilled in the art would be able to design siRNA, shRNA, or miRNA to target an SEC subunit, e.g., using publically available design tools. siRNA, shRNA, or miRNA is commonly made using companies such as Dharmacon (Layfayette, CO) or Sigma Aldrich (St. Louis, MO).

In some embodiments of any of the aspects, the iRNA can be a dsRNA. A dsRNA includes two RNA strands that are sufficiently complementary to hybridize to form a duplex structure under conditions in which the dsRNA will be used. One strand of a dsRNA (the antisense strand) includes a region of complementarity that is substantially complementary, and generally fully complementary, to a target sequence. The target sequence can be derived from the sequence of an mRNA formed during the expression of the target. The other strand (the sense strand) includes a region that is complementary to the antisense strand, such that the two strands hybridize and form a duplex structure when combined under suitable conditions

The RNA of an iRNA can be chemically modified to enhance stability or other beneficial characteristics. The nucleic acids featured in the invention may be synthesized and/or modified by methods well established in the art, such as those described in “Current protocols in nucleic acid chemistry,” Beaucage, S. L. et al. (Edrs.), John Wiley & Sons, Inc., New York, NY, USA, which is hereby incorporated herein by reference.

In one embodiment, the agent is miRNA that inhibits the SEC, i.e., at least one component of the SEC. microRNAs are small non-coding RNAs with an average length of 22 nucleotides. These molecules act by binding to complementary sequences within mRNA molecules, usually in the 3′ untranslated (3′UTR) region, thereby promoting target mRNA degradation or inhibited mRNA translation. The interaction between microRNA and mRNAs is mediated by what is known as the “seed sequence”, a 6-8-nucleotide region of the microRNA that directs sequence-specific binding to the mRNA through imperfect Watson-Crick base pairing. More than 900 microRNAs are known to be expressed in mammals. Many of these can be grouped into families on the basis of their seed sequence, thereby identifying a “cluster” of similar microRNAs. A miRNA can be expressed in a cell, e.g., as naked DNA. A miRNA can be encoded by a nucleic acid that is expressed in the cell, e.g., as naked DNA or can be encoded by a nucleic acid that is contained within a vector.

The agent may result in gene silencing of the target gene (e.g., the CDK9 component of the SEC), such as with an RNAi molecule (e.g. siRNA or miRNA). This entails a decrease in the mRNA level in a cell for a target by at least about 5%, about 10%, about 20%, about 30%, about 40%, about 50%, about 60%, about 70%, about 80%, about 90%, about 95%, about 99%, or more of the mRNA level found in the cell without the presence of the agent. In one preferred embodiment, the mRNA levels are decreased by at least about 70%, about 80%, about 90%, about 95%, about 99%, or more as compared to the levels in a cell without the presence of the agent. One skilled in the art will be able to readily assess whether the siRNA, shRNA, or miRNA effective target e.g., at least one component of the SEC, such as CDK9, for its downregulation, for example by transfecting the siRNA, shRNA, or miRNA into cells and detecting the levels of a gene (e.g., CDK9) found within the cell via western-blotting.

The agent may be contained in and thus further include a vector. Many such vectors useful for transferring exogenous genes into target mammalian cells are available. The vectors may be episomal, e.g. plasmids, virus-derived vectors such cytomegalovirus, adenovirus, etc., or may be integrated into the target cell genome, through homologous recombination or random integration, e.g. retrovirus-derived vectors such as MMLV, HIV-1, ALV, etc. In some embodiments, combinations of retroviruses and an appropriate packaging cell line may also find use, where the capsid proteins will be functional for infecting the target cells. Usually, the cells and virus will be incubated for at least about 24 hours in the culture medium. The cells are then allowed to grow in the culture medium for short intervals in some applications, e.g. 24-73 hours, or for at least two weeks, and may be allowed to grow for five weeks or more, before analysis. Commonly used retroviral vectors are “defective”, i.e. unable to produce viral proteins required for productive infection. Replication of the vector requires growth in the packaging cell line.

The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, artificial chromosome, virus, virion, etc.

As used herein, the term “expression vector” refers to a vector that directs expression of an RNA or polypeptide (e.g., an agent that inhibits the SEC) from nucleic acid sequences contained therein linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification. The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g. 5′ untranslated (5′UTR) or “leader” sequences and 3′ UTR or “trailer” sequences, as well as intervening sequences (introns) between individual coding segments (exons).

Integrating vectors have their delivered RNA/DNA permanently incorporated into the host cell chromosomes. Non-integrating vectors remain episomal which means the nucleic acid contained therein is never integrated into the host cell chromosomes. Examples of integrating vectors include retroviral vectors, lentiviral vectors, hybrid adenoviral vectors, and herpes simplex viral vector.

One example of a non-integrative vector is a non-integrative viral vector. Non-integrative viral vectors eliminate the risks posed by integrative retroviruses, as they do not incorporate their genome into the host DNA. One example is the Epstein Barr oriP/Nuclear Antigen-1 (“EBNA1”) vector, which is capable of limited self-replication and known to function in mammalian cells. As containing two elements from Epstein-Barr virus, oriP and EBNA1, binding of the EBNA1 protein to the virus replicon region oriP maintains a relatively long-term episomal presence of plasmids in mammalian cells. This particular feature of the oriP/EBNA1 vector makes it ideal for generation of integration-free iPSCs. Another non-integrative viral vector is adenoviral vector and the adeno-associated viral (AAV) vector.

Another non-integrative viral vector is RNA Sendai viral vector, which can produce protein without entering the nucleus of an infected cell. The F-deficient Sendai virus vector remains in the cytoplasm of infected cells for a few passages, but is diluted out quickly and completely lost after several passages (e.g., 10 passages).

Another example of a non-integrative vector is a minicircle vector. Minicircle vectors are circularized vectors in which the plasmid backbone has been released leaving only the eukaryotic promoter and cDNA(s) that are to be expressed.

As used herein, the term “viral vector” refers to a nucleic acid vector construct that includes at least one element of viral origin and has the capacity to be packaged into a viral vector particle. The viral vector can contain a nucleic acid encoding a polypeptide as described herein in place of non-essential viral genes. The vector and/or particle may be utilized for the purpose of transferring nucleic acids into cells either in vitro or in vivo. Numerous forms of viral vectors are known in the art.

### Compositions

Provided herein is a composition comprising any agent that inhibits the SEC through any of its components described herein.

In one embodiment, the composition further comprises a pharmaceutically acceptable carrier. The phrase “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically acceptable carrier” as used herein means a pharmaceutically acceptable material, composition or vehicle, such as a liquid or solid filler, diluent, excipient, solvent, media, encapsulating material, manufacturing aid (e.g., lubricant, talc magnesium, calcium or zinc stearate, or steric acid), or solvent encapsulating material, involved in maintaining the stability, solubility, or activity of, an agent that inhibits the SEC as described herein. Each carrier must be “acceptable” in the sense of being compatible with the other ingredients of the formulation and not injurious to the patient. The terms “excipient,” “carrier,” “pharmaceutically acceptable carrier” or the like are used interchangeably herein.

Further, provided herein is a use of any composition described herein for the treatment of cancer.

### Administration

In some aspects, the methods described herein relate to treating a subject having or diagnosed as having cancer (e.g., blood cancer) comprise administering an agent that inhibits the SEC, e.g., via inhibition of at least one component of the SEC, as described herein.

Subjects having cancer can be identified by a physician using current methods of diagnosing a condition. Symptoms and/or complications of cancer, which characterize this disease and aid in diagnosis are well known in the art. Tests that may aid in a diagnosis of, e.g., cancer, include blood tests and non-invasive imaging. A family history of a particular cancer will also aid in determining if a subject is likely to have the condition or in making a diagnosis of cancer.

Administration of an agent described herein (e.g., an agent that inhibits the SEC) can be performed in a variety of manners, for example, in a single dose, in reoccurring multiple doses, via continuous infusion, via pulsed administration. In one embodiment, an agent described herein can be administered to a subject at least once every 1, 2, 3, 4, 5, 6, 7, 8, 9, 10, 11, 12, 13, 14, 15, 16, 17, 18, 19, 20, 21, 22, 23, or 24 hours; or every 1, 2, 3, 4, 5, 6, or 7 days; or every 1, 2, 3, or 4 weeks; or every 1, 2, 3, 4, 5, 6, 7, 8, 9, 10, 11, or 12 months, or more. It is specifically contemplated herein that the dosing of an agent described herein is determined based on the half-life of the agent, e.g., such that the effect of the agent described herein is continuous, or nearly continuous, in the subject. For example, if the half-life of a given agent that inhibits the SEC is 12 hours, it would be administered every 12 hours to the subject such that it maintains continuous inhibition of the SEC in the subject.

In one embodiment, the agent described herein is administered at least once. In one embodiment, the agent described herein is administered at least twice. For example, the agent described herein is administered at least 2, 3, 4, 5, 6, 7, 8, 9, 10, 11, 12, 13, 14, 15, 16, 17, 18, 19, 20, or more times.

In some embodiments, the methods described herein comprise administering an effective amount of the agent to a subject in order to alleviate at least one symptom of a given cancer. As used herein, “alleviating at least one symptom of a given cancer” is ameliorating any condition or symptom associated with cancer. As compared with an equivalent untreated control, such reduction is by at least 5%, 10%, 20%, 40%, 50%, 60%, 80%, 90%, 95%, 99% or more as measured by any standard technique. A variety of means for administering the agent described herein to subjects are known to those of skill in the art. In one embodiment, the agent is administered systemically or locally (e.g., to the affected organ, e.g., the colon). In one embodiment, the agent is administered intravenously. In one embodiment, the agent is administered continuously, in intervals, or sporadically. The route of administration of the agent will be optimized for the type of agent being delivered (e.g., an antibody, a small molecule, an RNAi), and can be determined by a skilled practitioner.

The term “effective amount” as used herein refers to the amount of an agent described herein that can be administered to a subject having or diagnosed as having cancer (e.g., a blood cancer) needed to alleviate at least one or more symptom of cancer. The term “therapeutically effective amount” therefore refers to an amount of an agent that is sufficient to provide a particular anti-cancer effect when administered to a typical subject. An effective amount as used herein, in various contexts, would also include an amount of an agent sufficient to delay the development of a symptom of cancer, alter the course of a symptom of cancer (e.g., slowing the progression of cancer), or reverse a symptom of cancer. Thus, it is not generally practicable to specify an exact “effective amount”. However, for any given case, an appropriate “effective amount” can be determined by one of ordinary skill in the art using only routine experimentation.

In one embodiment, the agent is administered continuously (e.g., at constant levels over a period of time). Continuous administration of an agent can be achieved, e.g., by epidermal patches, continuous release formulations, or on-body injectors.

Effective amounts, toxicity, and therapeutic efficacy can be evaluated by standard pharmaceutical procedures in cell cultures or experimental animals. The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a circulating plasma concentration range that includes the IC50 (i.e., the concentration of the agent, which achieves a half-maximal inhibition of symptoms) as determined in cell culture, or in an appropriate animal model. Levels in plasma can be measured, for example, by high performance liquid chromatography. The effects of any particular dosage can be monitored by a suitable bioassay, e.g., measuring neurological function, or blood work, among others. The dosage can be determined by a physician and adjusted, as necessary, to suit observed effects of the treatment.

### Dosage

“Unit dosage form” as the term is used herein refers to a dosage suitable for one administration. By way of example, a unit dosage form can be an amount of therapeutic disposed in a delivery device, e.g., a syringe or intravenous drip bag. In one embodiment, a unit dosage form is administered in a single administration. In another, embodiment more than one unit dosage form can be administered simultaneously.

Typically, the dosage ranges are between 0.001 mg/kg body weight to 5 g/kg body weight, inclusive. In some embodiments, the dosage range is from 0.001 mg/kg body weight to 1 g/kg body weight, from 0.001 mg/kg body weight to 0.5 g/kg body weight, from 0.001 mg/kg body weight to 0.1 g/kg body weight, from 0.001 mg/kg body weight to 50 mg/kg body weight, from 0.001 mg/kg body weight to 25 mg/kg body weight, from 0.001 mg/kg body weight to 10 mg/kg body weight, from 0.001 mg/kg body weight to 5 mg/kg body weight, from 0.001 mg/kg body weight to 1 mg/kg body weight, from 0.001 mg/kg body weight to 0.1 mg/kg body weight, from 0.001 mg/kg body weight to 0.005 mg/kg body weight. Alternatively, in some embodiments the dosage range is from 0.1 g/kg body weight to 5 g/kg body weight, from 0.5 g/kg body weight to 5 g/kg body weight, from 1 g/kg body weight to 5 g/kg body weight, from 1.5 g/kg body weight to 5 g/kg body weight, from 2 g/kg body weight to 5 g/kg body weight, from 2.5 g/kg body weight to 5 g/kg body weight, from 3 g/kg body weight to 5 g/kg body weight, from 3.5 g/kg body weight to 5 g/kg body weight, from 4 g/kg body weight to 5 g/kg body weight, from 4.5 g/kg body weight to 5 g/kg body weight, from 4.8 g/kg body weight to 5 g/kg body weight. In one embodiment, the dose range is from 5 μg/kg body weight to 30 μg/kg body weight. Alternatively, the dose range will be titrated to maintain serum levels between 5 μg/mL and 30 μg/mL.

The dosage of the agent that inhibits the SEC; e.g., at least one component of the SEC, as described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to administer further cells, discontinue treatment, resume treatment, or make other alterations to the treatment regimen. The dosage should not be so large as to cause adverse side effects, such as cytokine release syndrome. Generally, the dosage will vary with the age, condition, and sex of the patient and can be determined by one of skill in the art. The dosage can also be adjusted by the individual physician in the event of any complication.

### Combination Treatment

In one aspect, the agent that inhibits the SEC; e.g., at least one component of the SEC, described herein is administered in combination for the treatment of cancer. Administered “in combination,” as used herein, means that two (or more) different treatments (e.g., an agent, or a cancer therapy) are delivered to the subject during the course of the subject's affliction with the disorder, e.g., the two or more treatments are delivered after the subject has been diagnosed with the disorder (e.g., cancer) and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered. The agent described herein and the at least one additional therapy can be administered simultaneously, in the same or in separate compositions, or sequentially. For sequential administration, the agent described herein can be administered first, and the additional agent or anti-cancer treatment can be administered second, or the order of administration can be reversed. The agent and/or other therapeutic agents, procedures or modalities can be administered during periods of active disorder, or during a period of remission or less active disease. The agent can be administered before another treatment, concurrently with the treatment, post-treatment, or during remission of the disorder.

In one embodiment, the cancer therapy is selected from the group consisting of chemotherapy, radiation therapy, chemo-radiation, immunotherapy, surgery, hormone therapy, stem cell therapy (e.g., hematopoietic stem cell transplant), targeted therapy, gene therapy, chimeric antigen receptor T-cells, immune checkpoint inhibitor, an antibody targeting an antigen on cancer cells, a toxin-conjugated antibody targeting an antigen on cancer cells, and a bispecific antibody that recruits a normal immune cell to a tumor cell by simultaneously binding antigens expressed on each of these cells, and precision therapy.

In other embodiments of any method described herein, the cancer therapy is selected from the group consisting of growth inhibitory agents, cytotoxic agents, anti-angiogenesis agents, apoptotic agents, anti-tubulin agents, anti-HER-2 antibodies, anti-CD20 antibodies, an epidermal growth factor receptor (EGFR) antagonist, a HER1/EGFR inhibitor, a platelet derived growth factor inhibitor, a COX-2 inhibitor, an interferon, and a cytokine (e.g., G-CSF, granulocyte—colony stimulating factor).

In other embodiments, the anti-cancer therapy is selected from the group consisting of 13-cis-retinoic acid, 2-CdA, 2-Chlorodeoxyadenosine, 5-Azacitidine, azacytidine, 5-Fluorouracil, 5-FU, 6-Mercaptopurine, 6-MP, 6-TG, 6-Thioguanine, abiraterone acetate, Abraxane, Accutane®, Actinomycin-D, Adriamycin®, Adrucil®, Afinitor®, Agrylin®, Ala-Cort®, Aldesleukin, Alemtuzumab, ALIMTA, Alitretinoin, Alkaban-AQ®, Alkeran®, All-transretinoic Acid, Alpha Interferon, Altretamine, Amethopterin, Amifostine, Aminoglutethimide, Anagrelide, Anandron®, Anastrozole, Arabinosylcytosine, Ara-C, Aranesp®, Aredia®, Arimidex®, Aromasin®, Arranon®, Arsenic Trioxide, Arzerra™, Asparaginase, ATRA, Avastin®, Axitinib, Azacitidine, BCG, BCNU, Bendamustine, Bevacizumab, Bexarotene, BEXXAR®, Bicalutamide, BiCNU, Blenoxane®, Bleomycin, Bortezomib, Busulfan, Busulfex®, C225, Cabazitaxel, Calcium Leucovorin, Campath® Camptosar® Camptothecin-11, Capecitabine, Caprelsa® Carac™ Carboplatin, Carmustine, Carmustine Wafer, Casodex®, CC-5013, CCI-779, CCNU, CDDP, CeeNU, Cerubidine®, Cetuximab, Chlorambucil, Cisplatin, Citrovorum Factor, Cladribine, Cortisone, Cosmegen®, CPT-11, Crizotinib, Cyclophosphamide, Cytadren®, Cytarabine, Cytarabine Liposomal, Cytosar-U®, Cytoxan®, Dacarbazine, Dacogen, Dactinomycin, Darbepoetin Alfa, Dasatinib, Daunomycin, Daunorubicin, Daunorubicin Hydrochloride, Daunorubicin Liposomal, DaunoXome®, Decadron, Decitabine, Delta-Cortef®, Deltasone®, Denileukin Diftitox, Denosumab, DepoCyt™, Dexamethasone, Dexamethasone Acetate, Dexamethasone Sodium Phosphate, Dexasone, Dexrazoxane, DHAD, DIC, Diodex, Docetaxel, Doxil®, Doxorubicin, Doxorubicin Liposomal, Droxia™, DTIC, DTIC-Dome®, Duralone®, Eculizumab, Efudex®, Eligard™, Ellence™, Eloxatin™, Elspar®, Emcyt®, Epirubicin, Epoetin Alpha, Erbitux, Eribulin, Erlotinib, Erwinia L-asparaginase, Estramustine, Ethyol, Etopophos®, Etoposide, Etoposide Phosphate, Eulexin®, Everolimus, Evista®, Exemestane, Fareston®, Faslodex®, Femara®, Filgrastim, Floxuridine, Fludara®, Fludarabine, Fluoroplex®, Fluorouracil, Fluorouracil (cream), Fluoxymesterone, Flutamide, Folinic Acid, FUDR®, Fulvestrant, Gefitinib, Gemcitabine, Gemtuzumab ozogamicin, Gemzar, Gleevec™, Gliadel® Wafer, Goserelin, Granulocyte-Colony Stimulating Factor (G-CSF), Granulocyte Macrophage Colony Stimulating Factor (GM-CSF), Halaven®, Halotestin®, Herceptin®, Hexadrol, Hexalen®, Hexamethylmelamine, HMM, Hycamtin®, Hydrea®, Hydrocort Acetate®, Hydrocortisone, Hydrocortisone Sodium Phosphate, Hydrocortisone Sodium Succinate, Hydrocortone Phosphate, Hydroxyurea, Ibritumomab, Ibritumomab Tiuxetan, Idamycin®, Idarubicin, Ifex®, IFN-alpha, Ifosfamide, IL-11, IL-2, Imatinib mesylate, Imidazole Carboxamide, Inlyta®, Interferon alpha, Interferon Alpha-2b (PEG Conjugate), Interleukin-2, Interleukin-11, Intron A® (interferon alpha-2b), Ipilimumab, Iressa®, Irinotecan, Isotretinoin, Ixabepilone, Ixempra™, Jevtana®, Kidrolase (t), Lanacort®, Lapatinib, L-asparaginase, LCR, Lenalidomide, Letrozole, Leucovorin, Leukeran, Leukine™, Leuprolide, Leurocristine, Leustatin™, Liposomal Ara-C, Liquid Pred®, Lomustine, L-PAM, L-Sarcolysin, Lupron®, Lupron Depot®, Matulane®, Maxidex, Mechlorethamine, Mechlorethamine Hydrochloride, Medralone®, Medrol®, Megace®, Megestrol, Megestrol Acetate, Melphalan, Mercaptopurine, Mesna, Mesnex™, Methotrexate, Methotrexate Sodium, Methylprednisolone, Meticorten®, Mitomycin, Mitomycin-C, Mitoxantrone, M-Prednisol®, MTC, MTX, Mustargen®, Mustine, Mutamycin®, Myleran®, Mylocel™, Mylotarg®, Navelbine®, Nelarabine, Neosar®, Neulasta™, Neumega®, Neupogen®, Nexavar®, Nilandron®, Nilotinib, Nilutamide, Nipent®, Nitrogen Mustard, Novaldex®, Novantrone®, Nplate, Octreotide, Octreotide acetate, Ofatumumab, Oncospar®, Oncovin®, Ontak®, Onxal™, Oprelvekin, Orapred®, Orasone®, Oxaliplatin, Paclitaxel, Paclitaxel Protein-bound, Pamidronate, Panitumumab, Panretin®, Paraplatin®, Pazopanib, Pediapred®, PEG Interferon, Pegaspargase, Pegfilgrastim, PEG-INTRON™, PEG-L-asparaginase, PEMETREXED, Pentostatin, Phenylalanine Mustard, Platinol®, Platinol-AQ®, Prednisolone, Prednisone, Prelone®, Procarbazine, PROCRIT®, Proleukin®, Prolia®, Prolifeprospan 20 with Carmustine Implant, Provenge®, Purinethol®, Raloxifene, Revlimid®, Rheumatrex®, Rituxan®, Rituximab, Roferon-A® (Interferon Alfa-2a), Romiplostim, Rubex®, Rubidomycin hydrochloride, Sandostatin®, Sandostatin LAR®, Sargramostim, Sipuleucel-T, Soliris®, Solu-Cortef®, Solu-Medrol®, Sorafenib, SPRYCEL™, STI-571, Streptozocin, SU11248, Sunitinib, Sutent®, Tamoxifen, Tarceva®, Targretin®, Tasigna®, Taxol®, Taxotere®, Temodar®, Temozolomide, Temsirolimus, Teniposide, TESPA, Thalidomide, Thalomid®, TheraCys®, Thioguanine, Thioguanine Tabloid®, Thiophosphoamide, Thioplex®, Thiotepa, TICE®, Toposar®, Topotecan, Toremifene, Torisel®, Tositumomab, Trastuzumab, Treanda®, Tretinoin, Trexall™, Trisenox®, TSPA, TYKERB®, Valrubicin, Valstar, vandetanib, VCR, Vectibix™, Velban®, Velcade®, Vemurafenib, VePesid®, Vesanoid®, Viadur™, Vidaza®, Vinblastine, Vinblastine Sulfate, Vincasar Pfs®, Vincristine, Vinorelbine, Vinorelbine tartrate, VLB, VM-26, Vorinostat, Votrient, VP-16, Vumon®, Xalkori capsules, Xeloda®, Xgeva®, Yervoy®, Zanosar®, Zelboraf, Zevalin™, Zinecard®, Zoladex®, Zoledronic acid, Zolinza, Zometa®, and Zytiga®.

In one embodiment, an agent that inhibits the SEC is administered with an anti-cancer therapy at a sub-clinical dose, for example, a sub-clinical dose of a chemotherapy. As used herein, “sub-clinical” refers to a dosage or regimen that is not sufficient to elicit a therapeutic effect.

In one embodiment, co-administration of an agent described herein and an anti-cancer therapy allows for administration of the anti-cancer therapy at a lower dose as compared to administration of the anti-cancer therapy as a monotherapy.

### Parenteral Dosage Forms

Parenteral dosage forms of an agent that inhibits the SEC described herein can be administered to a subject by various routes, including, but not limited to, subcutaneous, intravenous (including bolus injection), intramuscular, and intraarterial. Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, controlled-release parenteral dosage forms, and emulsions.

Suitable vehicles that can be used to provide parenteral dosage forms of the disclosure are well known to those skilled in the art. Examples include, without limitation: sterile water; water for injection USP; saline solution; glucose solution; aqueous vehicles such as but not limited to, sodium chloride injection, Ringer's injection, dextrose Injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate.

### Controlled and Delayed Release Dosage Forms

In some embodiments of the aspects described herein, an agent that inhibits the SEC is administered to a subject by controlled- or delayed-release means. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. (Kim, Cherng-ju, Controlled Release Dosage Form Design, 2 (Technomic Publishing, Lancaster, Pa.: 2000)). Controlled-release formulations can be used to control a compound of formula (I)'s onset of action, duration of action, plasma levels within the therapeutic window, and peak blood levels. In particular, controlled- or extended-release dosage forms or formulations can be used to ensure that the maximum effectiveness of an agent is achieved while minimizing potential adverse effects and safety concerns, which can occur both from under-dosing a drug (i.e., going below the minimum therapeutic levels) as well as exceeding the toxicity level for the drug.

A variety of known controlled- or extended-release dosage forms, formulations, and devices can be adapted for use with any agent described herein. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,733,566; and 6,365,185, each of which is incorporated herein by reference in their entireties. These dosage forms can be used to provide slow or controlled-release of one or more active ingredients using, for example, hydroxypropylmethyl cellulose, other polymer matrices, gels, permeable membranes, osmotic systems (such as OROS® (Alza Corporation, Mountain View, Calif USA)), multilayer coatings, microparticles, liposomes, or microspheres or a combination thereof to provide the desired release profile in varying proportions. Additionally, ion exchange materials can be used to prepare immobilized, adsorbed salt forms of the disclosed compounds and thus effect controlled delivery of the drug. Examples of specific anion exchangers include, but are not limited to, DUOLITE® A568 and DUOLITE® AP143 (Rohm&Haas, Spring House, Pa. USA).

### Efficacy

The efficacy of an agent that inhibits the SEC described herein, e.g., for the treatment of cancer, such as a blood cancer, can be determined by the skilled practitioner. However, a treatment is considered “effective treatment,” as the term is used herein, if one or more of the signs or symptoms of cancer are altered in a beneficial manner, other clinically accepted symptoms are improved, or even ameliorated, or a desired response is induced e.g., by at least 10% following treatment according to the methods described herein. Efficacy can be assessed, for example, by measuring a marker, indicator, symptom, and/or the incidence of a condition treated (e.g., cancer) according to the methods described herein or any other measurable parameter appropriate. Efficacy can also be measured by a failure of an individual to worsen as assessed by hospitalization, or need for medical interventions (i.e., progression of cancer). Methods of measuring these indicators are known to those of skill in the art and/or are described herein.

Efficacy can be assessed in animal models of a condition described herein, for example, a mouse model or an appropriate animal model of a given cancer, as the case may be. When using an experimental animal model, efficacy of treatment is evidenced when a statistically significant change in a marker is observed, e.g., a reduction in tumor size, or prevention of metastasis.

All patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The invention described herein can further be described in the following numbered paragraphs:
1. A method for treating cancer, the method comprising administering to a subject having cancer an agent that inhibits the super elongation complex (SEC)
2. The method of paragraph 1, wherein the cancer is a blood cancer.
3. The method of any of the preceding paragraphs, wherein the blood cancer is selected from the group consisting of leukemia, lymphoma, myeloma, and myeloproliferative neoplasms (MPNs)
4. The method of any of the preceding paragraphs, wherein the leukemia is selected from the group consisting of Acute myeloid leukemia (AML), Chronic myeloid leukemia (CML), Acute lymphocytic leukemia (ALL), and Chronic lymphocytic leukemia (CLL).
5. The method of any of the preceding paragraphs, wherein the lymphoma is selected from the group consisting of a non-Hodgkin lymphoma, Hodgkin lymphoma, Diffuse large B-cell lymphoma (DLBCL), Follicular lymphoma, Chronic lymphocytic leukemia (CLL), Small lymphocytic lymphoma (SLL), Mantle cell lymphoma (MCL), Marginal zone lymphomas, and Burkitt lymphoma.
6. The method of any of the preceding paragraphs, wherein the myeloma is selected from the group consisting of multiple myeloma, plasmacytoma, and monoclonal gammopathy of undetermined significance (MGUS).
7. The method of any of the preceding paragraphs, wherein the MPN is selected from the group consisting of Polycythemia vera (PV), Essential thrombocythemia (ET), and Myelofibrosis (MF).
8. The method of any of the preceding paragraphs, wherein the cancer results from or comprises a mutation that impacts hematopoietic stem cell self-renewal
9. The method of any of the preceding paragraphs, wherein the cancer results from or comprises a mutation in the CTR9 gene.
10. The method of any of the preceding paragraphs, where the cancer results from or comprises a mutation in the PAF1 complex.
11. The method of any of the preceding paragraphs, wherein the agent inhibits at least one component of the super elongation complex (SEC).
12. The method of any of the preceding paragraphs, wherein the components of the SEC include Eleven-nineteen lysine-rich leukemia (ELL) 1, ELL2, ELL3, ELL-associated factor (EAF) 1/, EAF2, Mixed-lineage leukemia translocated to chromosome 3 (MLLT3) protein AF-9 (AF9), Mixed-lineage leukemia translocated to chromosome 1 (MLLT1) protein ENL, AF4/FMR2 Family member (AFF) 1, AFF4, and positive transcription elongation factor (P-TEFb).
13. The method of any of the preceding paragraphs, wherein the P-TEFb component is selected from the group consisting of cyclin-dependent kinase 9 (CDK9), cyclin T (CycT) 1, CycT2, CycT3, bromodomain containing 4 protein (BRD4), and 7SK snRNP.
14. The method of any of the preceding paragraphs, wherein the agent that inhibits the SEC is selected from the group consisting of a small molecule inhibitor, a small molecule degrader (proteolysis-targeting chimera, PROTAC), an antibody, a peptide, a genome editing system, an antisense oligonucleotide, and an RNAi.
15. The method of any of the preceding paragraphs, wherein the small molecule is selected from the group consisting of: SR-0813, LDCO000067, AZD4573, atuveciclib, flavopiridol, CR8, indirubin-3′-monoxime derivatives, 5-fluoro-N²,N⁴-diphenylpyrimidine-2,4-diamines, 4-(thiazol-5-ul)-2-(phenylamino)pyrimidines, TG02, CDKI-73, 2,4,5-trisubstited pyrimidine derivatives, Wogonin, PHA-767491, LY2857785, dinaciclib, roscovitine, voruciclib, sns-032, P276-00, FIT-039, CCT068127, MC180295, and KL-1, SR-1114, dTAG13, A-1592668.
16. The method of any of the preceding paragraphs, wherein the RNAi is a microRNA, an siRNA, or a shRNA.
17. The method of any of the preceding paragraphs, wherein the agent that inhibits the SEC is administered at least once.
18. The method of any of the preceding paragraphs, wherein the subject has previously been administered an anti-cancer therapy.
19. The method of any of the preceding paragraphs, wherein the subject has not previously been administered an anti-cancer therapy.
20. The method of any of the preceding paragraphs, further comprising the step of, after the step of administering, administering at least one anti-cancer therapy to the subject.
21. The method of any of the preceding paragraphs, further comprising the step of, prior to the step of administering, administering at least one anti-cancer therapy to the subject.
22. The method of any of the preceding paragraphs, further comprising the step of, after the step of administering, administering low dose chemotherapy to the subject.
23. The method of any of the preceding paragraphs, further comprising the step of, prior to administering, diagnosing a subject as having cancer.
24. The method of any of the preceding paragraphs, further comprising the step of, prior to administering, receiving a result from an assay that diagnoses a subject as having cancer.
25. The method of any of the preceding paragraphs, wherein the anti-cancer treatment is selected from the list consisting of: chemotherapy, hematopoietic stem cell transplant, radiation, chemo-radiation, surgery, chimeric antigen receptor T-cells, immune checkpoint inhibitor, an antibody targeting an antigen on cancer cells, a toxin-conjugated antibody targeting an antigen on cancer cells, and a bispecific antibody that recruits a normal immune cell to a tumor cell by simultaneously binding antigens expressed on each of these cells.
26. A composition comprising an agent that inhibits the SEC.
27. The composition any of the preceding paragraphs, wherein the agent is a small molecule inhibitor, a small molecule degrader (proteolysis-targeting chimera, PROTAC), an antibody, a peptide, a genome editing system, an antisense oligonucleotide, and an RNAi.
28. The composition of any of the preceding paragraphs, further comprising a pharmaceutically acceptable carrier.
29. Use of the composition of any of the preceding paragraphs for the treatment of cancer.

### EXAMPLES

### Example 1

### INTRODUCTION

While the somatic mutations driving diverse blood cancers, including myeloid malignancies, have been well characterized, there is a significant heritable component to these diseases that is poorly understood (1). Common variant association studies (CVAS) have provided valuable insights (2-4), but only identify a fraction of the heritable risk with effect estimates that are typically quite small. With the increasing availability of large-scale sequencing studies and given the impact of rare familial syndromes that predispose to myeloid malignancies (5), rare variant association studies provide a valuable complementary approach to identify additional inherited risk factors for the myeloid malignancies (including the myeloproliferative neoplasms (MPN), acute myeloid leukemia (AML), and myelodysplastic syndromes (MDS)). The overarching concept of data presented herein is that a previously unknown inherited basis for myeloid malignancy predisposition involving the super elongation complex provides an ideal target for therapeutic intervention.

In data presented here, rare variant burden analyses were performed to identify factors predisposing to blood cancers. These data revealed a new mechanism, specifically, misregulation of the super elongation complex (SEC), that results in human hematopoietic stem cell (HSC) expansion and a predisposition to blood cancer. As shown herein variants in CTR9, which encodes a key component of the PAF1 transcription elongation complex results in increased activity of the SEC and subsequently HSC expansion. The super elongation complex (SEC) is a multi-component complex that regulates the elongation stage of transcription. These data indicate that inhibition of expression or activity of the SEC suppress HSC expansion and can prevent or treat blood cancers.

### Research Approach

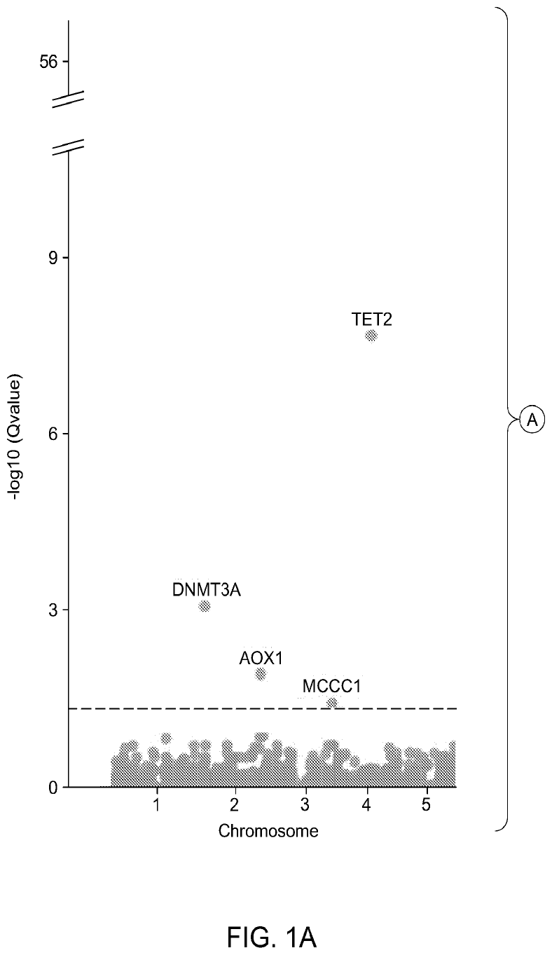

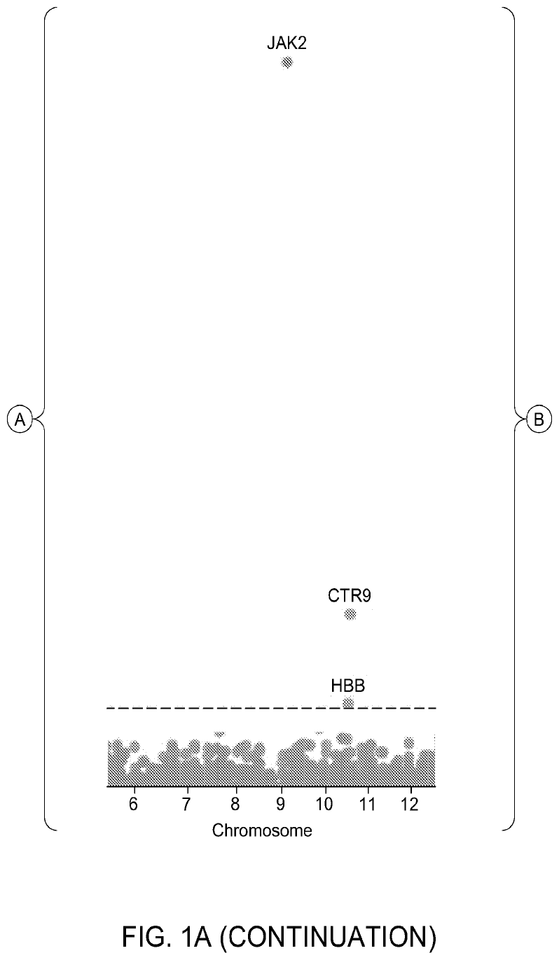

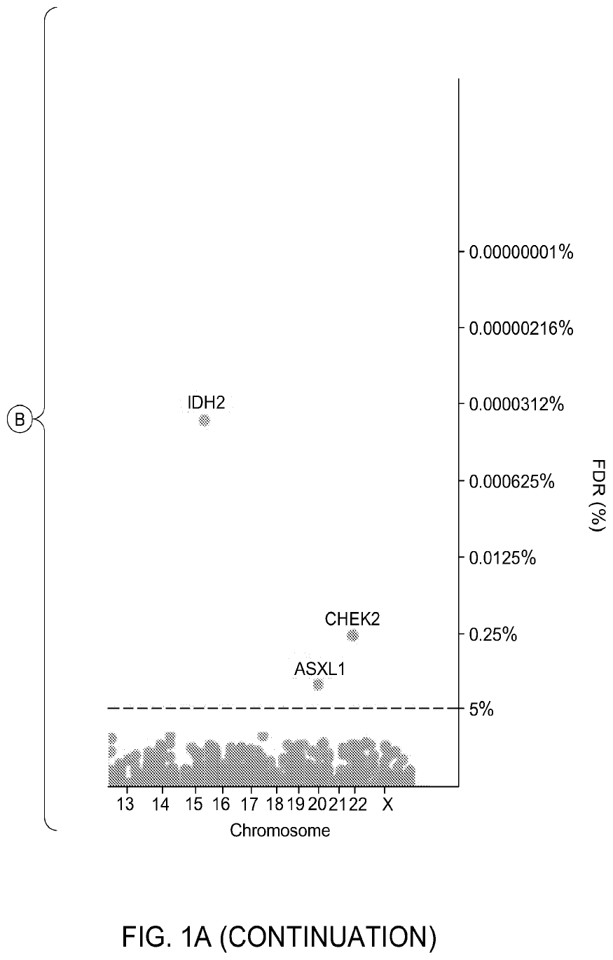

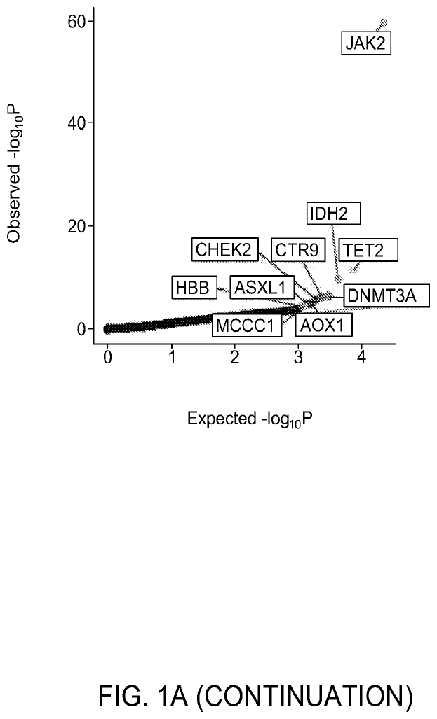

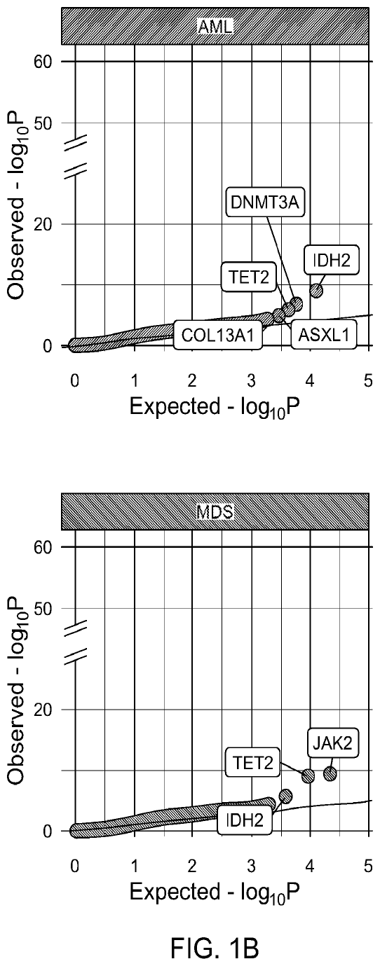

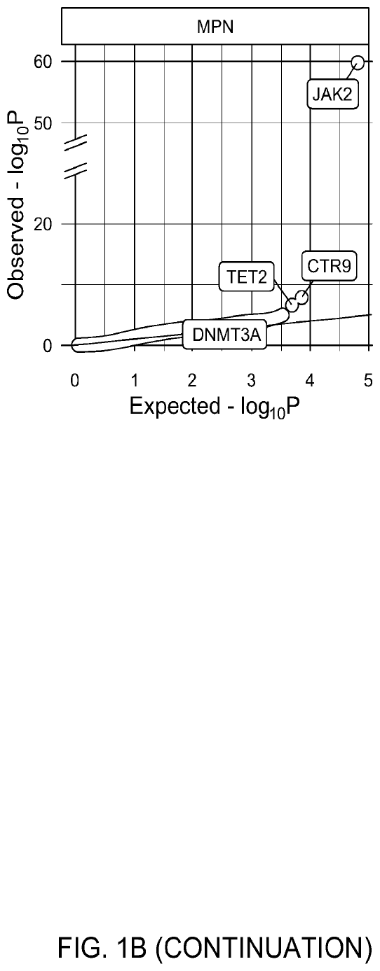

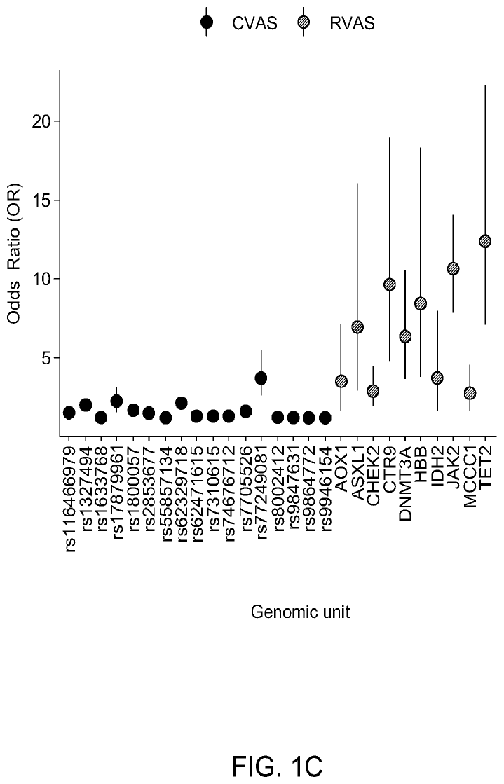

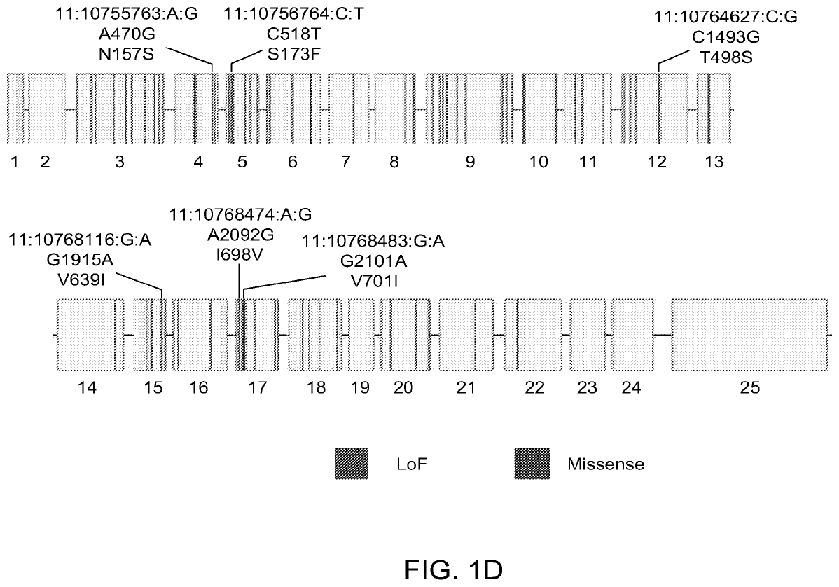

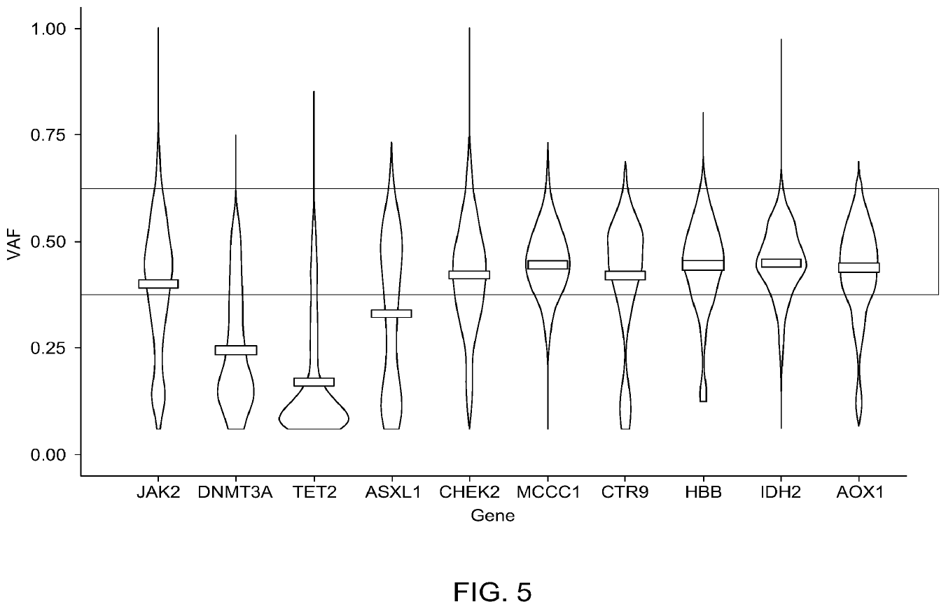

Following QC, 166,953 participants from the UK Biobank and 691,460 rare variants under a frequency of 0.1% and of predicted deleteriousness, were included in a gene-based collapsing association analysis (FIG. 1A). Myeloid malignancies were combined into one phenotype (N_{myeloid}=793 (N_{MPN}=578, N_{AML}=175, N_{MDS}=135), N_{controLs}=166,160), since many risk factors are shared across these blood cancers as well as to increase overall power. Significant burden was seen in a number of genes (JAK2, TET2, IDH2, DNMT3A, ASXL1) known to harbor somatic mutations in clonal hematopoiesis of indeterminate potential (CHIP) (6, 7), and served as positive controls for the assessment of deleterious alleles. Of note, a significant burden in a number of genes including CTR9, CHEK2, AOX1, MCCC1, and HBB was also identified, which showed variant allele fractions in the germline range (FIG. 5) and are typically not seen in CHIP nor as somatic mutations from prior studies of cancer genomes (8).

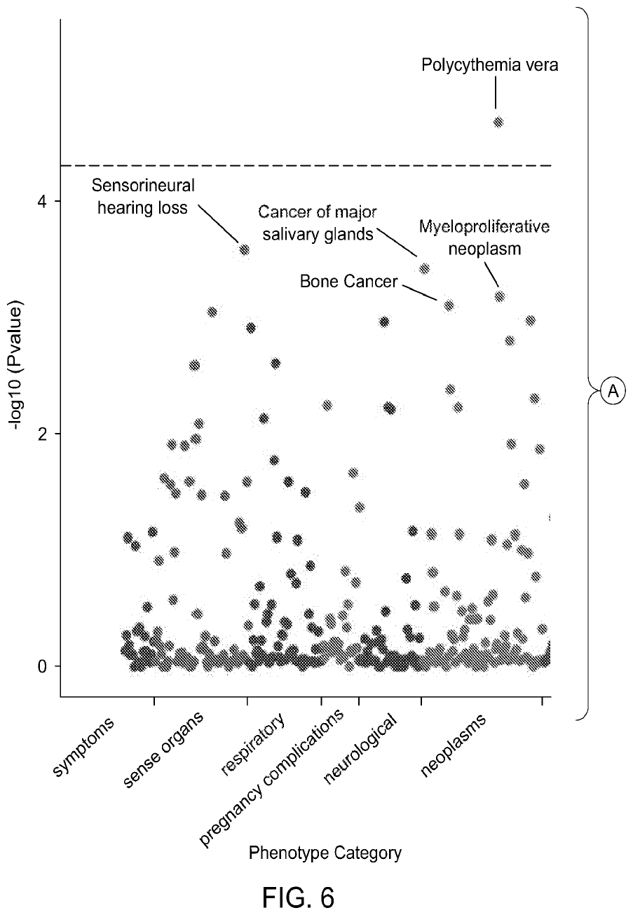

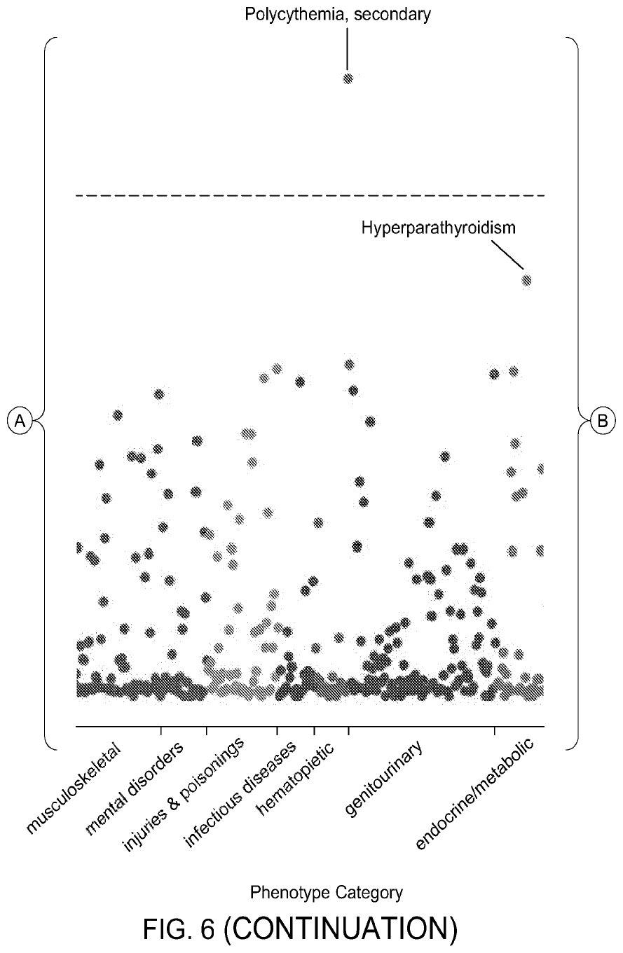

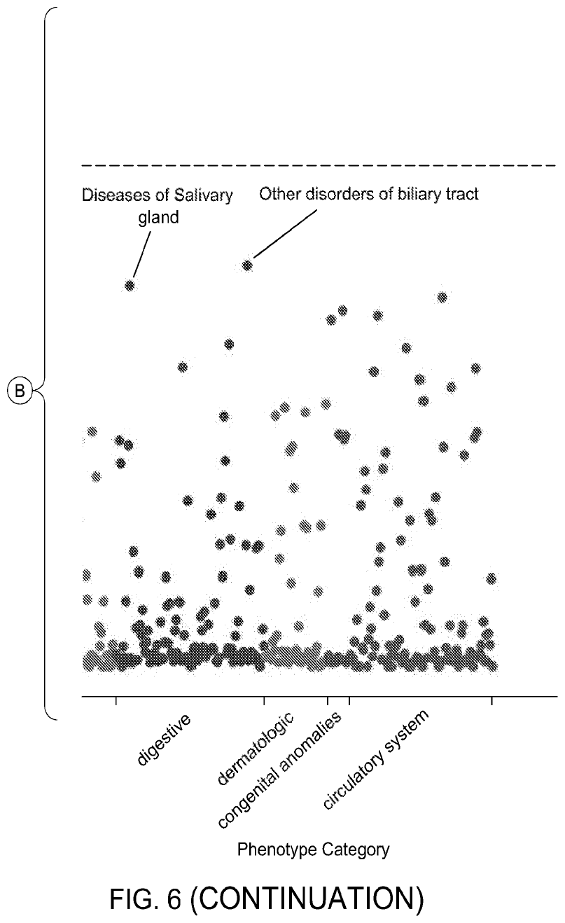

CTR9 was of particular interest and is a key component of the PAF1 complex (PAF1c), which has multiple functions during transcription, including facilitating transcription elongation after promoter-proximal pausing by RNA polymerase II (9, 10). The PAF1c is composed of five subunits: CTR9, CDC73, PAF1, LEO1, and WDR61. Heterozygous loss-of-function mutations in CTR9 have been associated with familial Wilms' tumor, but no other associated cancers have been reported (11). Deleterious mutations in the CDC73 gene increase risk of parathyroid carcinoma (12) and cause the hyperparathyroidism-jaw tumor syndrome (13). Interestingly, PheWAS for CTR9 across 1,027 phenotypes revealed cancer and disease of salivary glands, bone, and hyperparathyroidism as among the most significant, indicating interference with the PAF1c in a manner producing similar phenotypic outcomes to CDC73 heterozygous loss-of-function (FIG. 6).

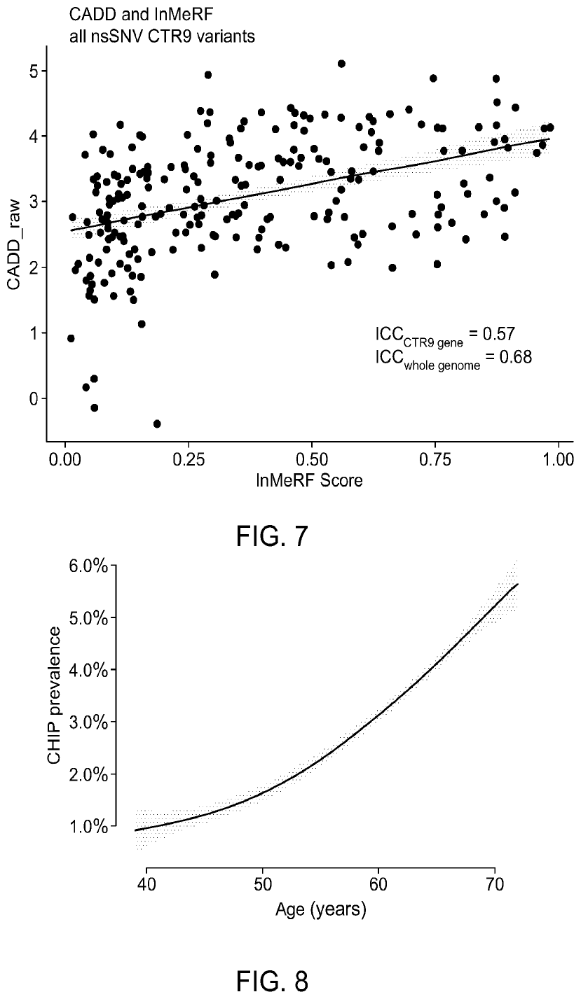

The majority of the signal for CTR9 was driven by MPNs (FIG. 1). As expected, rare variant collapsing tests showed much larger odds ratios (OR) than variants identified from CVAS of myeloid malignancies (FIG. 1C). CTR9 showed a large OR of 9.6 (95% CI=4.86-19.04, SKAT-O p-value=5.47×10⁻⁷) (data not shown). CTR9 germline deleterious variants were not significantly associated with risk of detectable clonal hematopoiesis of indeterminate potential (CHIP) (p=0.17), however there is low sensitivity to identify CHIP due to JAK2 and specific other variants in this cohort (Methods). There were eight cases that carried six deleterious mutations in CTR9, found across multiple exomes of the gene (FIG. 1D). CTR9 rare variant burden was replicated using 211 MPN cases from an independent and previously described cohort (14) against a control set of individuals from gnomAD (n=125,748), p=0.0029 (FIG. 7).

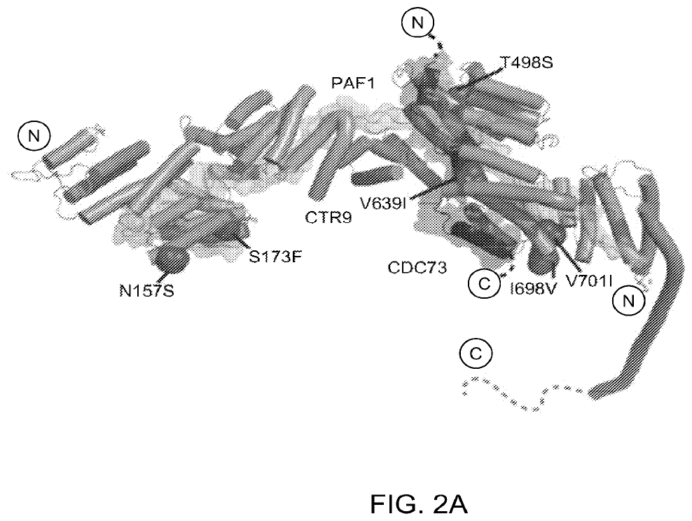

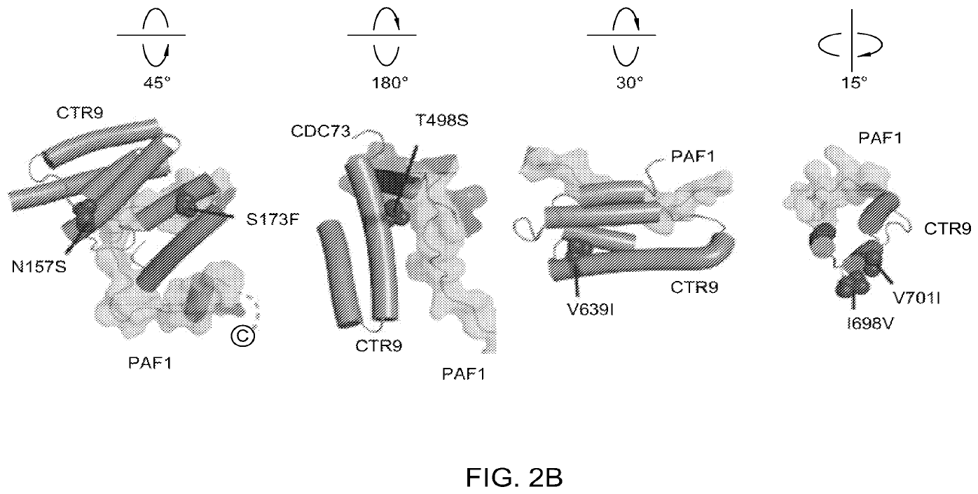

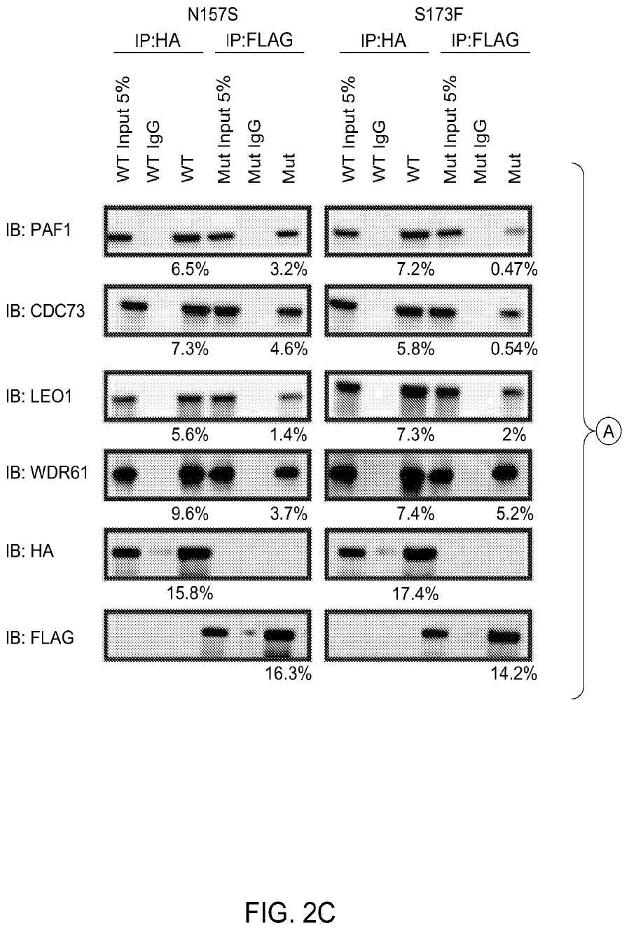

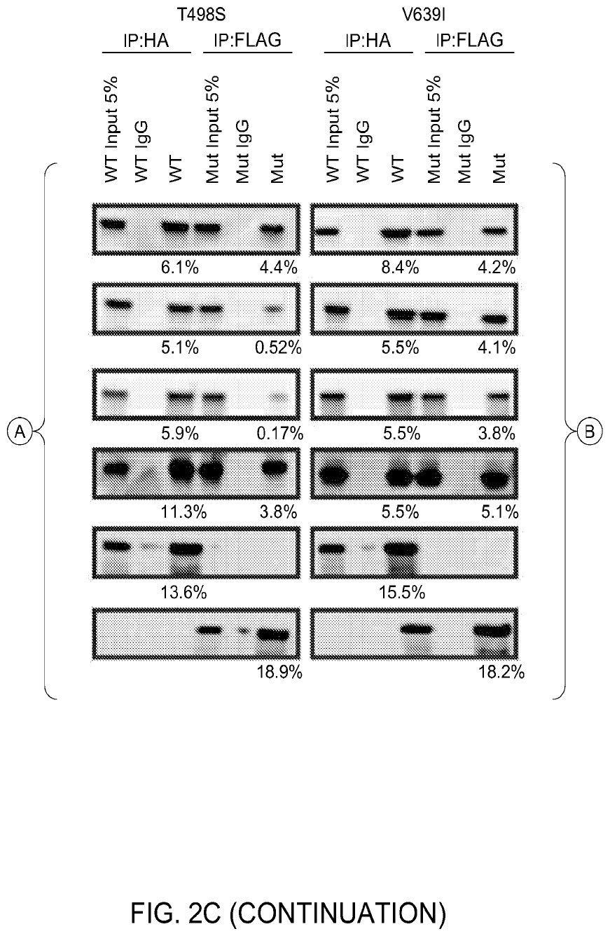

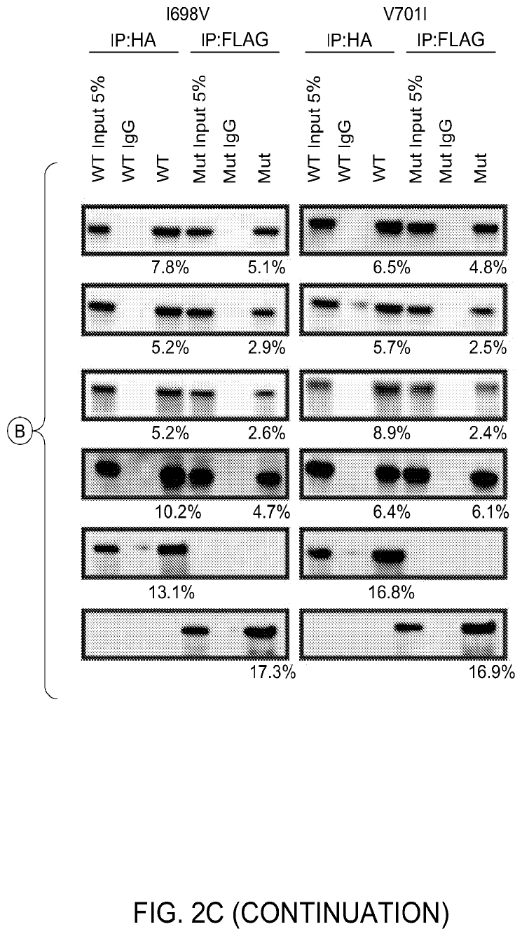

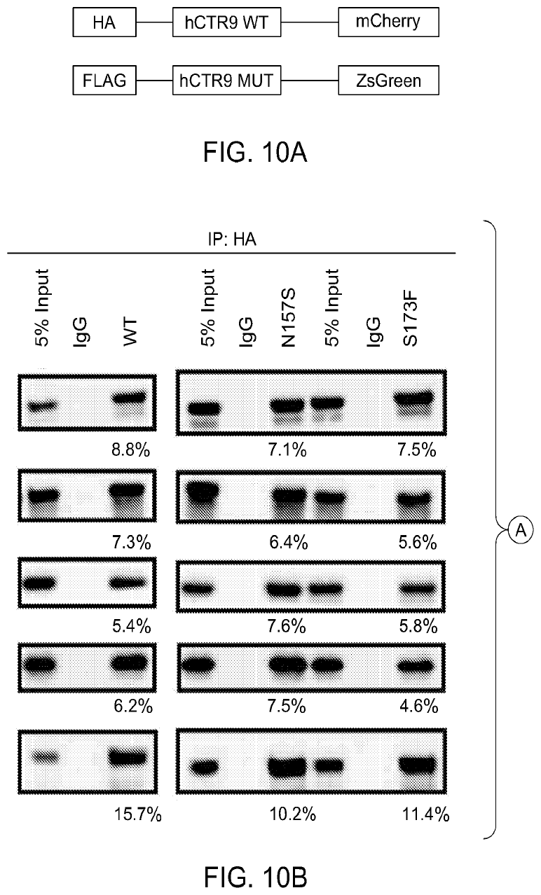

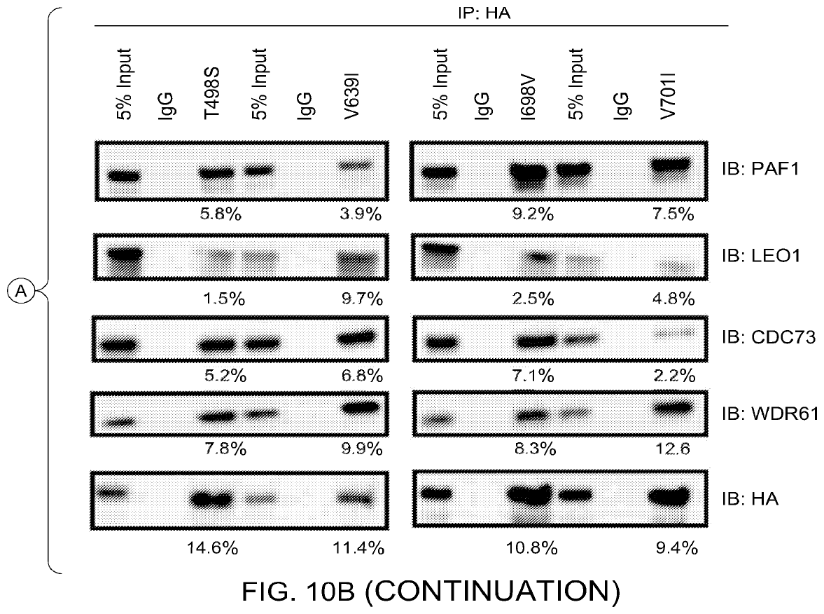

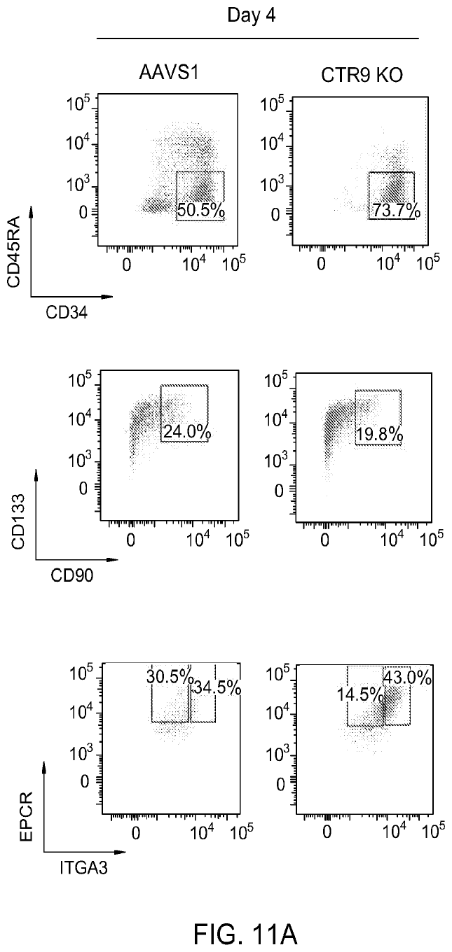

Structural analysis of PAFlc (15, 16) revealed how the deleterious variants in CTR9 found in cases often occur at interfaces involving other key components necessary for transcription. For example, residue 498 is located at the binding interface between CDC73 and PAF1, while residues 698 and 701 are located at the binding interface with subunit RPB8 of RNA polymerase II (FIG. 2A, FIG. 2B) (15, 16). Consistent with the likely disruptive role of these mutations, when expressed as a tagged protein along with wildtype CTR9, reduced interactions with other PAF1c components was inconsistently served, demonstrating loss-of-function by all of these variants (FIG. 2C). Crucially, similar binding by wildtype CTR9 was observed in all of these cases, demonstrating that no mutations were acting in a dominant negative manner, at least in terms of PAF1c assembly (FIG. 10B).

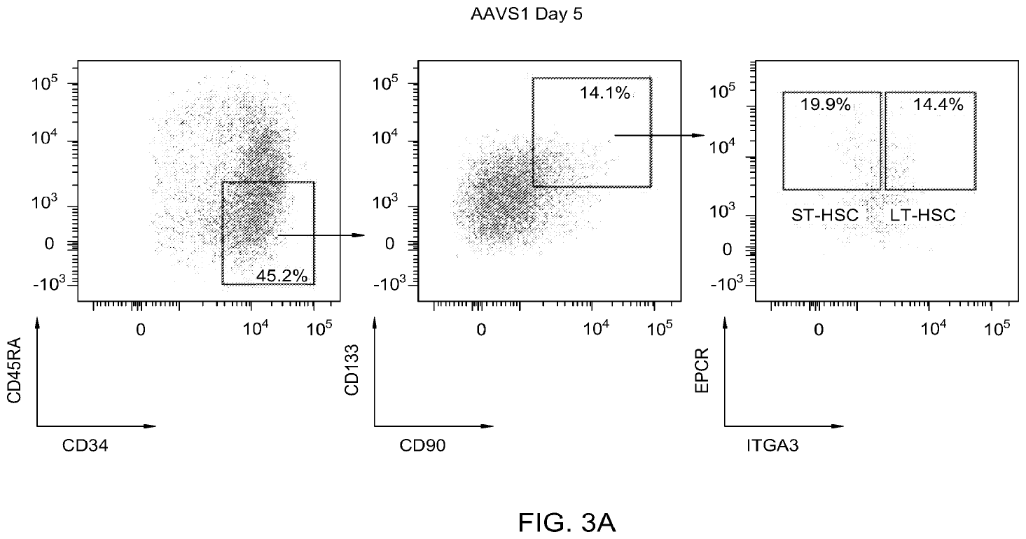

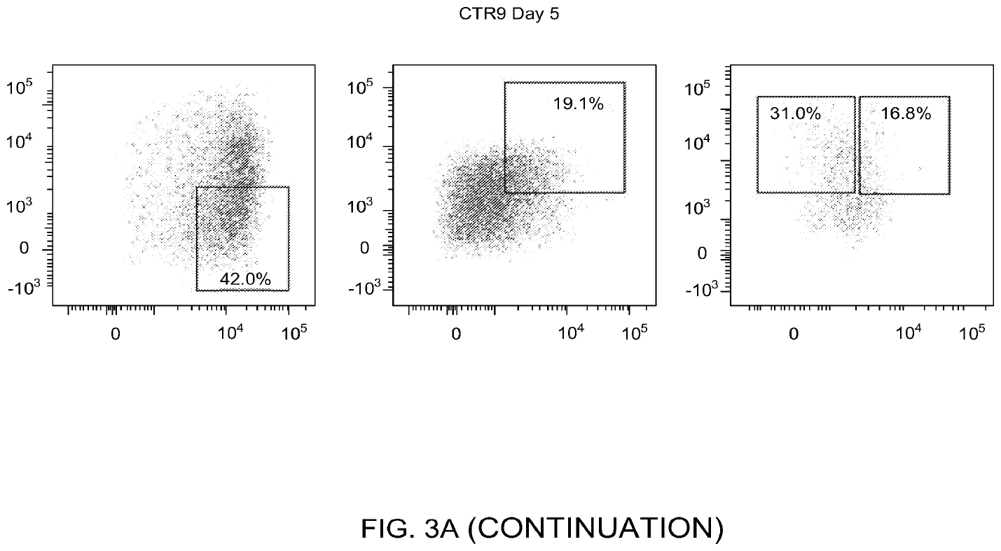

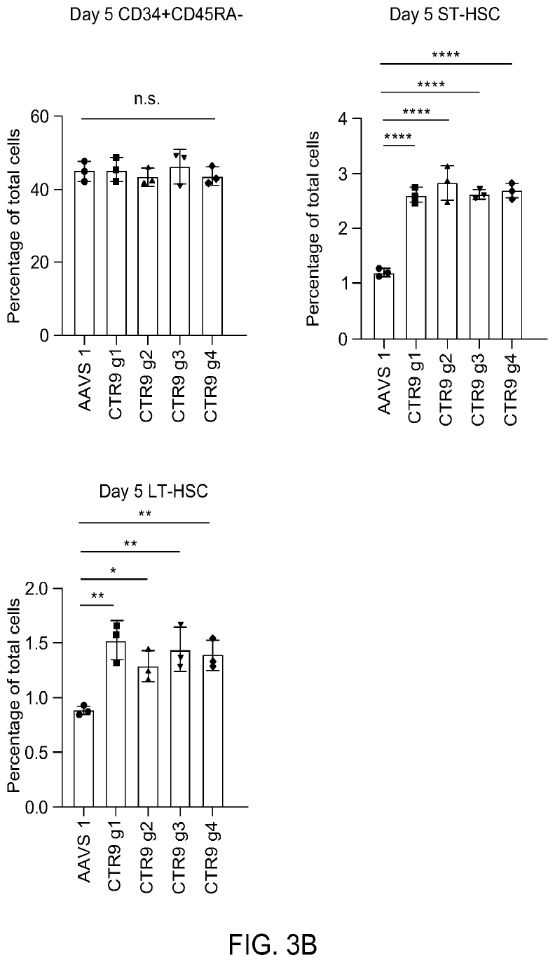

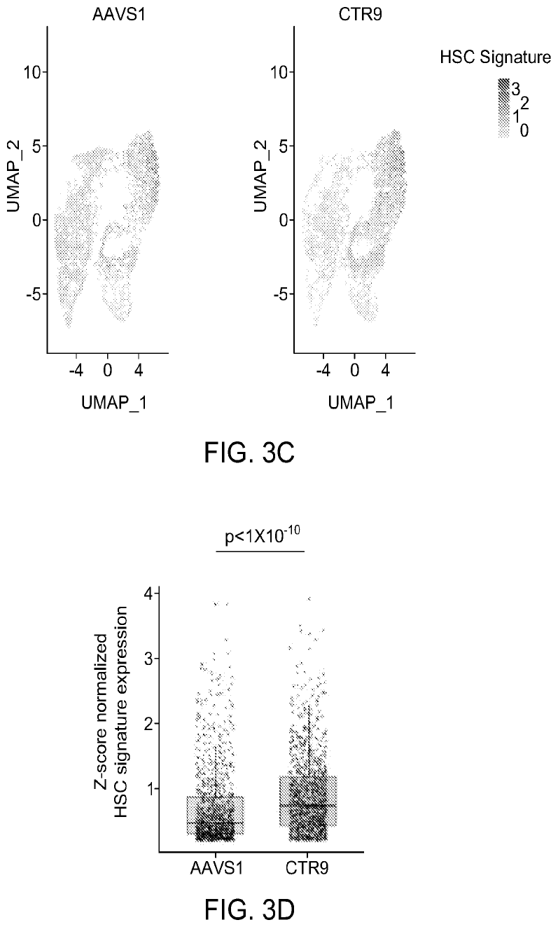

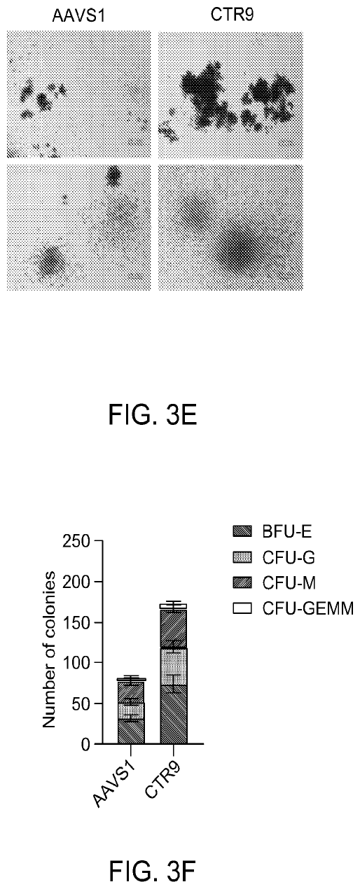

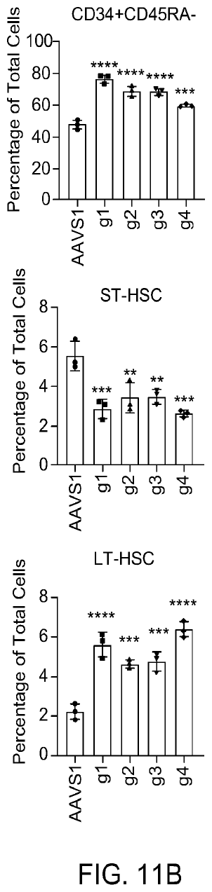

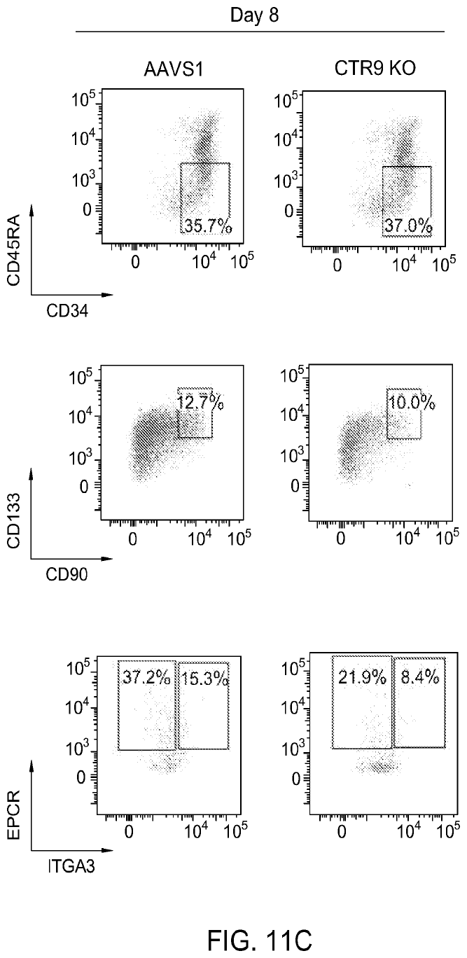

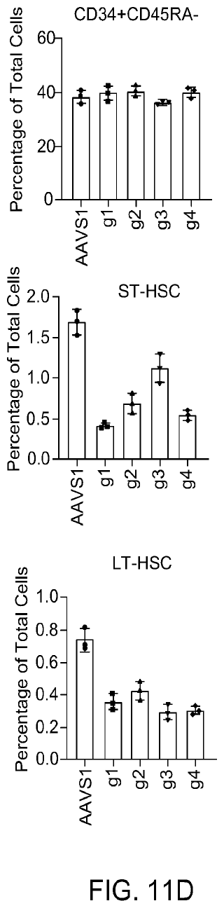

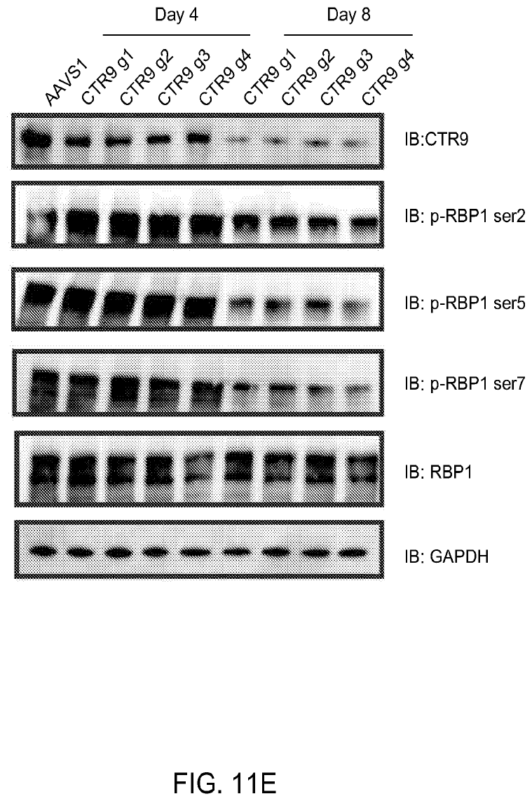

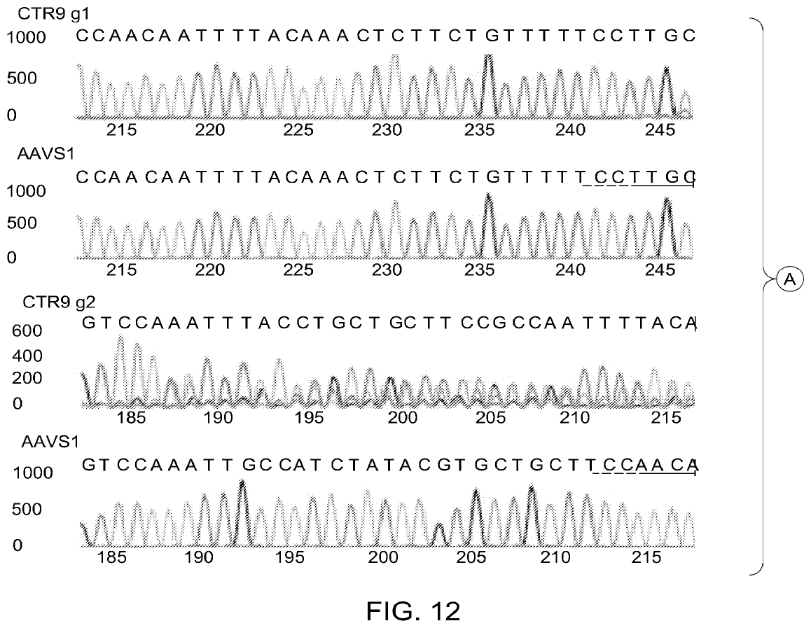

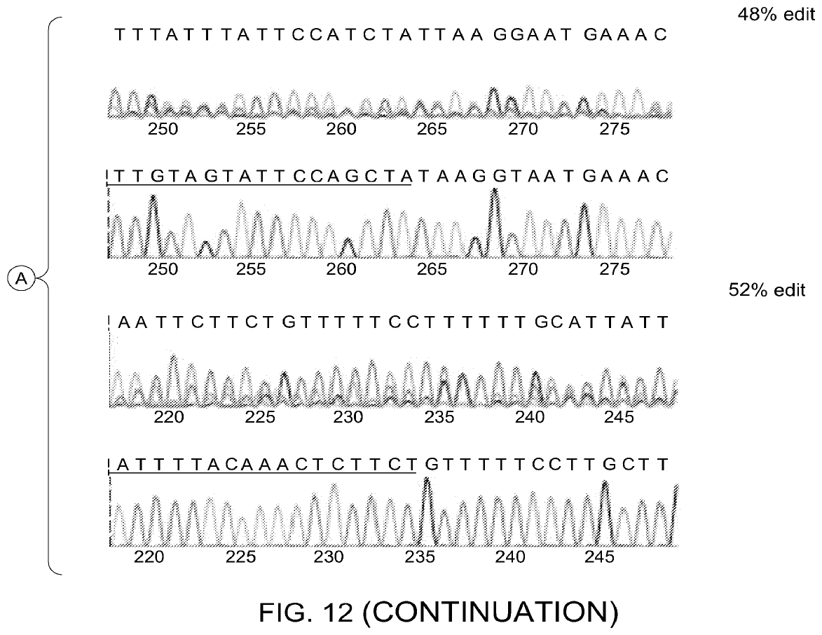

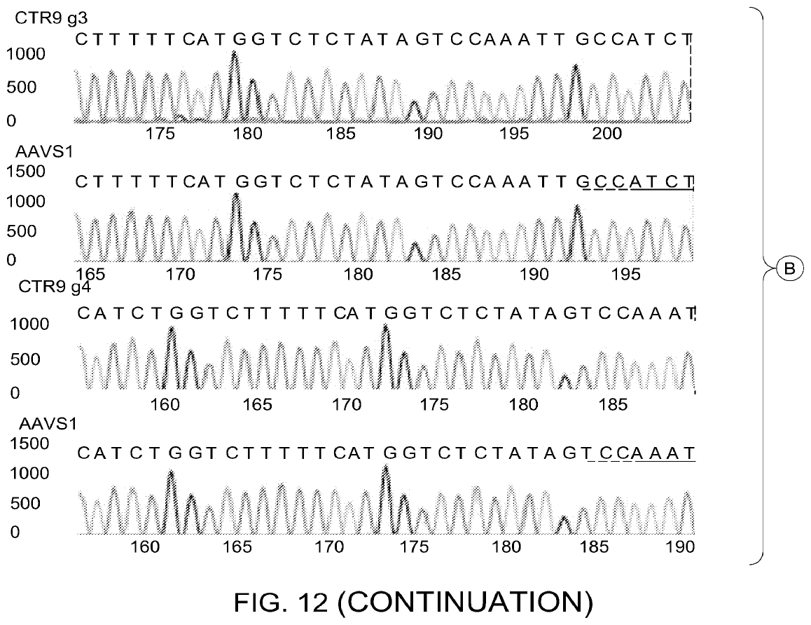

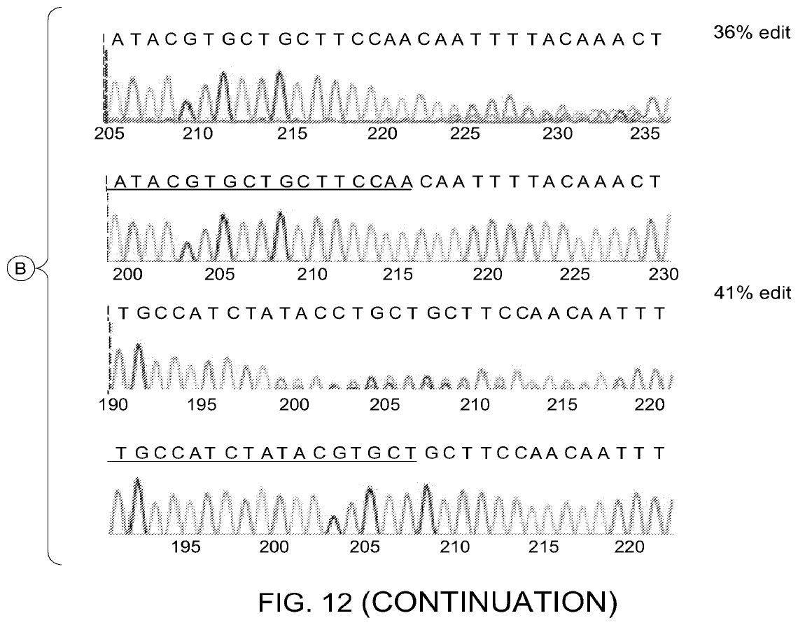

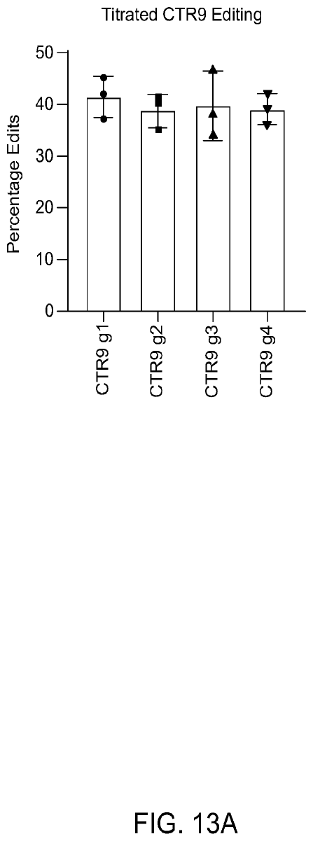

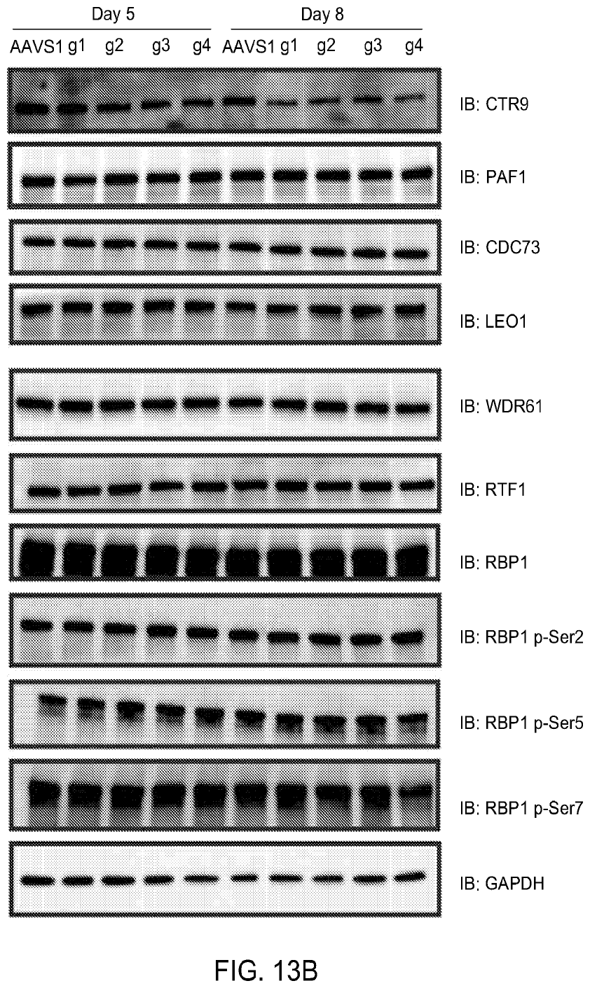

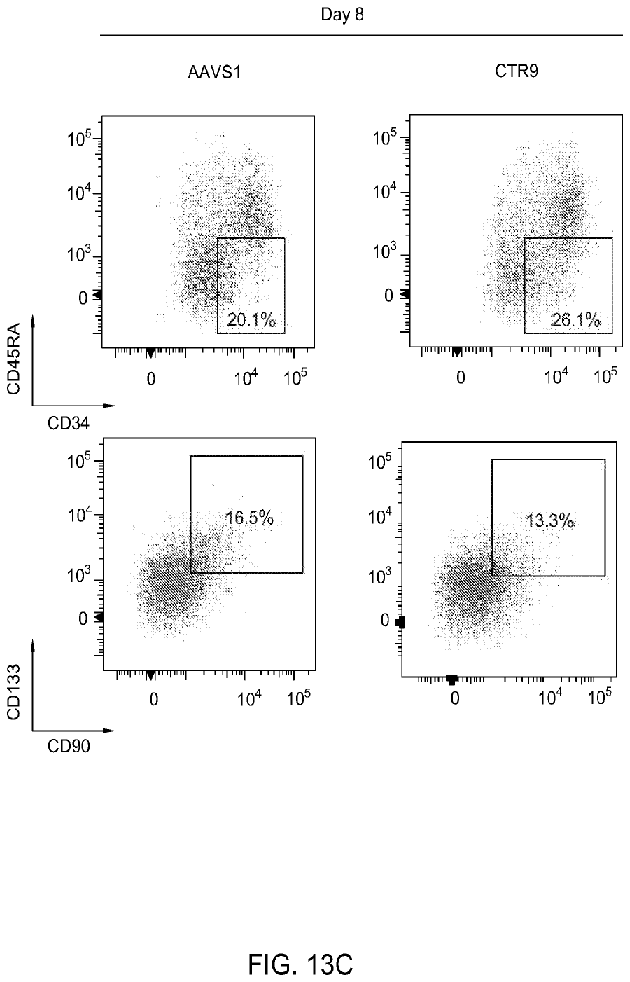

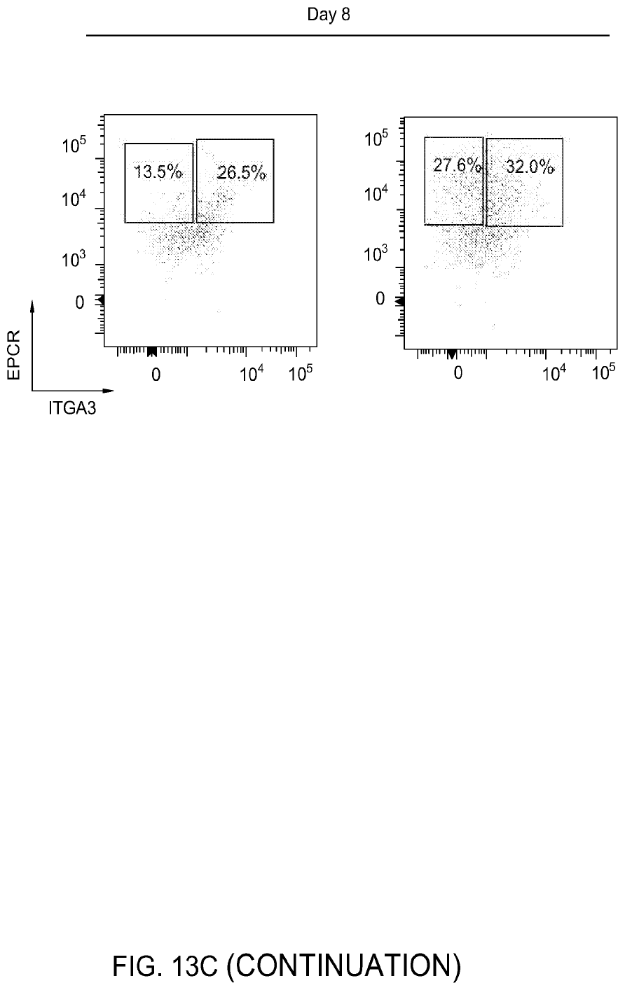

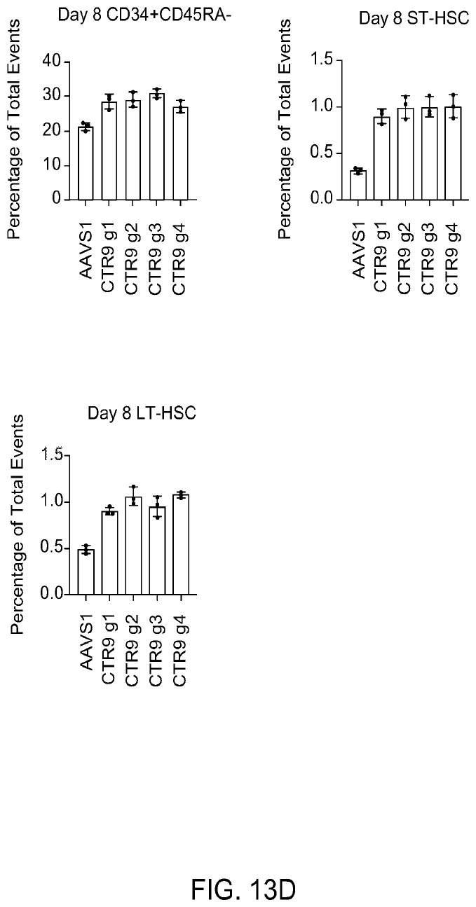

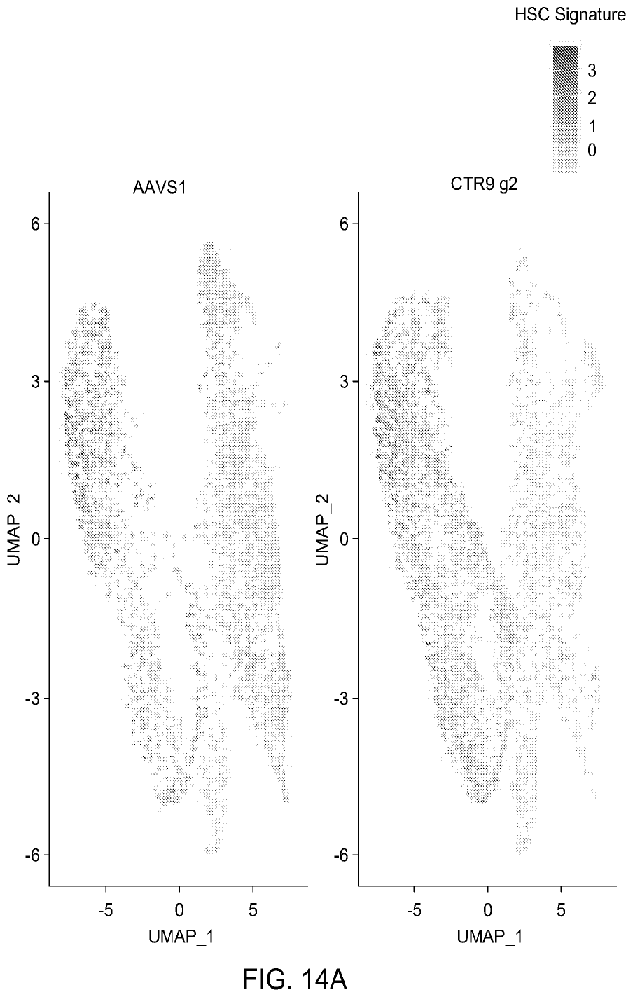

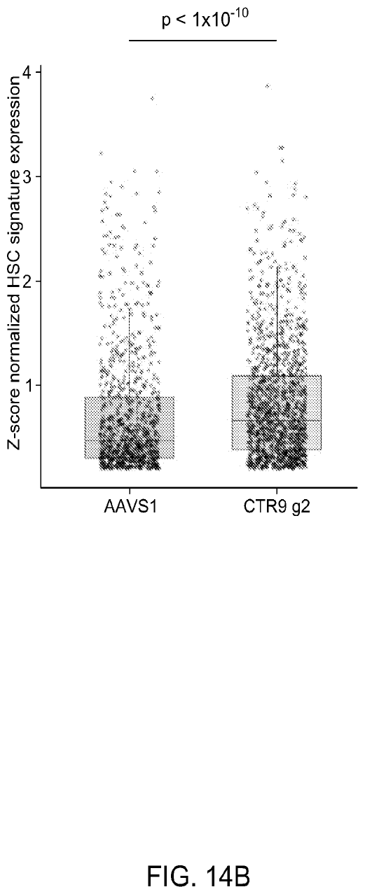

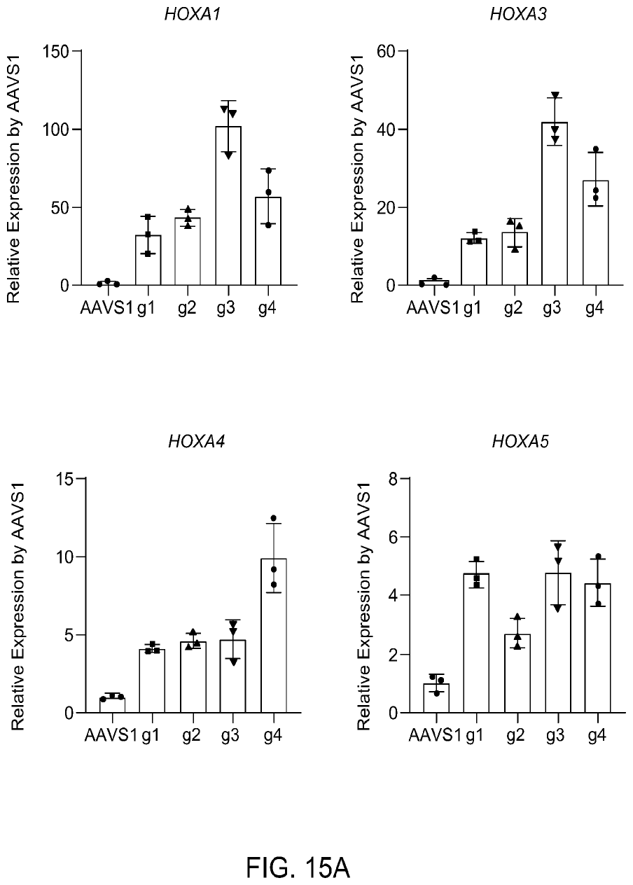

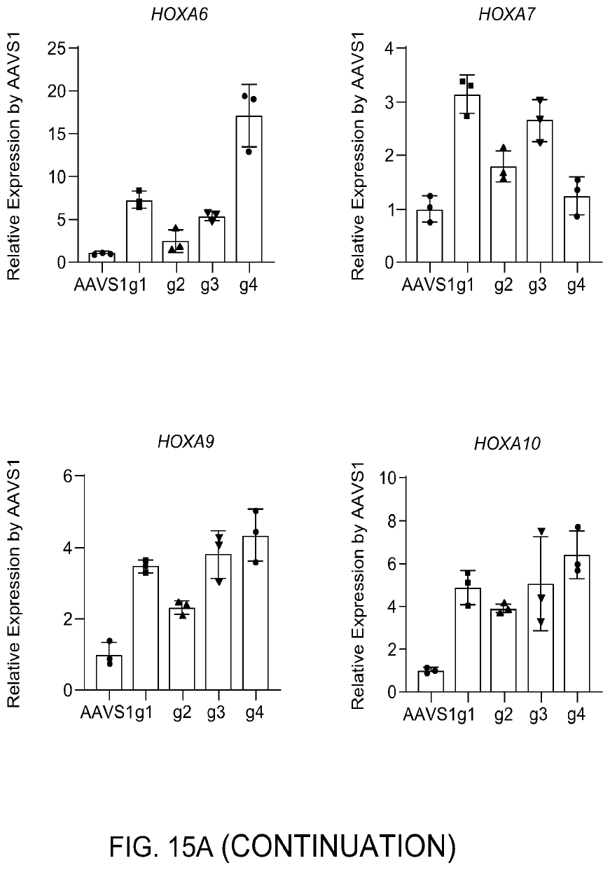

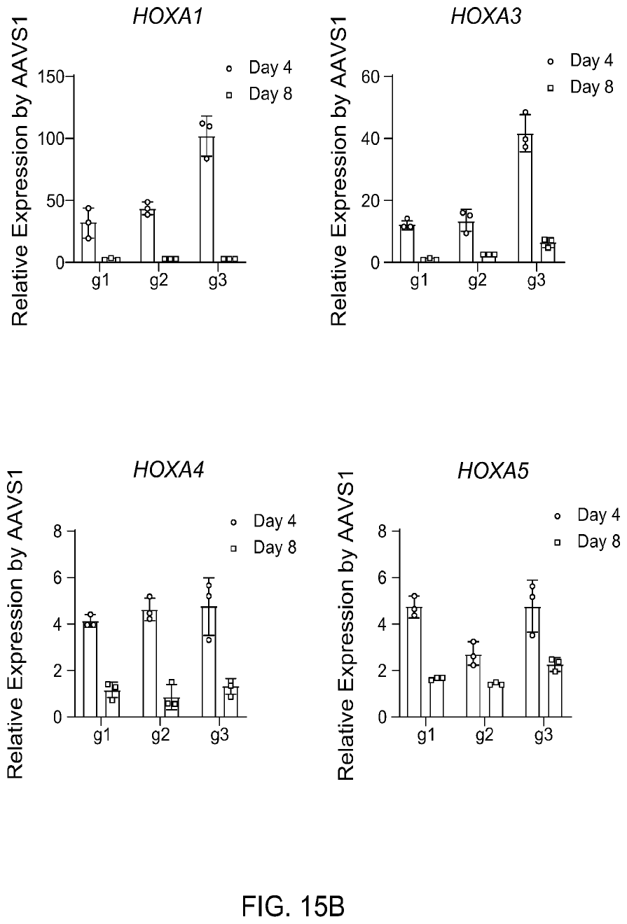

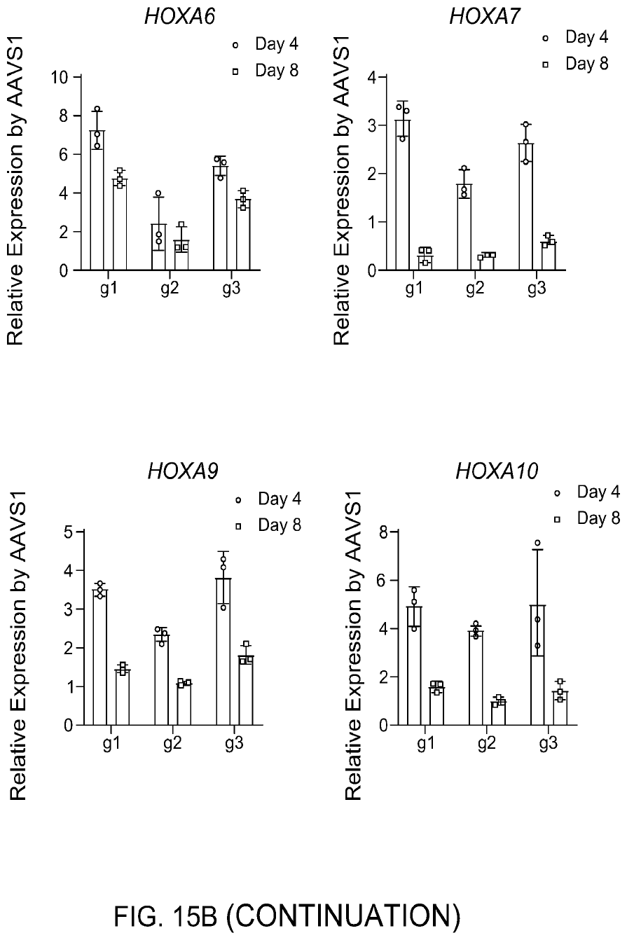

Increased hematopoietic stem cell (HSC) self-renewal has been shown to predispose to acquisition of myeloid malignancies (2, 17-19). Given the loss-of-function in CTR9 observed by risk alleles, it was hypothesized that these variants may impact HSC or progenitor self-renewal or function. To recreate loss-of-function alleles in an isogenic setting in primary human hematopoietic stem and progenitor cells (HSPCs), Cas9 ribonucleoprotein (RNP) delivery with four independent guide RNAs targeting CTR9 was used. Interestingly, there was an increase in phenotypic long-term repopulating HSCs (LT-HSC) (20) in the days after editing, but these cells were then depleted (FIGS. 11A-11D). It is noted that while CTR9 protein levels were partially reduced (˜50%) soon after RNP delivery, more profound depletion (>80%) occurred subsequently in tandem with the reduction in LT-HSCs (FIG. 11E). To more faithfully model heterozygous loss-of-function, the amount of Cas9 RNP delivered into HSPCs was titrated so that CTR9 would be edited in a predominantly heterozygous manner (FIGS. 12, 13A, and 13B). Strikingly, this editing achieved expansion of both the phenotypic LT-HSC and more differentiated short-term HSC (ST-HSC) compartments without any change in the overall number of HSPCs (marked by CD34⁺CD45RA⁻) across multiple time points in culture (FIGS. 3A, 3B, 13C, and 13D). As phenotypic markers can be limited, single-cell RNA sequencing (scRNA-seq) on 3,335 AAVS1 edited control and 4,154 CTR9 edited CD34⁺CD90⁺CD45RA⁻ cells was also performed and an expansion in cells harboring a previously defined HSC molecular signature was observed (2) with CTR9 editing (FIGS. 3C, 3D, 14A, and 14B). In addition to the observed phenotypic and molecular HSC expansion, an increased colony plating capacity of HSPCs with larger colonies noted upon CTR9 editing was also observed (FIGS. 3E and 3F). Given that prior studies have shown how HOXA family members are both necessary for HSC self-renewal and demonstrate increased expression with altered transcription elongation (21, 22), the major function of PAF1c and other interacting complexes, expression of HOXA family genes was examined and consistent increases accompanying the observed HSC expansion were seen (FIGS. 15A and 15B).

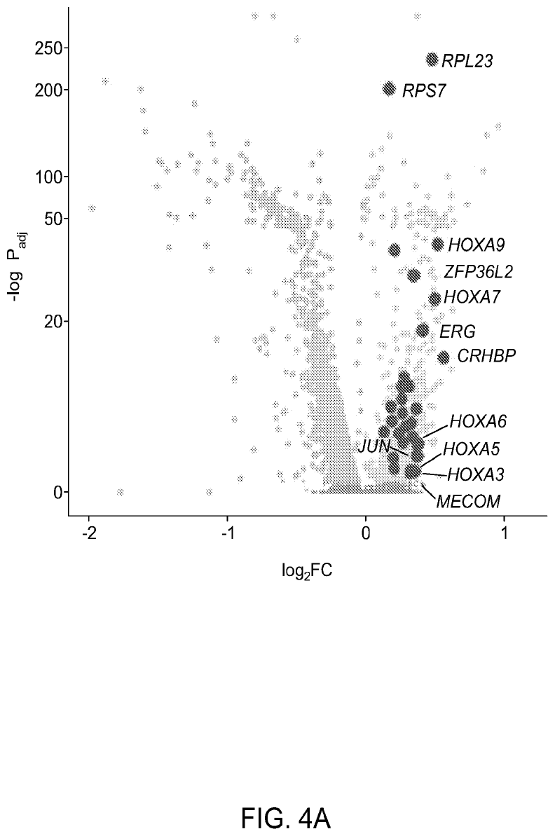

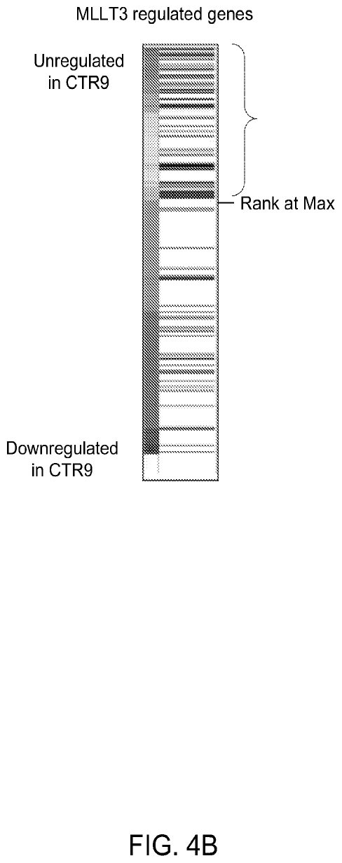

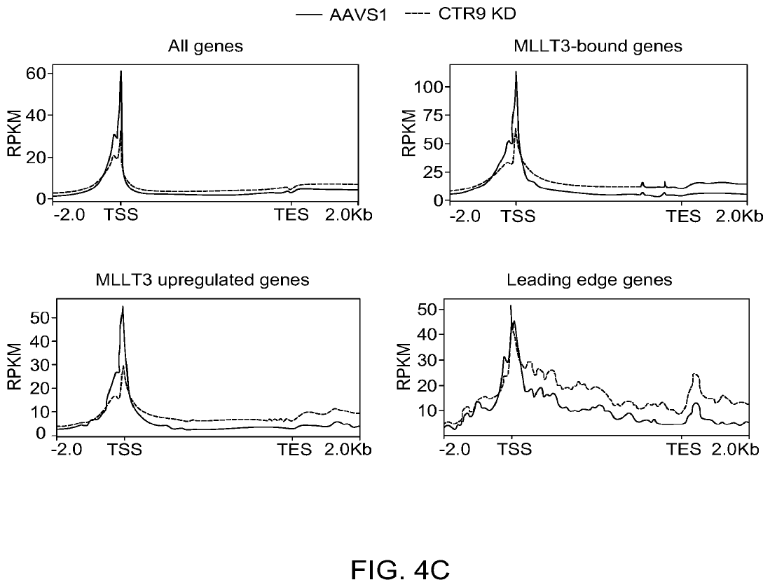

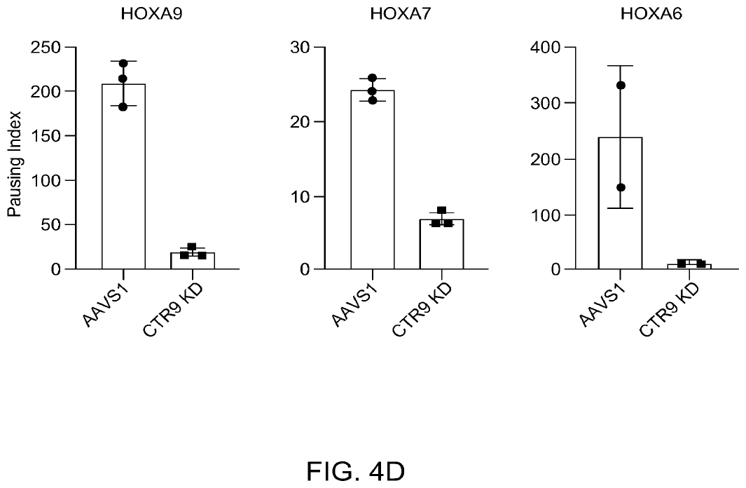

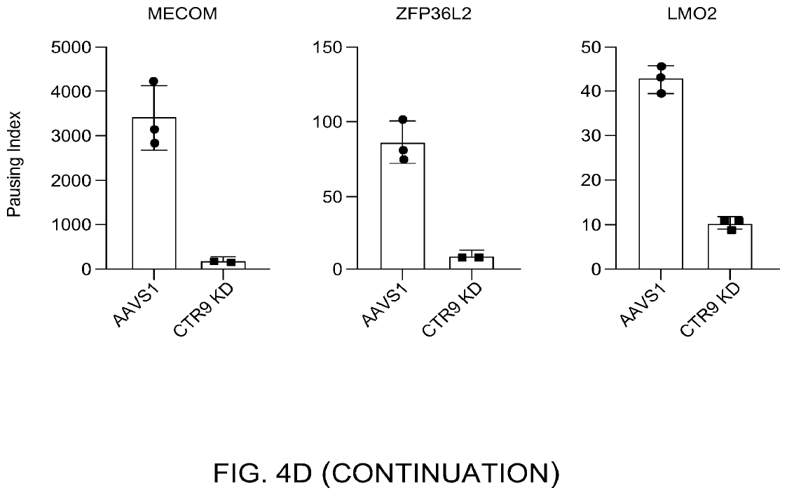

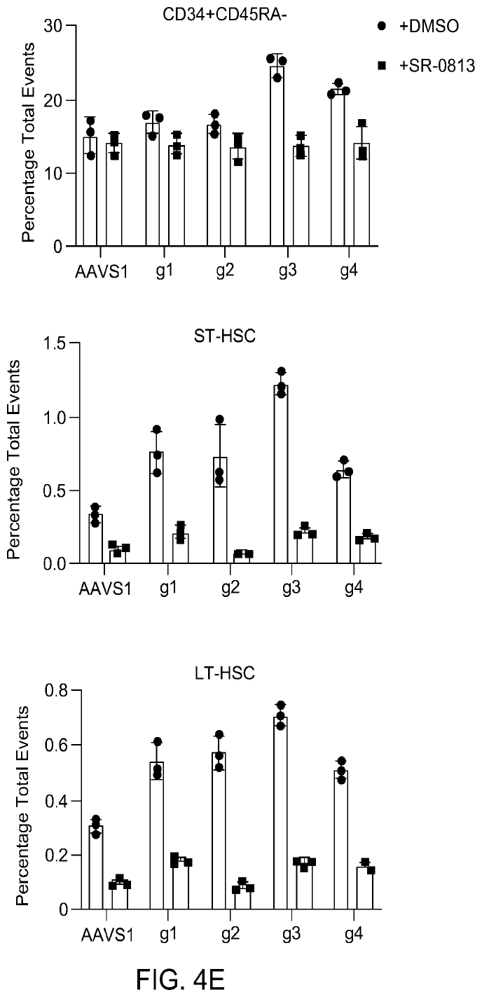

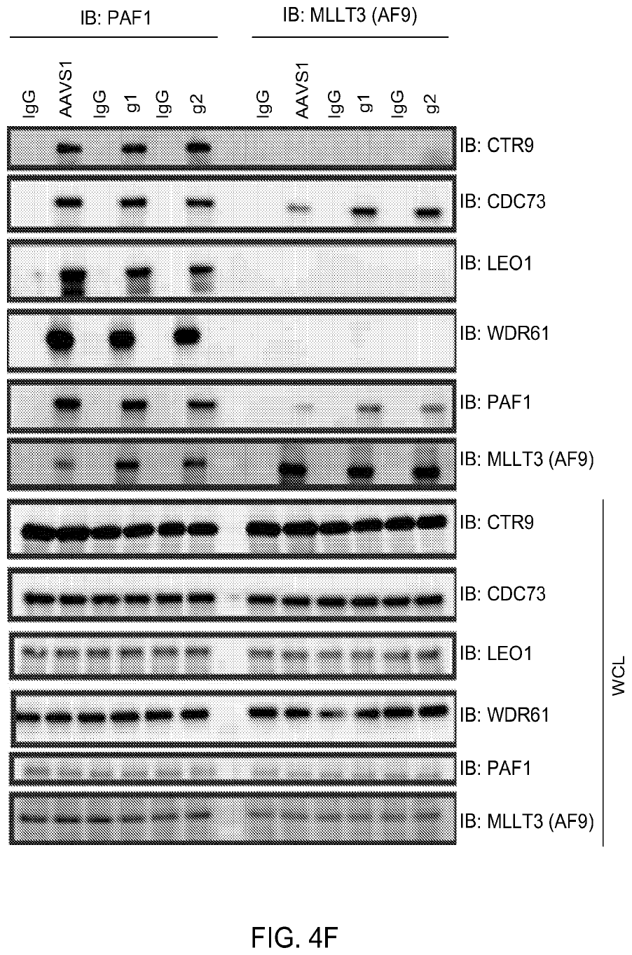

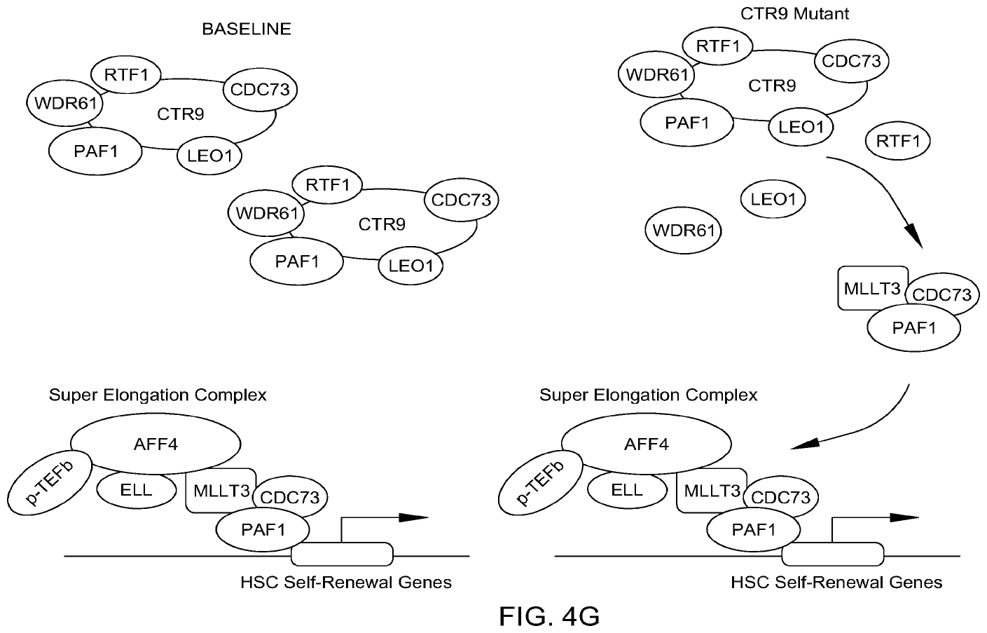

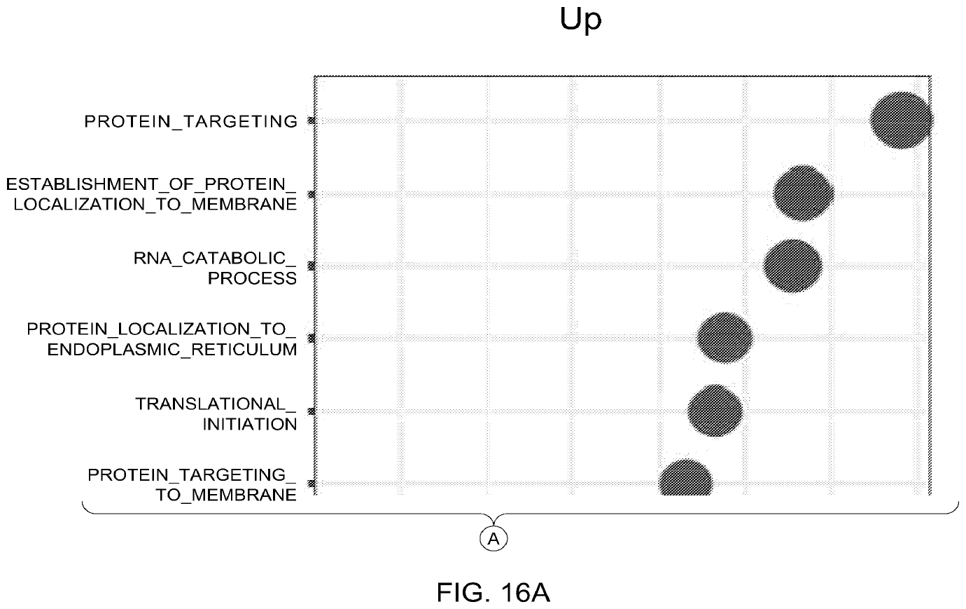

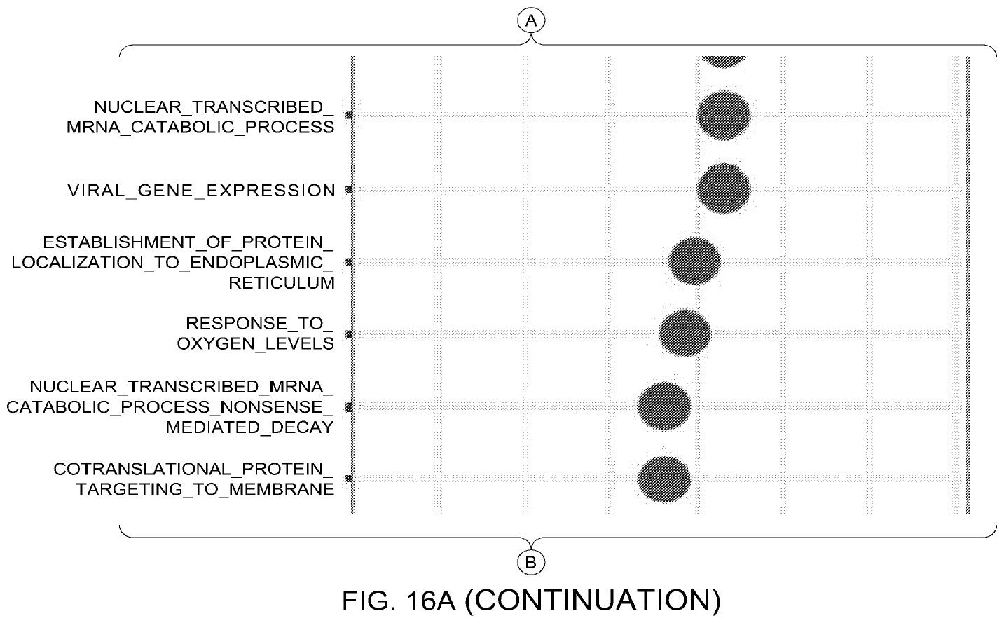

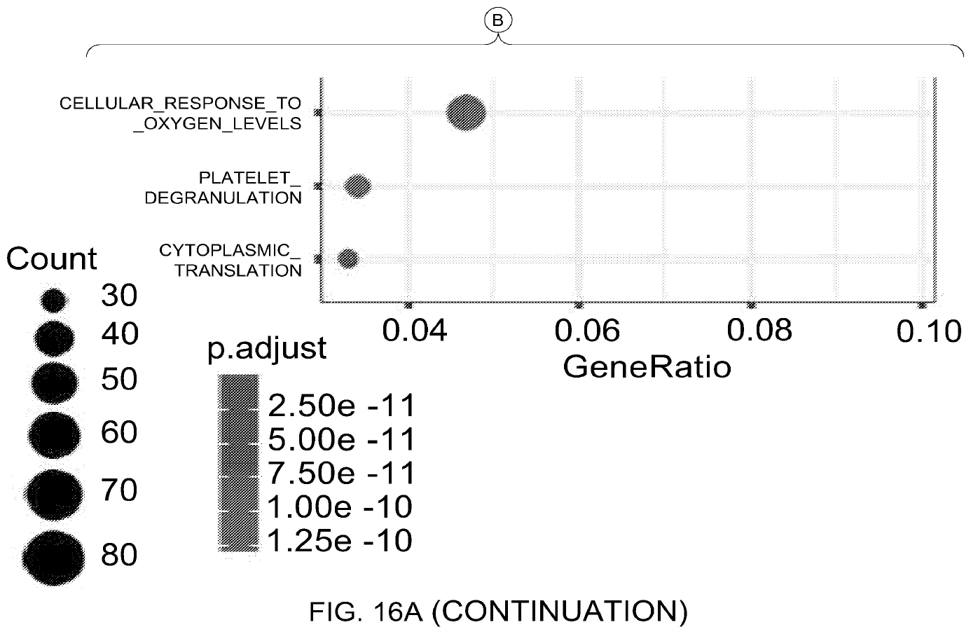

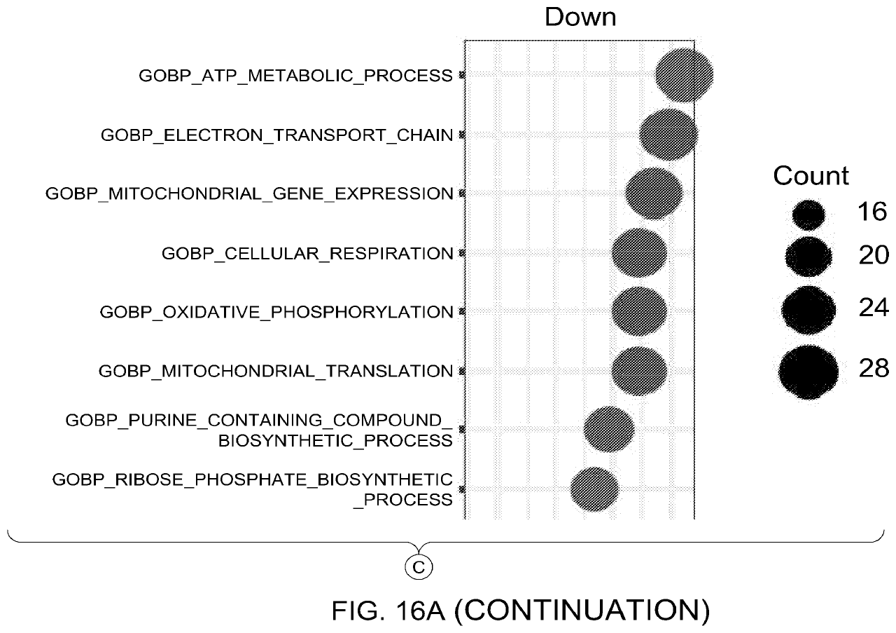

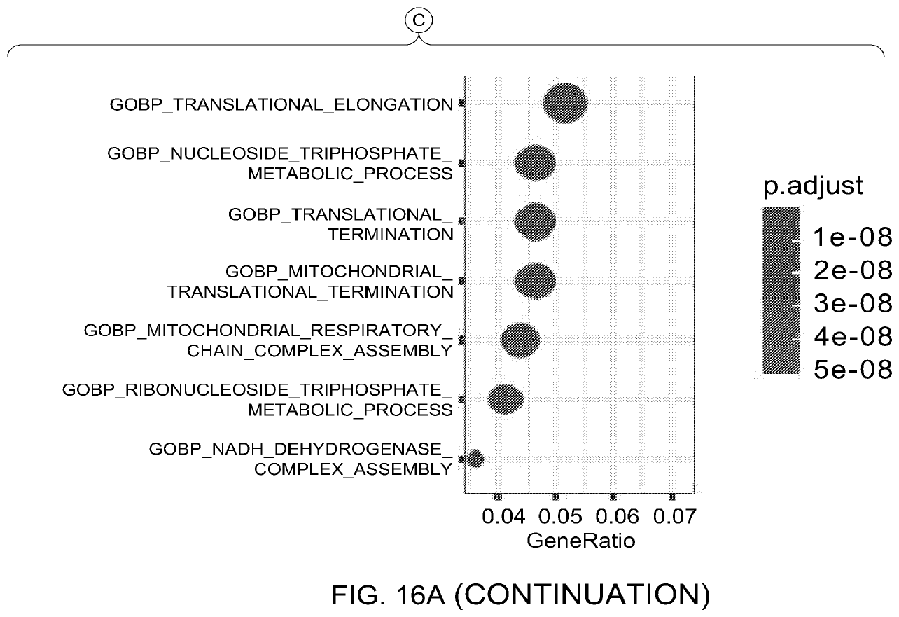

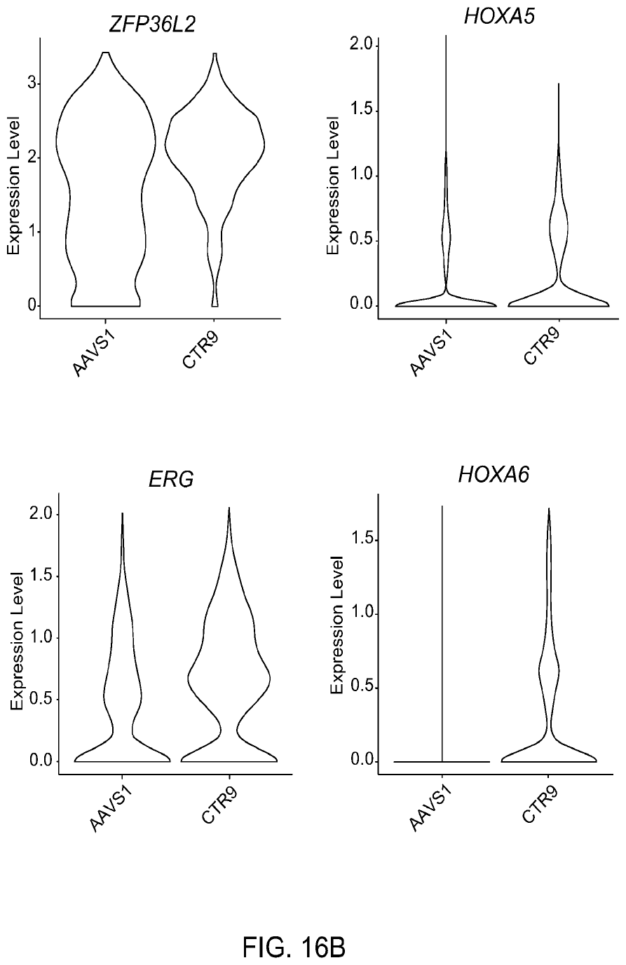

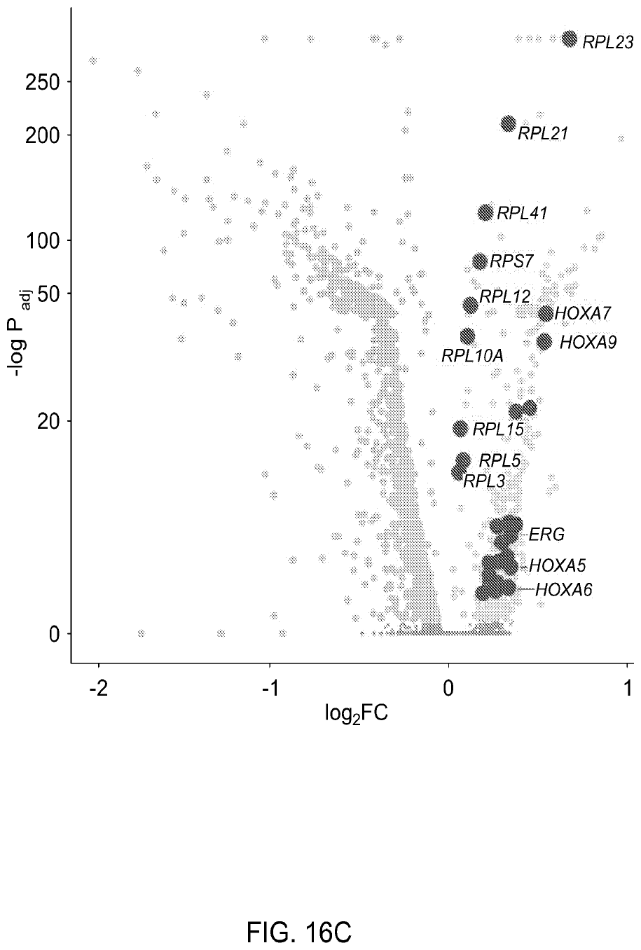

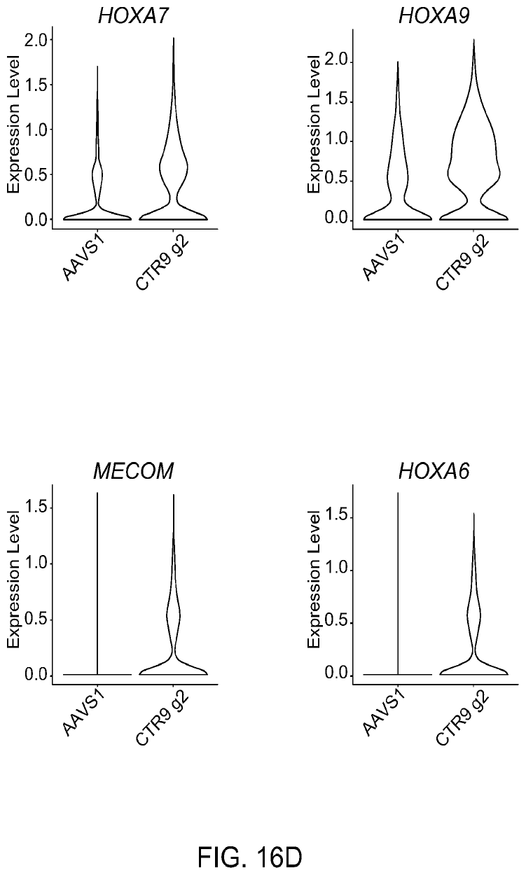

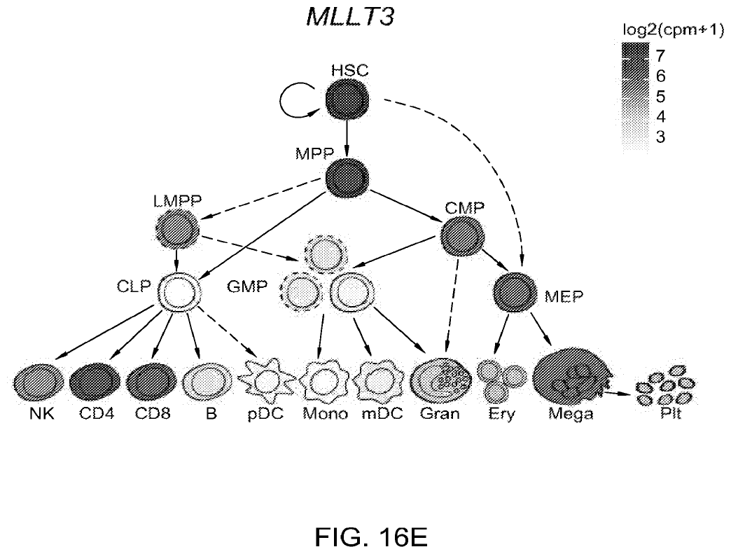

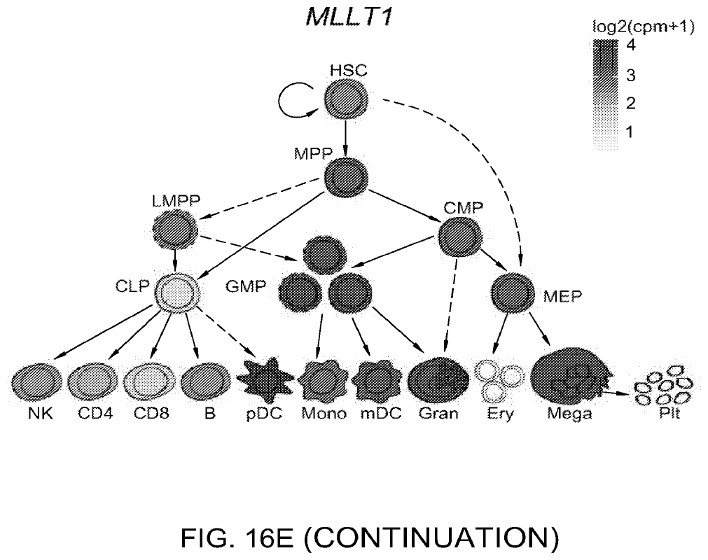

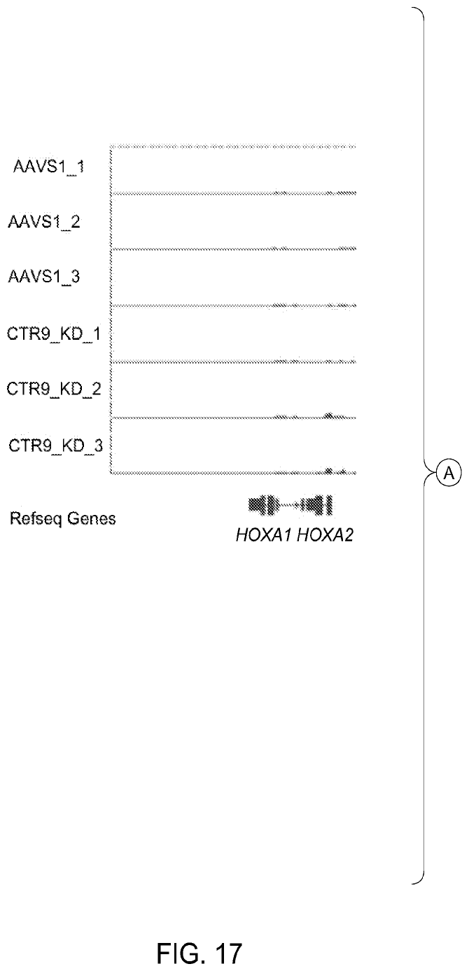

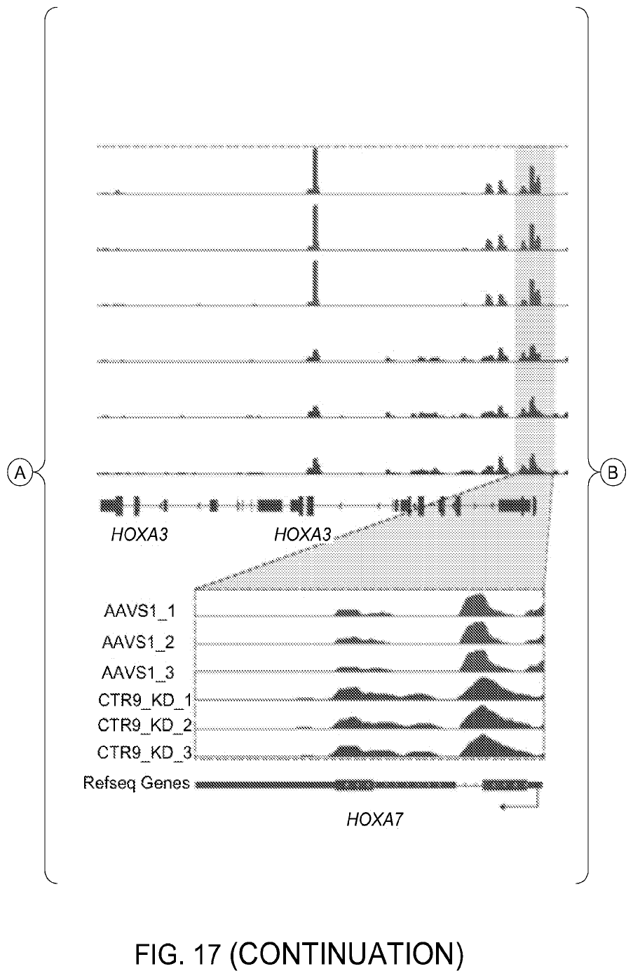

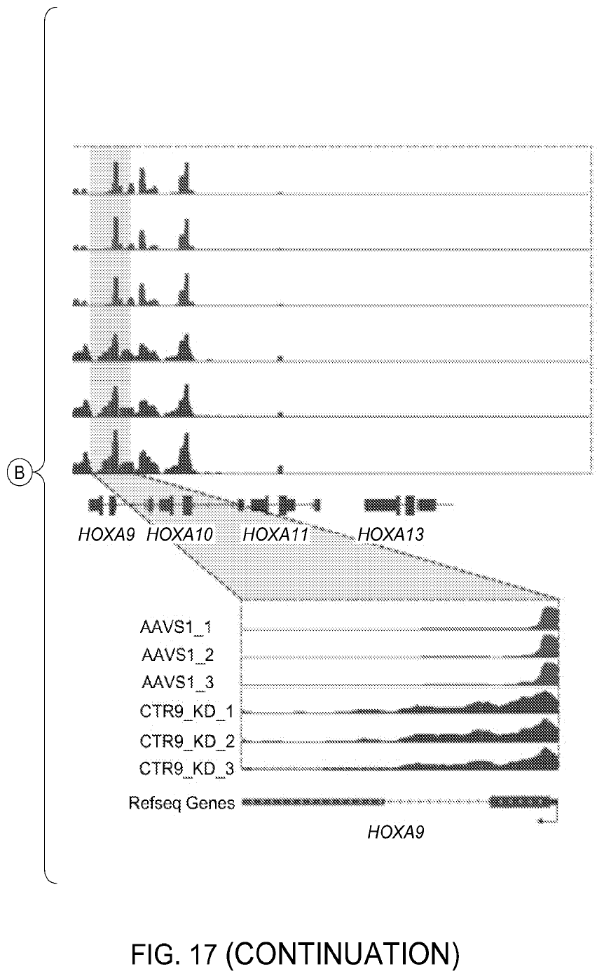

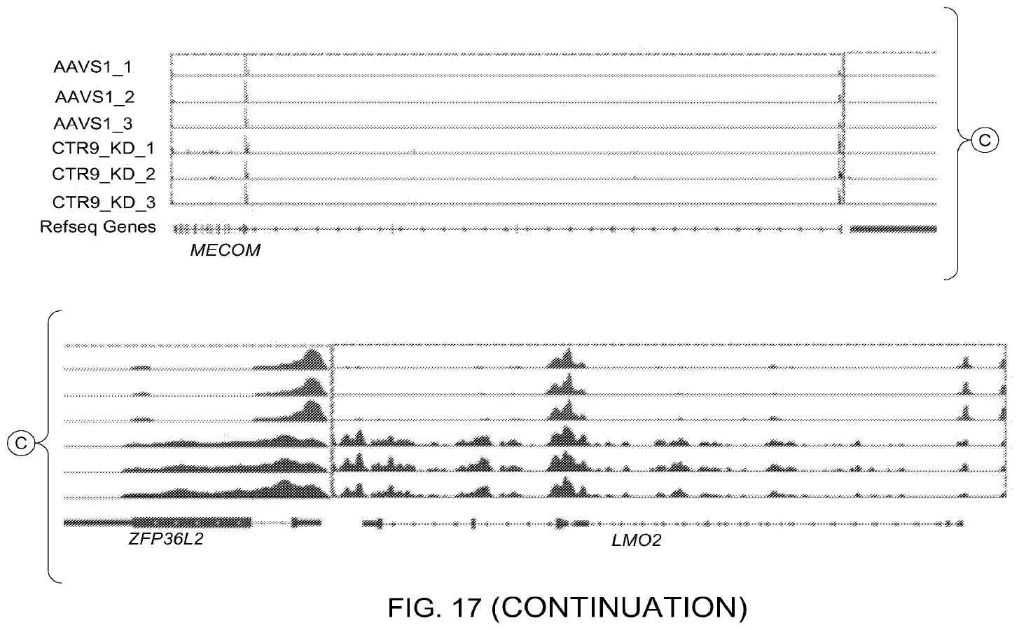

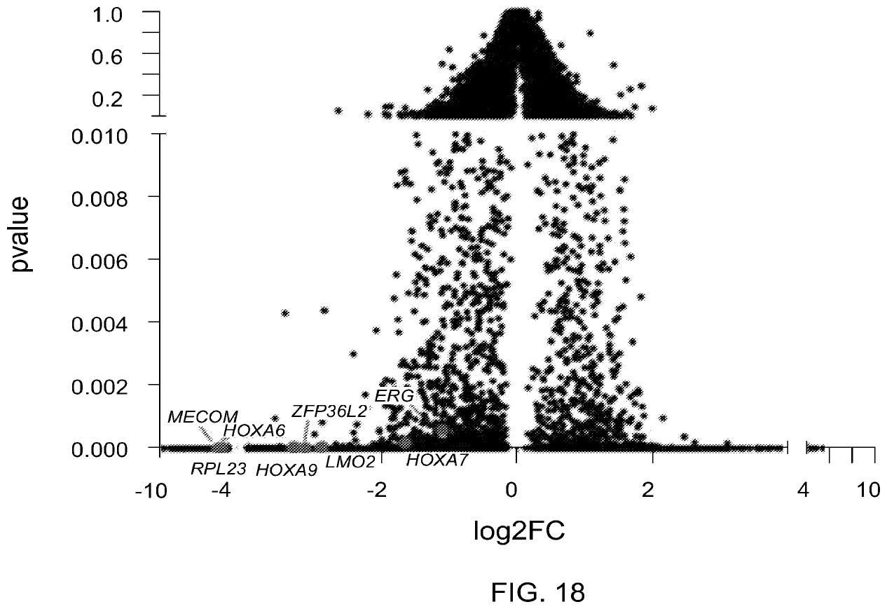

PAF1c is critical for maintenance of HSCs (23). Therefore, the observed HSC expansion and increased HOXA family gene expression with heterozygous loss-of-function of CTR9 appeared paradoxical and raised the question of underlying mechanisms by which blood cancer predisposition could arise. Differentially expressed genes in the HSC subpopulation were examined through scRNA-seq. While gene ontology analysis on the differentially expressed genes (both those that are either up or downregulated upon CTR9 editing), revealed a number of pathways including alterations in RNA processing and metabolism, protein translation, processing and membrane targeting, and cellular respiration (FIG. 16A), none of these provided clear insights into underlying mechanisms that could explain the observed HSC expansion. Given known interactions between PAF1c and components of the super elongation complex (SEC) (24-26), as well as the HSC expansion phenotype seen with increased expression of SEC component MLLT3 (also known as AF9) (27), which is similar to what were observed with CTR9 heterozygous loss-of-function, the extent to which targets of MLLT3 in human HSCs may be upregulated upon CTR9 editing was examined. Remarkably, there was a significant enrichment of MLLT3 upregulated genes that were increased upon CTR9 editing in HSCs by gene set enrichment analysis (normalized enrichment score=3.29, p-value <0.001, FIGS. 4A and 4B). These targets included many key HSC self-renewal regulators including mid to posterior HOXA genes and MECOM, and similar findings were also observed with an independent guide RNA (FIGS. 16B-16D). Since increased SEC activity should promote transcription elongation, nascent RNAs were examined using precision run-on sequencing (PRO-seq) in edited CD34⁺HSPCs (Methods). While there was a slight global increase in transcription elongation, the major contributions appeared to come from known MLLT3 targets in human hematopoiesis and from genes that are upregulated in the scRNA-seq data from HSCs (FIGS. 4C and 17). Indeed, these genes showed dramatic downregulation in their calculated pausing indices, indicating that increased transcription elongation underlies their increased expression (FIGS. 4D and 18).

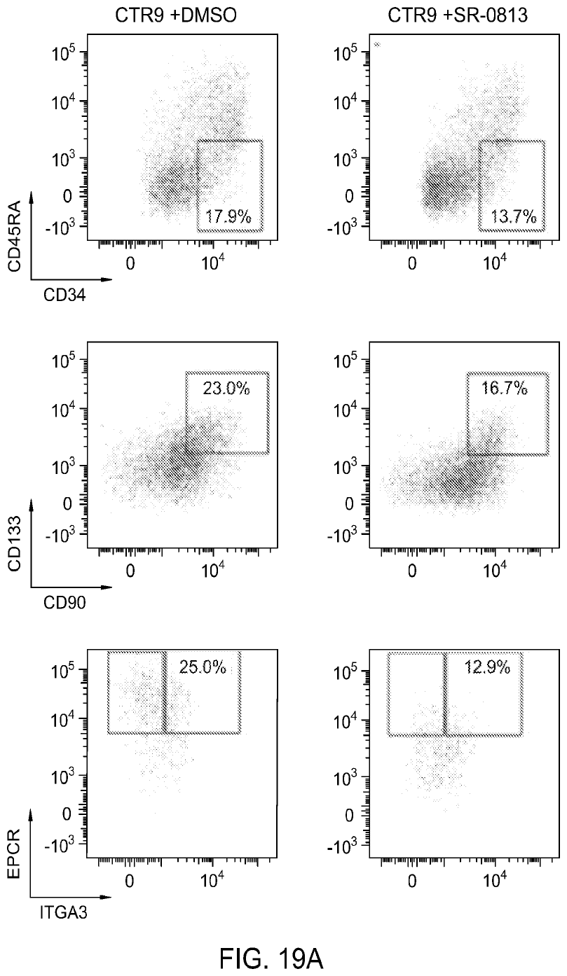

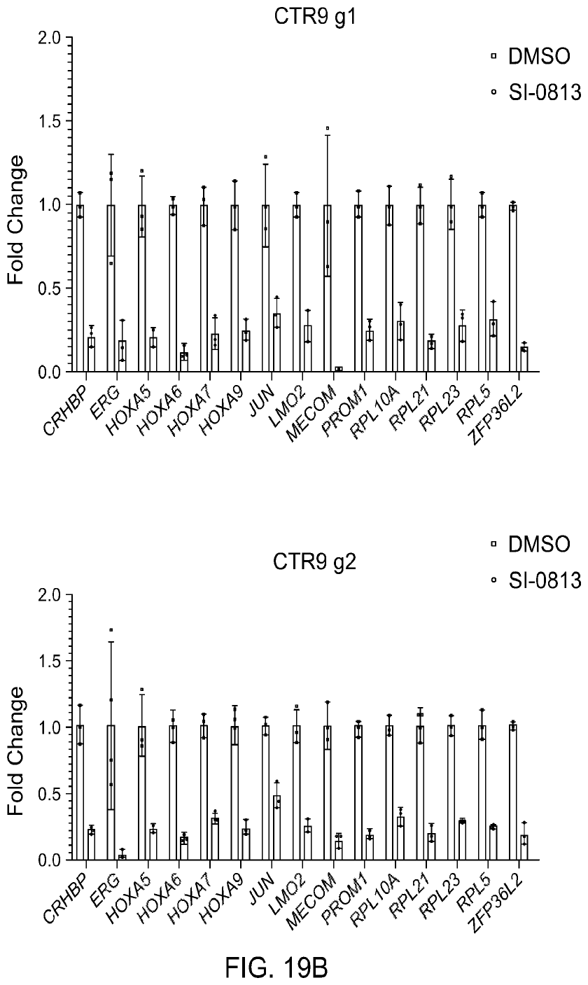

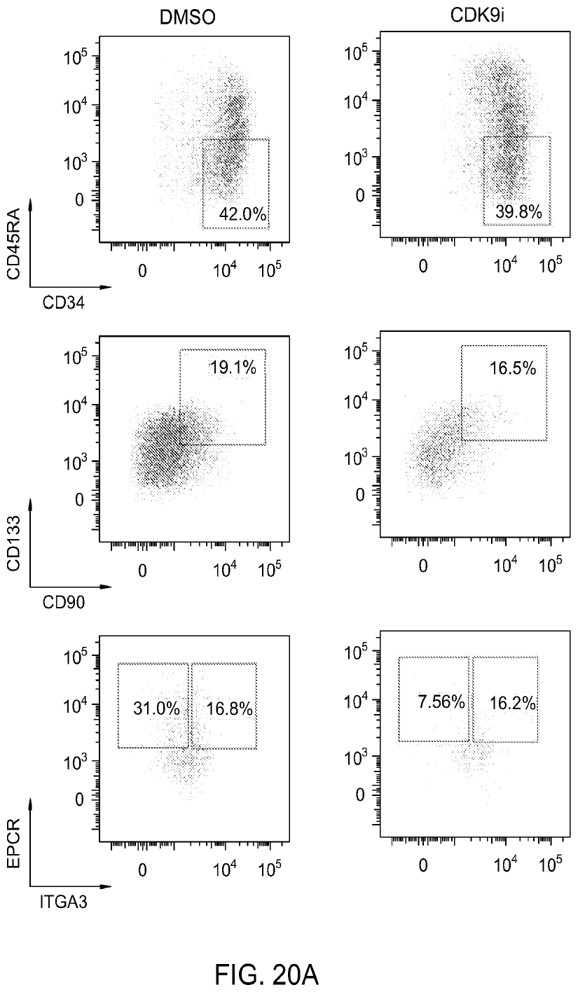

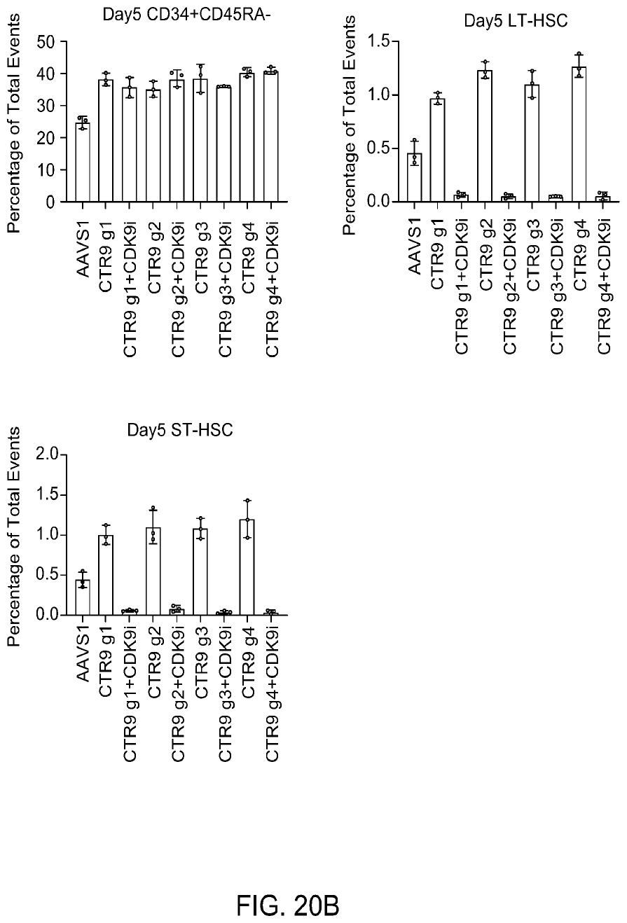

In light of these compelling findings indicating that SEC activity may be increased with CTR9 heterozygous loss-of-function, it was critical to functionally validate these observations. A recently described specific and potent inhibitor of the YEATS domains of MLLT3 and MLLT1 (also known as ENL) (28-30) was used. While one cannot formally discriminate between targeting of MLLT3 vs. MLLT1, MLLT3 is much more highly expressed in human HSCs than MLLT1 (FIG. 16E), suggesting that MLLT3 is the major target in primary human HSCs. Using the YEATS domain inhibitor, SR-0813, it was noted that suppression of phenotypic LT- and ST-HSC expansion occurred without a major impact on the bulk HSPC population. Importantly, however, this inhibitor completely suppressed the CTR9-perturbation-mediated HSC expansion and concomitantly downregulated many of the key upregulated target genes (FIGS. 4E, 19A, and 19B). To ensure that this suppression of MLLT3 was acting through the SEC, CDK9 was also inhibited, a key kinase subunit of the p-TEFb subcomplex in the SEC that phosphorylates RNA polymerase II to promote transcription elongation, and observed a similar rescue of HSC expansion by CTR9 loss (FIGS. 20A and 20B).

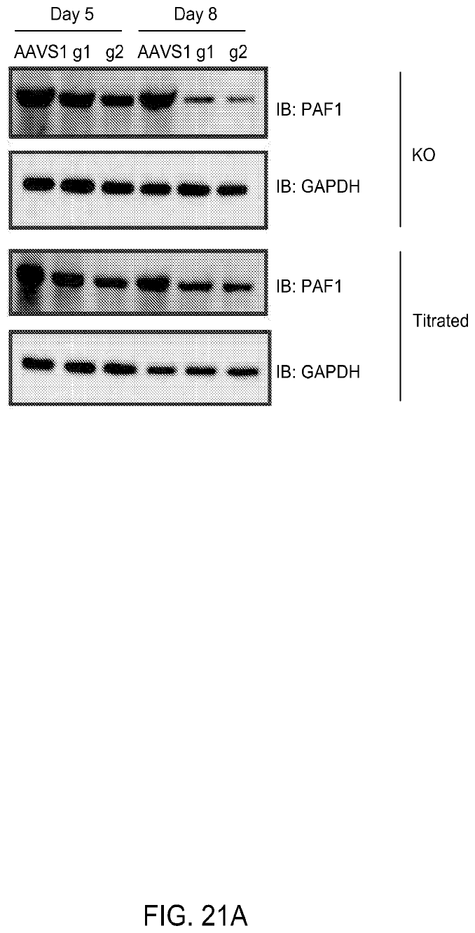

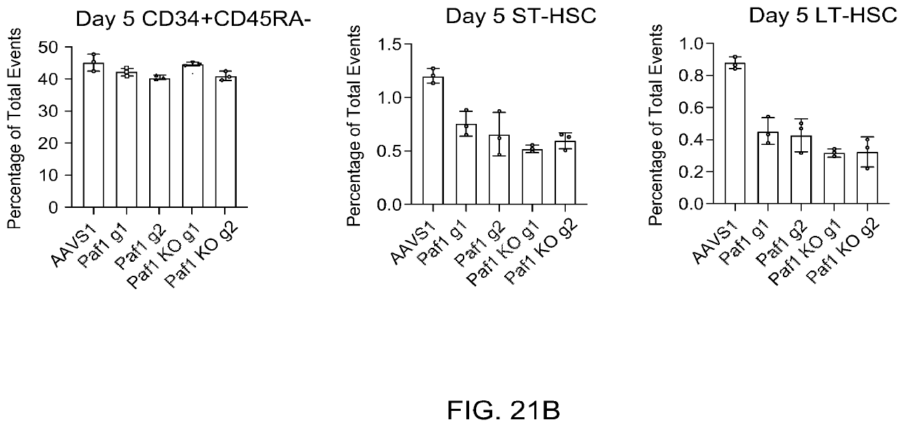

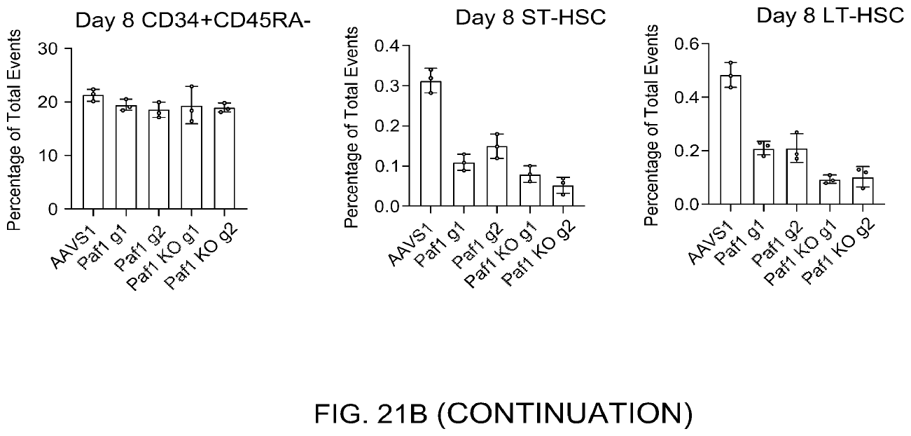

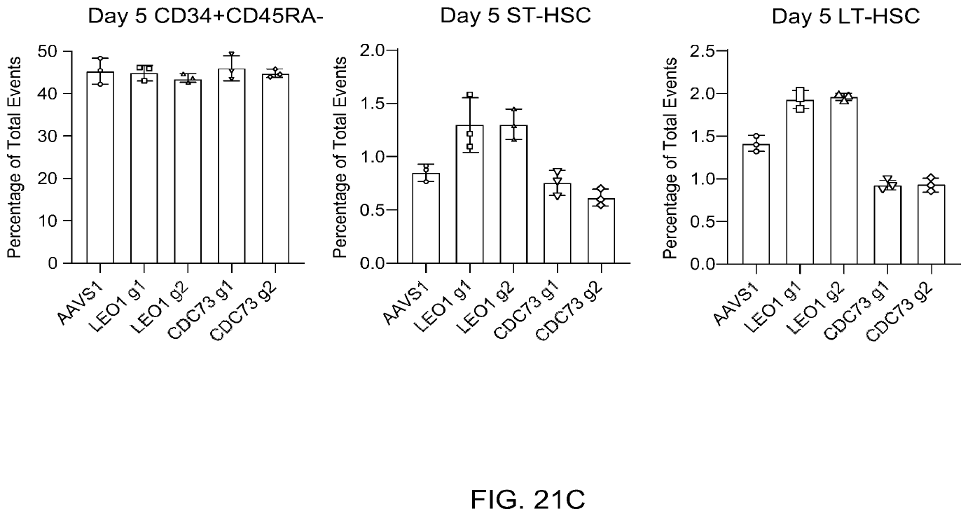

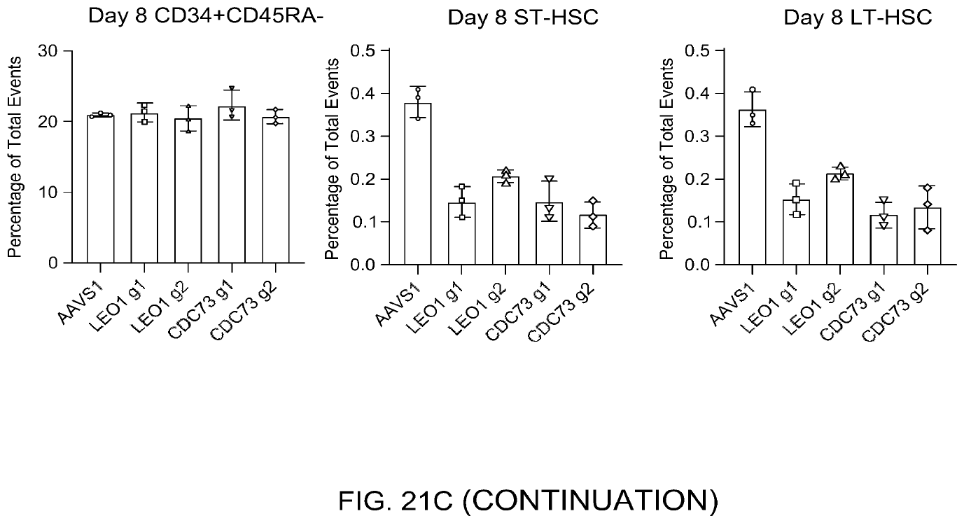

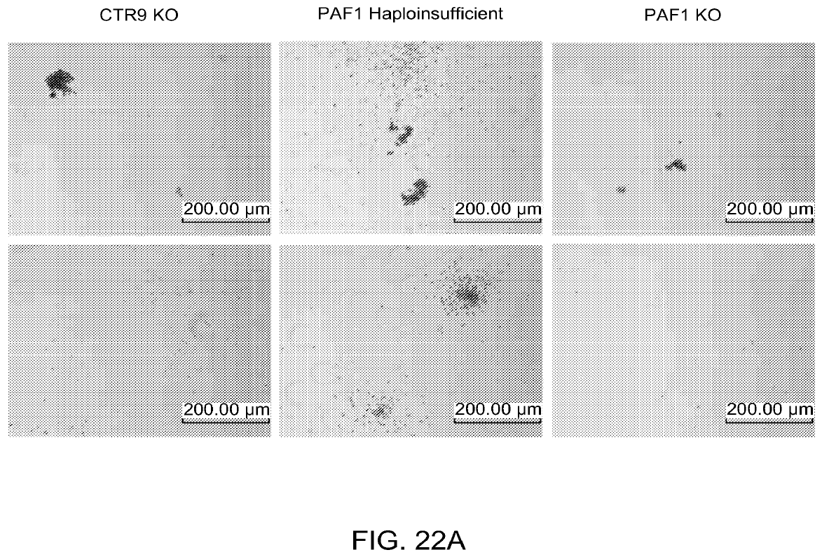

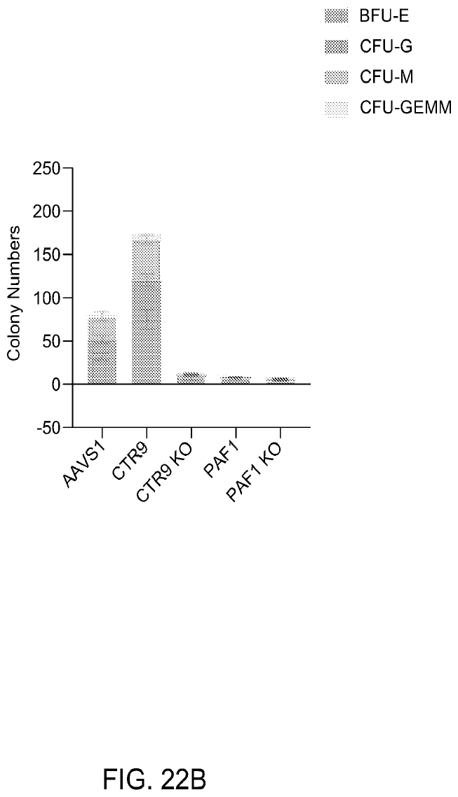

Given the previously reported antagonism between PAF1c and the SEC (24), as well as given the specific alterations and myeloid malignancy predisposition observed due to CTR9 loss, the possibility that partial loss of CTR9 results in altered PAF1c complex assembly and this thereby results in the increase in SEC activity observed was investigated using primary human HSPCs. When PAF1 was pulled down, all binding partners from PAF1c were co-immunoprecipitated as expected, but co-immunoprecipitation of MLLT3 was also observed. Importantly, the amount of MLLT3 interacting with PAF1 increases when CTR9 is partially lost, suggesting that CTR9 might act as an inhibitor of this interaction (FIG. 4F). In contrast, when MLLT3 was immunoprecipitated, only the PAF1 and CDC73 subunits of the PAF1c co-immunoprecipitated with MLLT3, and the amount of co-immunoprecipitation also increased with CTR9 editing (FIG. 4F). Therefore, these results indicate that the altered HSC self-renewal is due to promotion of increased SEC activity in the setting of reorganized binding by specific PAF1c subunits, including PAF1 and CDC73. To functionally assess the impact of perturbing these other PAF1c components on human HSCs, PAF1 and CDC73 were edited. In contrast to CTR9, the phenotypic HSC populations were depleted even at the early time point in culture (FIGS. 21B and 21C). Moreover, even Cas9 RNP titration to achieve heterozygous PAF1 editing resulted in depletion of HSCs and progenitors (FIGS. 21A, 21B, 22A, and 22B). Interestingly, when another subunit of PAF1c that does not interact with MLLT3, LEO1 was edited, an expansion of phenotypic HSCs was observed at an early time point, as was the case with CTR9 (FIG. 21C). These functional experiments in tandem with the biochemical studies validate a model whereby heterozygous loss of CTR9, which confers significant predisposition to myeloid malignancies, frees specific PAF Ic subunits that can then cooperate with components of the SEC, including MLLT3, to enable productive transcription elongation of genes necessary for HSC self-renewal (FIG. 4G).

Through rare variant association studies, CTR9 was identified as a risk gene harboring deleterious variants that confer significant predisposition to myeloid malignancies. Beyond expanding an understanding of blood cancer risk, the data presented herein also gain insights into how altered transcription elongation can increase HSC self-renewal and thereby confer such risk. Changes in the PAF1c that activate the SEC in hematopoietic cells were defined and previously unappreciated biochemical and functional interactions mediating these activities were characterize. Experiments and work described herein focus on changes that precede acquisition of somatic driver mutations, since ˜0.1% of individuals carry a distinct deleterious mutation in CTR9 conferring a ˜10-fold odds of acquiring a myeloid malignancy. This is far more common than the prevalence of individuals diagnosed with myeloid malignancies. Nonetheless, there may be roles for cooperativity between these germline variants and somatic driver mutations that should be investigated in the future. Importantly, beyond identification of a cancer predisposition gene, these findings also pave the way for potential cancer prevention strategies through the modulation of transcription elongation and suppression of HSC self-renewal in individuals at high risk for acquiring blood cancers.

### Materials and Methods

### UKB RVAS

UK Biobank (UKB) Whole Exome Sequence (WES) data (Category 170, UKB Showcase) on 200K individuals was accessed under application 31063. Written consent is in place for all UKB participants, any participants whom have since withdrawn their consent from the UKB were removed from this analysis. Exome capture was conducted using IDT xGen Exome Research Panel v1.0 including supplemental probes. The population-level Variant Call Format (pVCF) file was created by UKB as described elsewhere (31) (methods sections: OQFE protocol and OQFE mapping and variant calling). All data is aligned to the GRCh38 reference genome.

### Quality Control

The original, unfiltered, pVCF from UKB was subjected to several filtering requirements prior to analysis. Filtering was performed in Hail (32). Genotypes: Genotype entries were hard filtered using DP>10 and GQ>20. Allele balance was required to be within 0.25 and 0.75 if a heterozygous call, or <0.1, >0.9 if a homozygous call. Samples: Samples were excluded if they were not of self-reported White-British ancestry with the same genetic ancestry inference based on a principal components analysis, not included in kinship inference, an excess (>10) of putative third-degree relatives inferred from kinship, an outlier in heterozygosity and missing rates, and putative sex chromosome aneuploidy (generated by UKB). Samples were also filtered out if they had a missing rate of >0.1. Variants: Variants were excluded if they were multiallelic, had a missing rate >0.1, hardy-weinberg equilibrium p-value of >1e-15. Variants were required to have an allele frequency of <0.01 (1%) to be classified as rare, anything above this threshold was filtered.

Annotation and determination of deleterious variants. Variants in the filtered pVCF files were annotated using VEP (33) (including the LOFTEE (34) plugin), ANNOVAR (35) (using dbNSFP v4.1 (36), ClinVar v20200316 (37) and ALoFT (38)), and InMeRF (39). Genes were annotated using RefSeq (via ANNOVAR). Loss-of-function (LoF) was determined by LOFTEE and ALoFT tools, variants were required to be high-confidence (HC), with none of the following warning flags (“SINGLE_EXON”, “NAGNAG_SITE”, “PHYLOCSF_WEAK”, “PHYLOCSF_UNLIKELY_ORF”, “NON_CAN_SPLICE”). Missense variants were determined by an InMeRF score of >0.5. The choice of InMeRF to determine deleterious variants was made from an AUROC performance analysis of several existing tools in dbNSFP. Classifying variants (True Positives) used were ClinVar (September-2018 release) “Pathogenic” clinical significance interpretation (CLNSIG) filtered to only include high confidence variants using the CLNREVSTAT field: “multiple submitters”, a “reviewed by expert panel” or using “practice guideline”, and “no conflicts” (N=4460). True negatives were common benign missense variants were obtained from the gnomAD database (in this database there are no known severe Mendelian disorders and as such it is assumed that highly penetrant disease-causing missense variants will be rare in this database (allele frequency <1%)), these were downsampled for computational efficiency (N=9200). InMeRF provided the highest performance (AUROC=0.83), significantly higher than the second highest AUROC (MetaSVM), p=0.007. Importantly, multiple compound combinations of tools were also estimated to have lower performance than InMeRF classification alone (Data not shown).

### Phenotype Derivation

To gain the largest degree of power and because of the view that these phenotypes share many elements, myeloproliferative neoplasm (MPN), acute myeloid leukemia (AML) and myelodysplastic syndrome (MDS) were combined into a Myeloid Malignancy mega-phenotype for primary analysis. The individual phenotypes were curated as follows. MPN: polycythemia (ICD10 D45; ICD9 2384), essential thrombocythemia (ICD10 D47.3, D75.2), osteomyelofibrosis (ICD10 D47.4, D75.81), chronic myeloid leukemia (ICD10 C921, C922, C931; ICD9 2051), and chronic myeloproliferative disease (ICD10 D47.1). AML: acute myeloid leukemia (ICD10 C92.0, C92.4, C92.5, C92.6, C92.8; ICD9 2050). MDS: myelodysplastic syndrome (ICD10 D46.0, D46.1, D46.2, D46.3, D46.4, D46.5, D46.6, D46.7, D46.9). Individuals were also classified as cases if they had a self-reported cancer, self-reported illness code, or histology of cancer tumor code for any of the above.

### Association Testing

When collapsing, only genes for which there was a combined minor allele count (cMAC) of 20 or greater were included in the association analysis. Only deleterious variants were used in the collapsing association test, i.e. classified LoF or disruptive missense. Association was tested using SAIGE-GENE v0.44.1 (40), which utilizes a robust saddle point approximation (SPA) SKAT-O approach to collapsing tests in a generalized linear mixed model. The genetic relationship matrix (GRM) for step 0 and 1 was formed using common markers (allele frequency >0.01) and was formed using PLINK (41, 42). The following were included as covariates in the association testing: age, gender, first 10 principal components of ancestry. In addition, the originally sequenced 50K exome samples used a different IDT v1.0 oligo lot so a covariate was included to label this subset (43). To correct for the multiple genes across the genome being tested, p-values were adjusted using the FDR Benjamini-Hochberg procedure, any gene unit with a q-value less than 0.05 (5% FDR) was considered significant.

### Plotting

Results were composed into a Manhattan style plot, by obtaining the RefSeq start coordinates of the genes. Plotting was done in R v4.0.3 (44), using ggplot2 (45).

### Estimation of Effect Size

Effect size was estimated in a cohort of unrelated samples. The KING kinship coefficient was calculated and used to determine those in 3rd-degree relation or greater, then one of the sample pairs was removed. Priority was given to cases, afterwards exclusion was based on random selection. To allow convergence despite rare events, three methods were used to determine effect size for significant genes, penalized maximum likelihood firth-biased logistic regression (using brglm2 (46)), bayesian MCMC logistic regression (using rstanarm (47)), and generalized logistic regression (using glm-base).

### CTR9 Germline Assessment

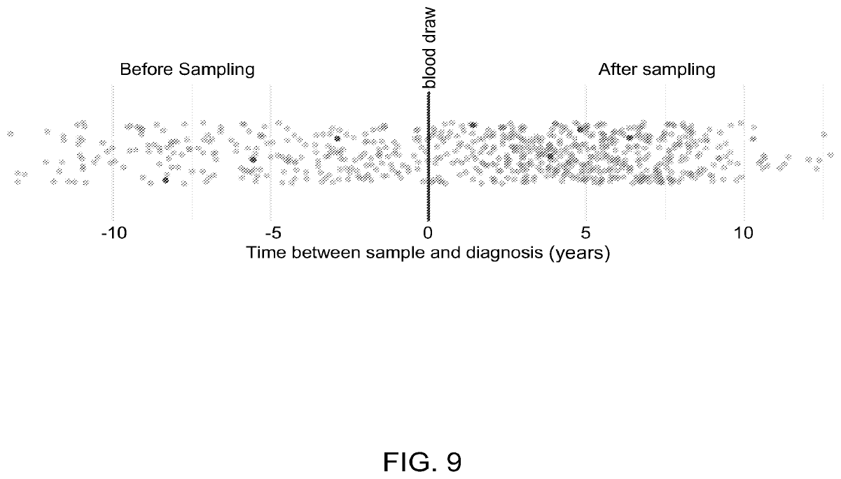

Several ancillary approaches to most robustly classify deleterious CTR9 variants as likely to be germline were pursued. First, as is becoming more common in WGS/WES sequencing analyses, VAF or percentage alternative reads (48) were analysed and found CTR9 variants consistent had a VAF over 0.3 (49) (FIG. 5). Although helpful, VAF does not completely enable separation between somatic clones with large allelic burdens >0.3/0.4, particularly for 3 samples obtained from the blood. Second, age at blood sampling compared to diagnosis was analysed. It is known that somatic clone size is often largest around diagnosis, somatic clones in MPN are detectable, in some cases, years before diagnosis, but are often of small VAF. Years after diagnosis, cytoreductive therapy often reduces clone size and VAF (50). It was found that in 4 of the CTR9 deleterious variant carriers, blood was sampled years before diagnosis, and that in the remaining 3 blood was sampled at least 5 years after diagnosis (FIG. 9). Both scenarios would suggest a smaller clone size than the average VAF detected in CTR9 at ˜0.45. In addition, none of the deleterious CTR9 carriers progressed to AML which would be the most likely of the myeloid diseases here to possess a clone likely to progress to a large VAF. Indeed, it is known that CTR9 does not harbor driver mutations in CHIP or myeloid malignancies. Through analysis of COSMIC, a widely used somatic mutation database with just over 336,000 hematopoietic cancer tissue samples, no evidence for CTR9 variants acting as driver mutations in hematologic malignancies were observed (8). Of those 336,000 only 4 carry confirmed somatic variants in the CTR9 gene making the probability (4/336,000=0.00001) of somatic mutation in CTR9 very small. Lastly, it is noted in a comprehensive recent review on this topic from Kraft and Godley, (49) that “diagnosis of a hematopoietic malignancy at a younger age than seen in the general population” is a factor leaning towards germline variation. In the analysis presented herein, it is found that CTR9 deleterious variant carriers have a median age at diagnosis of 59 [50-62] years old whereas other myeloid malignancy cases that are not carrying deleterious CTR9 variants are on average 64 [57-70] years old, p=0.10 (Wilcoxon rank sum). Although this is a nonsignificant difference, there is certainly a tendency towards younger age in the carriers, and if there were more carriers of these rare deleterious variants in CTR9 then a significant difference in age would be expected. Somatic variants using Mutect2 were also called, and found no evidence of somatic calls in the CTR9 gene. Familial cases are not present to analyze. All of this taken together indicate that the observed deleterious alleles in CTR9 are likely to be in the germline.

### Replication of CTR9

A cohort of 211 MPN patients from the Wellcome Sanger Institute were used as a replication cohort. The Cancer, Ageing and Somatic Mutation (CASM) pipeline was used to produce aligned BAM files, germline variants were called using bcftools mpileup and call. Low quality genotypes were hard filtered at GQ<20. External controls from gnomAD v2.1.1 exomes (34) (n=125,748) were used in ProxECAT (48) designed to do collapsing burden analysis with external controls. Using the InMeRF scoring criteria, one was predicted to be deleterious. However, given this is a replication analysis, and that InMeRF's particularly high specificity (>0.88) may likely be discounting many real deleterious variants, a CADD Phred score of >20 (i.e. the 1% most deleterious substitutions possible in the human genome) was used in the replication analysis, which yielded 4 deleterious non-synonymous variants. CADD and InMeRF show statistically similar predictions for the CTR9 gene, ICC=0.57 (p<0.05).

### PheWAS of CTR9

Phenotypes were mapped onto ICD10 codes using a Phecodes map file (49). Only phenotypes with more than n=60 cases were included in the analysis. 1027 phenotypes were tested. SAIGE-GENE was run on the CTR9 gene region with deleterious variants (as previously defined) individually on each phenotype and results combined.

### Somatic Mutation Analysis

Somatic calls in UKBB were derived from CRAM files using Mutect2. Quality control included excluding variants seen in a panel of normal samples (n=100) aged less than 40 years old, excluding variants with MMQ<25 and MBQ<15, excluding genotypes with a read depth (DP)<20 and a VAF<2%, as well as the default passing filters employed by Mutect2 and FilterMutectCalls. CHIP was defined in individuals according to previous standards using somatic calls (Table S2 in Bick et. al.) (6). Prevalence of CHIP in UKBB closely followed that of previous reports (FIG. 8). Within the 166,953 filtered samples used for this analysis, it was found that N_{chip}=4,682.

### Cell Culture, CRISPR Editing is CD34+HSPCs and HSC Phenotyping

HEK293T cells are cultured in DMEM supplemented with 10% FBS and 1× penicillin/streptomycin. Human CD34 HSPCs were purchased from Fred Hutchinson Cancer Institute and cultured in StemSpan serum free culture medium (StemCell Technologies) supplemented with 1×CC100, 2 mM L-glutamine, 100 ng/mL TPO, 1×UM171 and 1× penicillin/streptomycin. For RNP based CRISPR editing of CD34+HSPCs, chemically synthesized gRNA and Cas9 protein were mixed and set at room temperature for 10 mins to allow the formation of deliverable RNP. The assembled RNP complexes were then delivered into CD34+HSPCs by Lonza 4D Nucleofector with program DZ-100. 48-72 hrs after nucleofection, cells were harvested for genomic DNA and the edited region was PCR amplified and the editing efficiency was analyzed by the ICE analysis from Synthego. Edited CD34+HSPCs were allowed to expand in the culture medium and harvested twice at day 3 and day 6 post-nucleofection. Harvested cells were washed in cold PBS and stained by HSC phenotyping antibody cocktail, which is composed of Brilliant Violet 421 anti-CD34, APC-H7 anti-CD45RA, PE-Cy7 anti-CD90, BB700 anti-CD133, PE anti-EPCR and APC anti-ITGA3 in ice cold FACS buffer (PBS+2% FBS) at 1:60 for 30 minutes in ice. Stained cells were then analyzed by BD LSRII flow cytometer. For the chemical rescue experiment, CDK9 inhibitors (LDCO000067 and AZD4573) and the YEATS domain inhibitor of ENL/ILLT3 (SR-0813) were used at the concentration of IC50.

### Molecular Cloning and Co-Immunoprecipitation

Human CTR9 cDNA was amplified from the cDNA library of 293T cells. The amplified CTR9 cDNA was then TA cloned and sequenced for verification. pLVX-IRES-ZsGreen and pLVX-IRES-mCherry vector, which was modified from pLVX-IRES-ZsGreen by replacing ZsGreen by mCherry, was digested by NotI restriction enzyme at 37 C overnight. The verified CTR9 cDNA was amplified as three fragments with FLAG and/or HA tags included in the N terminus and overhangs overlapped each other and were brought into digested vectors by the InFusion cloning kit from ClonTech. Positive colonies were confirmed by Sanger Sequencing. All CTR9 mutation primers were designed by NEBase Changer online software (available on the world wide web at www.nebasechanger.neb.com) and the mutagenesis was performed using Q5 mutagenesis kit according to manufacturer's instructions. Sequence verified WT and mutant constructs were co-transfected by Lipofectamine 3000 kit at 1:1 molar ratio. For co-immunoprecipitation, cells were then harvested 48 hrs post-transfection (for HEK293T cells) or 5 days post-nucleofection (for CD34+HSPCs). The cells were then lysed by EBC buffer (50 mM Tris pH8.0, 120 mM NaCl, 0.5% NP40) in presence of protease inhibitor cocktail and PMSF. The extracted protein was then quantified by DC protein quantification assay and 0.5 mg of protein lysate was used for immunoprecipitation. Samples were pre-incubated with target antibody (PAF1c, AF9, FLAG, HA or IgG) for 2 hours at 4 C on a head-over-tail rotor, the protein-antibody complex was then incubated with 30 microliter of protein A/G dynabeads for addition 1 hour at 4 C. The supernatant was then removed and the beads were then washed by NETN buffer (20 mM Tris pH8.0, 100 mM NaCl, 0.5% NP40) for five times at room temperature. The immunoprecipitated proteins were then eluted by 1×SDS sample loading buffer with 55 C heat and run on 5%-20% precast gel along with 5% input for analysis.

### Colony Forming Unit Assay

Three days post-nucleofection, edited CD34+HSPCs were plated at a density of 500 cells/mL methylcellulose medium (H4034, Stem Cell Technologies) according to manufacturer's instructions. Plated cells were allowed to grow for 14 days before quantification and analysis.

### Single Cell RNA-Seq Analysis

Edited CD34+HSPCs were sorted and enriched for CD34+CD45RA-CD90+ population. The library was prepared using the 10×Genomics protocol according to the manufacturer's instructions. The sequencing results were pre-analyzed by the CellRanger pipeline to generate the matrix file, which was brought to downstream analysis. Normalization, scaling and cell clustering was done in R by the Seurat v4.0.2 package. The top 1000 most highly variable genes were used to calculate the top 50 principal components. The HSC cluster was highlighted by averaging the z-score normalized expression of CD34, HLF, CRHBP for each cell. The HSC population, defined by the averaged z score of more than 0.5 for the HSC signature, was used for DEG calling by DESeq2.

### Real Time PCR Analysis and Immunoblotting

RNA from HSPCs was harvested by Qiagen RNeasy kit and quantified by nanodrop. 1 microgram of total RNA was then reverse transcribed using Biorad iScript cDNA synthesis kit following manufacturer's instructions. The reverse transcribed cDNA was then diluted (1:20) and real time PCR was run using Biorad iQ SYBR green supermix. Data was normalized by loading control and presented as fold change compared to control samples. For immunoblotting, total protein lysate was extracted by RIPA buffer in presence of protease inhibitor cocktail and PMSF on ice for 10 minutes followed by a 5-minute incubation with MNase at 37 C. The total lysate was then linearized by 1×SDS loading buffer and heated at 55 C for 10 minutes. The lysate was run on 5-20% gradient gel and then transferred onto PVDF membrane. The membrane was then blocked by 5% nonfat dry milk in TBS/T. The membrane was then incubated with primary antibodies at 1:1000 dilution at 4 C overnight. HRP conjugated secondary antibodies were then incubated for 1 hr at room temperature. Membrane was developed using ECL and imaged in the Biorad gel imaging system.

### PRO-Seq Library Construction

Aliquots of frozen (−80° C.) permeabilized cells were thawed on ice and pipetted gently to fully resuspend. Aliquots were removed and permeabilized cells were counted using a Luna II, Logos Biosystems instrument. For each sample, 1 million permeabilized cells were used for nuclear run-on, with 50,000 permeabilized Drosophila S2 cells added to each sample for normalization. Nuclear run on assays and library preparation were performed essentially as described in Reimer et al. (53) [GSR1] with modifications noted: 2× nuclear run-on buffer consisted of (10 mM Tris 6 (pH 8), 10 mM MgCl2, 1 mM DTT, 300 mM KCl, 20 uM/ea biotin-11-NTPs (Perkin Elmer), 0.8 U/uL SuperaseIN (Thermo), 1% sarkosyl). Run-on reactions were performed at 37° C. Adenylated 3′ adapter was prepared using the 5′ DNA adenylation kit (NEB) and ligated using T4 RNA ligase 2, truncated KQ (NEB, per manufacturer's instructions with 15% PEG-8000 final) and incubated at 16° C. overnight. 180 μL of betaine blocking buffer (1.42 g of betaine brought to 10 mL with binding buffer supplemented to 0.6 uM blocking oligo (TCCGACGATCCCACGTTCCCGTGG/3InvdT/(SEQ ID NO: 14)) was mixed with ligations and incubated 5 min at 65° C. and 2 min on ice prior to addition of streptavidin beads. After T4 polynucleotide kinase (NEB) treatment, beads were washed once each with high salt, low salt, and blocking oligo wash (0.25×T4 RNA ligase buffer (NEB), 0.3 uM blocking oligo) solutions and resuspended in 5′ adapter mix (10 pmol 5′ adapter, 30 pmol blocking oligo, water). 5′ adapter ligation was per Reimer but with 15% PEG-8000 final. Eluted cDNA was amplified 5-cycles (NEBNext Ultra II Q5 master mix (NEB) with Illumina TruSeq PCR primers RP-1 and RPI-X) following the manufacturer's suggested cycling protocol for library construction. A portion of preCR was serially diluted and for test amplification to determine optimal amplification of final libraries. Pooled libraries were sequenced using the Illumina NovaSeq platform.

### PRO-Seq Data Analysis

All custom scripts described herein are available on the AdelmanLab Github (available on the world wide web at github.com/AdelmanLab/NIH_scripts). Using a custom script (trim_and_filter_PE.pl), FASTQ read pairs were trimmed to 41 bp per mate, and read pairs with a minimum average base quality score of 20 retained. Read pairs were further trimmed using cutadapt 1.14 to remove adapter sequences and low-quality 3′ bases (—match-read-wildcards −m 20−q 10). R1 reads, corresponding to RNA 3′ ends, were then aligned to the spiked in Drosophila genome index (dm6) using BWA, with those reads not mapping to the spike genome serving as input to the primary genome alignment step. Reads mapping to the hg38 reference genome were then sorted, via samtools 1.3.1 (-n), and subsequently converted to bam files. The bam files are converted to bigwig files by bamCoverage of deepTools-3.5. For metagene plots, bigwig files of three replicates of each group and combined and averaged using WiggleTools. The pausing indices were calculated using NRSA v2 packages (54).

### REFERENCES

1. A. Sud, S. Chattopadhyay, H. Thomsen, K. Sundquist, J. Sundquist, R. S. Houlston, K. Hemminki, Familial risks of acute myeloid leukemia, myelodysplastic syndromes, and myeloproliferative neoplasms. Blood. 132, 973-976 (2018).
2. E. L. Bao, S. K. Nandakumar, X. Liao, A. G. Bick, J. Karjalainen, M. Tabaka, O. I. Gan, A. S. Havulinna, T. T. J. Kiiskinen, C. A. Lareau, A. L. de Lapuente Portilla, B. Li, C. Emdin, V. Codd, C. P. Nelson, C. J. Walker, C. Churchhouse, A. de la Chapelle, D. E. Klein, B. Nilsson, P. W. F. Wilson, K. Cho, S. Pyarajan, J. M. Gaziano, N. J. Samani, FinnGen, 23andMe Research Team, A. Regev, A. Palotie, B. M. Neale, J. E. Dick, P. Natarajan, C. J. O'Donnell, M. J. Daly, M. Milyavsky, S. Kathiresan, V. G. Sankaran, Inherited myeloproliferative neoplasm risk affects haematopoietic stem cells. Nature. 586, 769-775 (2020).
3. C. J. Walker, C. C. Oakes, L. K. Genutis, B. Giacopelli, S. Liyanarachchi, D. Nicolet, A.-K. Eisfeld, M. Scholz, P. Brock, J. Kohlschmidt, K. Mrózek, M. Bill, A. J. Carroll, J. E. Kolitz, B. L. Powell, E. S. Wang, D. W. Niederwieser, R. M. Stone, J. C. Byrd, S. Schwind, A. de la Chapelle, C. D. Bloomfield, Genome-wide association study identifies an acute myeloid leukemia susceptibility locus near BICRA. Leukemia. 33, 771-775 (2019).
4. K. L. McGraw, C.-H. Cheng, Y. A. Chen, H.-A. Hou, B. Nilsson, G. Genovese, T. Cluzeau, A. Pellagatti, B. P. Przychodzen, M. Mallo, L. Arenillas, A. Mohamedali, L. Ades, D. A. Sallman, E. Padron, L. Sokol, C. Moreilhon, S. Raynaud, H.-F. Tien, J. Boultwood, B. L. Ebert, F. Sole, P. Fenaux, G. J. Mufti, J. P. Maciejewski, P. A. Kanetsky, A. F. List, Non-del(5q) myelodysplastic syndromes-associated loci detected by SNP-array genome-wide association meta-analysis. Blood Adv. 3, 3579-3589 (2019).
5. J. M. Klco, C. G. Mullighan, Advances in germline predisposition to acute leukaemias and myeloid neoplasms. Nat. Rev. Cancer. 21, 122-137 (2021).
6. G. Bick, J. S. Weinstock, S. K. Nandakumar, C. P. Fulco, E. L. Bao, S. M. Zekavat, M. Szeto, X. Liao, M. J. Leventhal, J. Nasser, K. Chang, C. Laurie, B. B. Burugula, C. J. Gibson, A. E. Lin, M. A. Taub, F. Aguet, K. Ardlie, B. D. Mitchell, K. C. Barnes, A. Moscati, M. Fornage, S. Redline, B. M. Psaty, E. K. Silverman, S. T. Weiss, N. D. Palmer, R. S. Vasan, E. G. Burchard, S. L. R. Kardia, J. He, R. C. Kaplan, N. L. Smith, D. K. Arnett, D. A. Schwartz, A. Correa, M. de Andrade, X. Guo, B. A. Konkle, B. Custer, J. M. Peralta, H. Gui, D. A. Meyers, S. T. McGarvey, I. Y.-D. Chen, M. B. Shoemaker, P. A. Peyser, J. G. Broome, S. M. Gogarten, F. F. Wang, Q. Wong, M. E. Montasser, M. Daya, E. Kenny, K. E. North, L. J. Launer, B. E. Cade, J. C. Bis, M. H. Cho, J. Lasky-Su, D. W. Bowden, L. A. Cupples, A. C. Y. Mak, L. C. Becker, J. A. Smith, T. N. Kelly, S. Aslibekyan, S. R. Heckbert, H. K. Tiwari, I. V. Yang, J. A. Heit, S. A. Lubitz, J. M. Johnsen, J. E. Curran, S. E. Wenzel, D. E. Weeks, D. C. Rao, D. Darbar, J.-Y. Moon, R. P. Tracy, E. J. Buth, N. Rafaels, R. J. F. Loos, P. Durda, Y. Liu, L. Hou, J. Lee, P. Kachroo, B. I. Freedman, D. Levy, L. F. Bielak, J. E. Hixson, J. S. Floyd, E. A. Whitsel, P. T. Ellinor, M. R. Irvin, T. E. Fingerlin, L. M. Raffield, S. M. Armasu, M. M. Wheeler, E. C. Sabino, J. Blangero, L. K. Williams, B. D. Levy, W. H.-H. Sheu, D. M. Roden, E. Boerwinkle, J. E. Manson, R. A. Mathias, P. Desai, K. D. Taylor, A. D. Johnson, NHLBI Trans-Omics for Precision Medicine Consortium, P. L. Auer, C. Kooperberg, C. C. Laurie, T. W. Blackwell, A. V. Smith, H. Zhao, E. Lange, L. Lange, S. S. Rich, J. I Rotter, J. G. Wilson, P. Scheet, J. O. Kitzman, E. S. Lander, J. M. Engreitz, B. L. Ebert, A. P. Reiner, S. Jaiswal, G. Abecasis, V. G. Sankaran, S. Kathiresan, P. Natarajan, Inherited causes of clonal haematopoiesis in 97,691 whole genomes. Nature. 586, 763-768 (2020).
7. R. L. Bowman, L. Busque, R. L. Levine, Clonal Hematopoiesis and Evolution to Hematopoietic Malignancies. Cell Stem Cell. 22, 157-170 (2018).
8. J. G. Tate, S. Bamford, H. C. Jubb, Z. Sondka, D. M. Beare, N. Bindal, H. Boutselakis, C. G. Cole, C. Creatore, E. Dawson, P. Fish, B. Harsha, C. Hathaway, S. C. Jupe, C. Y. Kok, K. Noble, L. Ponting, C. C. Ramshaw, C. E. Rye, H. E. Speedy, R. Stefancsik, S. L. Thompson, S. Wang, S. Ward, P. J. Campbell, S. A. Forbes, COSMIC: the Catalogue Of Somatic Mutations In Cancer. Nucleic Acids Res. 47, D941-D947 (2019).
9. M. Yu, W. Yang, T. Ni, Z. Tang, T. Nakadai, J. Zhu, R. G. Roeder, RNA polymerase II-associated factor 1 regulates the release and phosphorylation of paused RNA polymerase II. Science. 350, 1383-1386 (2015).
10. F. X. Chen, P. Xie, C. K. Collings, K. Cao, Y. Aoi, S. A. Marshall, E. J. Rendleman, M. Ugarenko, P. A. Ozark, A. Zhang, R. Shiekhattar, E. R. Smith, M. Q. Zhang, A. Shilatifard, PAF1 regulation of promoter-proximal pause release via enhancer activation. Science. 357, 1294-1298 (2017).
11. S. Hanks, E. R. Perdeaux, S. Seal, E. Ruark, S. S. Mahamdallie, A. Murray, E. Ramsay, S. Del Vecchio Duarte, A. Zachariou, B. de Souza, M. Warren-Perry, A. Elliott, A. Davidson, H. Price, C. Stiller, K. Pritchard-Jones, N. Rahman, Germline mutations in the PAF Icomplex gene CTR9 predispose to Wilms tumour. Nat. Commun. 5, 4398 (2014).
12. Y. Li, J. Zhang, P. R. Adikaram, J. Welch, B. Guan, L. S. Weinstein, H. Chen, W. F. Simonds, Genotype of CDC73 germline mutation determines risk of parathyroid cancer. Endocr. Relat. Cancer. 27, 483-494 (2020).
13. J. D. Carpten, C. M. Robbins, A. Villablanca, L. Forsberg, S. Presciuttini, J. Bailey-Wilson, W. F. Simonds, E. M. Gillanders, A. M. Kennedy, J. D. Chen, S. K. Agarwal, R. Sood, M. P. Jones, T. Y. Moses, C. Haven, D. Petillo, P. D. Leotlela, B. Harding, D. Cameron, A. A. Pannett, A. Höög, H. Heath 3rd, L. A. James-Newton, B. Robinson, R. J. Zarbo, B. M. Cavaco, W. Wassif, N. D. Perrier, I. B. Rosen, U. Kristoffersson, P. D. Turnpenny, L.-O. Farnebo, G. M. Besser, C. E. Jackson, H. Morreau, J. M. Trent, R. V. Thakker, S. J. Marx, B. T. Teh, C. Larsson, M. R. Hobbs, HRPT2, encoding parafibromin, is mutated in hyperparathyroidism-jaw tumor syndrome. Nat. Genet. 32, 676-680 (2002).
14. J. Grinfeld, J. Nangalia, E. J. Baxter, D. C. Wedge, N. Angelopoulos, R. Cantrill, A. L. Godfrey, E. Papaemmanuil, G. Gundem, C. MacLean, J. Cook, L. O'Neil, S. O'Meara, J. W. Teague, A. P. Butler, C. E. Massie, N. Williams, F. L. Nice, C. L. Andersen, H. C. Hasselbalch, P. Guglielmelli, M. F. McMullin, A. M. Vannucchi, C. N. Harrison, M. Gerstung, A. R. Green, P. J. Campbell, Classification and Personalized Prognosis in Myeloproliferative Neoplasms. N. Engl. J. Med. 379, 1416-1430 (2018).
15. S. M. Vos, L. Farnung, M. Boehning, C. Wigge, A. Linden, H. Urlaub, P. Cramer, Structure of activated transcription complex Pol II-DSIF-PAF-SPT6. Nature. 560, 607-612 (2018).
16. S. M. Vos, L. Farnung, A. Linden, H. Urlaub, P. Cramer, Structure of complete Pol II-DSIF-PAF-SPT6 transcription complex reveals RTF1 allosteric activation. Nat. Struct. Mol. Biol. 27, 668-677 (2020).
17. M. Yamashita, P. V. Dellorusso, O. C. Olson, E. Passegué, Dysregulated haematopoietic stem cell behaviour in myeloid leukaemogenesis. Nat. Rev. Cancer. 20, 365-382 (2020).
18. L. Cimmino, I. Dolgalev, Y. Wang, A. Yoshimi, G. H. Martin, J. Wang, V. Ng, B. Xia, M. T. Witkowski, M. Mitchell-Flack, I. Grillo, S. Bakogianni, D. Ndiaye-Lobry, M. T. Martin, M. Guillamot, R. S. Banh, M. Xu, M. E. Figueroa, R. A. Dickins, O. Abdel-Wahab, C. Y. Park, A. Tsirigos, B. G. Neel, I. Aifantis, Restoration of TET2 Function Blocks Aberrant Self-Renewal and Leukemia Progression. Cell. 170, 1079-1095.e20 (2017).
19. M. Agathocleous, C. E. Meacham, R. J. Burgess, E. Piskounova, Z. Zhao, G. M. Crane, B. L. Cowin, E. Bruner, M. M. Murphy, W. Chen, G. J. Spangrude, Z. Hu, R. J. DeBerardinis, S. J. Morrison, Ascorbate regulates haematopoietic stem cell function and leukaemogenesis. Nature. 549, 476-481 (2017).
20. E. Tomellini, I. Fares, B. Lehnertz, J. Chagraoui, N. Mayotte, T. MacRae, M.-È. Bordeleau, S. Corneau, R. Bisaillon, G. Sauvageau, Integrin-α3 Is a Functional Marker of Ex Vivo Expanded Human Long-Term Hematopoietic Stem Cells. Cell Rep. 28, 1063-1073.e5 (2019).
21. L. Wan, S. Chong, F. Xuan, A. Liang, X. Cui, L. Gates, T. S. Carroll, Y. Li, L. Feng, G. Chen, S.-P. Wang, M. V. Ortiz, S. K. Daley, X. Wang, H. Xuan, A. Kentsis, T. W. Muir, R. G. Roeder, H. Li, W. Li, R. Tjian, H. Wen, C. D. Allis, Impaired cell fate through gain-of-function mutations in a chromatin reader. Nature. 577, 121-126 (2020).
22. D. R. Dou, V. Calvanese, M. I. Sierra, A. T. Nguyen, A. Minasian, P. Saarikoski, R. Sasidharan, C. M. Ramirez, J. A. Zack, G. M. Crooks, Z. Galic, H. K. A. Mikkola, Medial HOXA genes demarcate haematopoietic stem cell fate during human development. Nat. Cell Biol. 18, 595-606 (2016).
23. N. Saha, J. Ropa, L. Chen, H. Hu, M. Mysliwski, A. Friedman, I. Maillard, A. G. Muntean, The PAF1c Subunit CDC73 Is Required for Mouse Hematopoietic Stem Cell Maintenance but Displays Leukemia-Specific Gene Regulation. Stem Cell Reports. 12, 1069-1083 (2019).
24. F. X. Chen, A. R. Woodfin, A. Gardini, R. A. Rickels, S. A. Marshall, E. R. Smith, R. Shiekhattar, A. Shilatifard, PAF1, a Molecular Regulator of Promoter-Proximal Pausing by RNA Polymerase II. Cell. 162, 1003-1015 (2015).
25. K. Hetzner, M.-P. Garcia-Cuellar, C. Buttner, R. K. Slany. The interaction of ENL with PAF1 mitigates polycomb silencing and facilitates murine leukemogenesis. Blood. 131, 662-673 (2018).
26. N. He, C. K. Chan, B. Sobhian, S. Chou, Y. Xue, M. Liu, T. Alber, M. Benkirane, Q. Zhou, Human Polymerase-Associated Factor complex (PAFc) connects the Super Elongation Complex (SEC) to RNA polymerase II on chromatin. Proc. Natl. Acad. Sci. U.S.A 108, E636-45 (2011).
27. V. Calvanese, A. T. Nguyen, T. J. Bolan, A. Vavilina, T. Su, L. K. Lee, Y. Wang, F. D. Lay, M. Magnusson, G. M. Crooks, S. K. Kurdistani, H. K. A. Mikkola, MLLT3 governs human haematopoietic stem-cell self-renewal and engraftment. Nature. 576, 281-286 (2019).
28. M. A. Erb, T. G. Scott, B. E. Li, H. Xie, J. Paulk, H.-S. Seo, A. Souza, J. M. Roberts, S. Dastjerdi, D. L. Buckley, N. E. Sanjana, O. Shalem, B. Nabet, R. Zeid, N. K. Offei-Addo, S. Dhe-Paganon, F. Zhang, S. H. Orkin, G. E. Winter, J. E. Bradner, Transcription control by the ENL YEATS domain in acute leukaemia. Nature. 543, 270-274 (2017).
29. L. Garnar-Wortzel, T. R. Bishop, S. Kitamura, N. Milosevich, J. N. Asiaban, X. Zhang, Q. Zheng, E. Chen, A. R. Ramos, C. J. Ackerman, E. N. Hampton, A. K. Chatterjee, T. S. Young, M. V. Hull, K. B. Sharpless, B. F. Cravatt, D. W. Wolan, M. A. Erb, Chemical Inhibition of ENL/AF9 YEATS Domains in Acute Leukemia. ACS Cent Sci. 7, 815-830 (2021).
30. L. Wan, H. Wen, Y. Li, J. Lyu, Y. Xi, T. Hoshii, J. K. Joseph, X. Wang, Y.-H. E. Loh, M. A. Erb, A. L. Souza, J. E. Bradner, L. Shen, W. Li, H. Li, C. D. Allis, S. A. Armstrong, X. Shi, ENL links histone acetylation to oncogenic gene expression in acute myeloid leukaemia. Nature. 543, 265-269 (2017).
31. J. Szustakowski, S. Balasubramanian, A. Sasson, S. Khalid, P. G. Bronson, E. Kvikstad, E. Wong, D. Liu, J. W. Davis, C. Haefliger, A. K. Loomis, R. Mikkilineni, H. J. Noh, S. Wadhawan, X. Bai, A. Hawes, O. Krasheninina, R. Ulloa, A. Lopez, E. N. Smith, J. Waring, C. D. Whelan, E. A. Tsai, J. Overton, W. Salerno, H. Jacob, S. Szalma, H. Runz, G. Hinkle, P. Nioi, S. Petrovski, M. R. Miller, A. Baras, L. Mitnaul, J. G. Reid, Advancing Human Genetics Research and Drug Discovery through Exome Sequencing of the UK Biobank. medRxiv, 2020.11.02.20222232 (2020).
32. Hail Team, Hail (available on the world wide web at github.com/hail-is/hail/commit/3c86d3ba497a).
33. W. McLaren, L. Gil, S. E. Hunt, H. S. Riat, G. R. S. Ritchie, A. Thormann, P. Flicek, F. Cunningham, The Ensembl Variant Effect Predictor. Genome Biol. 17, 122 (2016).
34. K. J. Karczewski, L. C. Francioli, G. Tiao, B. B. Cummings, J. Alfoldi, Q. Wang, R. L. Collins, K. M. Laricchia, A. Ganna, D. P. Birnbaum, L. D. Gauthier, H. Brand, M. Solomonson, N. A. Watts, D. Rhodes, M. Singer-Berk, E. M. England, E. G. Seaby, J. A. Kosmicki, R. K. Walters, K. Tashman, Y. Farjoun, E. Banks, T. Poterba, A. Wang, C. Seed, N. Whiffin, J. X. Chong, K. E. Samocha, E. Pierce-Hoffman, Z. Zappala, A. H. O'Donnell-Luria, E. V. Minikel, B. Weisburd, M. Lek, J. S. Ware, C. Vittal, I. M. Armean, L. Bergelson, K. Cibulskis, K. M. Connolly, M. Covarrubias, S. Donnelly, S. Ferriera, S. Gabriel, J. Gentry, N. Gupta, T. Jeandet, D. Kaplan, C. Llanwarne, R. Munshi, S. Novod, N. Petrillo, D. Roazen, V. Ruano-Rubio, A. Saltzman, M. Schleicher, J. Soto, K. Tibbetts, C. Tolonen, G. Wade, M. E. Talkowski, Genome Aggregation Database Consortium, B. M. Neale, M. J. Daly, D. G. MacArthur, The mutational constraint spectrum quantified from variation in 141,456 humans. Nature. 581, 434-443 (2020).
35. K. Wang, M. Li, H. Hakonarson, ANNOVAR: functional annotation of genetic variants from high-throughput sequencing data. Nucleic Acids Res. 38, e164 (2010).
36. X. Liu, C. Li, C. Mou, Y. Dong, Y. Tu, dbNSFP v4: a comprehensive database of transcript-specific functional predictions and annotations for human nonsynonymous and splice-site SNVs. Genome Med. 12, 103 (2020).
37. M. J. Landrum, J. M. Lee, M. Benson, G. R. Brown, C. Chao, S. Chitipiralla, B. Gu, J. Hart, D. Hoffman, W. Jang, K. Karapetyan, K. Katz, C. Liu, Z. Maddipatla, A. Malheiro, K. McDaniel, M. Ovetsky, G. Riley, G. Zhou, J. B. Holmes, B. L. Kattman, D. R. Maglott, ClinVar: improving access to variant interpretations and supporting evidence. Nucleic Acids Res. 46, D1062-D1067 (2018).
38. S. Balasubramanian, Y. Fu, M. Pawashe, P. McGillivray, M. Jin, J. Liu, K. J. Karczewski, D. G. MacArthur, M. Gerstein, Using ALoFT to determine the impact of putative loss-of-function variants in protein-coding genes. Nat. Commun. 8, 382 (2017).
39. J.-I. Takeda, K. Nanatsue, R. Yamagishi, M. Ito, N. Haga, H. Hirata, T. Ogi, K. Ohno, InMeRF: prediction of pathogenicity of missense variants by individual modeling for each amino acid substitution. NAR Genom Bioinform. 2, lqaa038 (2020).
40. W. Zhou, Z. Zhao, J. B. Nielsen, L. G. Fritsche, J. LeFaive, S. A. Gagliano Taliun, W. Bi, M. E. Gabrielsen, M. J. Daly, B. M. Neale, K. Hveem, G. R. Abecasis, C. J. Willer, S. Lee, Scalable generalized linear mixed model for region-based association tests in large biobanks and cohorts. bioRxiv, 583278 (2020).
41. C. C. Shaun Purcell, PLINK (available on the world wide web at www.cog-genomics.org/plink/2.0/).
42. C. C. Chang, C. C. Chow, L. C. Tellier, S. Vattikuti, S. M. Purcell, J. J. Lee, Second-generation PLINK: rising to the challenge of larger and richer datasets. Gigascience. 4, 7 (2015).
43. C. V. Van Hout, I. Tachmazidou, J. D. Backman, J. D. Hoffman, D. Liu, A. K. Pandey, C. Gonzaga-Jauregui, S. Khalid, B. Ye, N. Banerjee, A. H. Li, C. O'Dushlaine, A. Marcketta, J. Staples, C. Schurmann, A. Hawes, E. Maxwell, L. Barnard, A. Lopez, J. Penn, L. Habegger, A. L. Blumenfeld, X. Bai, S. O'Keeffe, A. Yadav, K. Praveen, M. Jones, W. J. Salerno, W. K. Chung, I. Surakka, C. J. Willer, K. Hveem, J. B. Leader, D. J. Carey, D. H. Ledbetter, Geisinger-Regeneron DiscovEHR Collaboration, L. Cardon, G. D. Yancopoulos, A. Economides, G. Coppola, A. R. Shuldiner, S. Balasubramanian, M. Cantor, Regeneron Genetics Center, M. R. Nelson, J. Whittaker, J. G. Reid, J. Marchini, J. D. Overton, R. A. Scott, G. R. Abecasis, L. Yerges-Armstrong, A. Baras, Exome sequencing and characterization of 49,960 individuals in the UK Biobank. Nature. 586, 749-756 (2020).
44. R Core Team, R: A Language and Environment for Statistical Computing (R Foundation for Statistical Computing, Vienna, Austria, 2020; available on the world wide web at www.R-project.org/).
45. H. Wickham, ggplot2: Elegant Graphics for Data Analysis (2016), (available on the world wide web at ggplot2.tidvverse.org).
46. Kosmidis, E. C. Kenne Pagui, N. Sartori, Mean and median bias reduction in generalized linear models. Stat. Comput. 30, 43-59 (2020).
47. B. Goodrich, J. Gabry, I. Ali, S. Brilleman, rstanarm: Bayesian applied regression modeling via Stan (2020), (available on the world wide web at mc-stan.org/rstanarm).
48. E. Hendricks, S. C. Billups, H. N. C. Pike, I. S. Farooqi, E. Zeggini, S. A. Santorico, I. Barroso, J. Dupuis, ProxECAT: Proxy External Controls Association Test. A new case-control gene region association test using allele frequencies from public controls. PLoS Genet. 14, e1007591 (2018).
49. P. Wu, A. Gifford, X. Meng, X. Li, H. Campbell, T. Varley, J. Zhao, L. Bastarache, J. C. Denny, E. Theodoratou, W.-Q. Wei, Developing and Evaluating Mappings of ICD-10 and ICD-10-CM codes to Phecodes. bioRxiv, 462077 (2018).

What is claimed:

1) A method for treating cancer, the method comprising administering to a subject having cancer an agent that inhibits the super elongation complex (SEC).

2) The methods of claim 1, wherein the cancer is a blood cancer.

3) The method of claim 2, wherein the blood cancer is selected from the group consisting of leukemia, lymphoma, myeloma, and myeloproliferative neoplasms (MPNs)

4) The method of claim 3, wherein the leukemia is selected from the group consisting of Acute myeloid leukemia (AML), Chronic myeloid leukemia (CML), Acute lymphocytic leukemia (ALL), and Chronic lymphocytic leukemia (CLL).
wherein the lymphoma is selected from the group consisting of a non-Hodgkin lymphoma, Hodgkin lymphoma, Diffuse large B-cell lymphoma (DLBCL), Follicular lymphoma, Chronic lymphocytic leukemia (CLL), Small lymphocytic lymphoma (SLL), Mantle cell lymphoma (MCL), Marginal zone lymphomas, and Burkitt lymphoma,
wherein the myeloma is selected from the group consisting of multiple myeloma, plasmacytoma, and monoclonal gammopathy of undetermined significance (MGUS), or
wherein the MPN is selected from the group consisting of Polycythemia vera (PV), Essential thrombocythemia (ET), and Myelofibrosis (MF).

5)-7) (canceled)

8) The method of claim 1, wherein the cancer results from or comprises a mutation that impacts hematopoietic stem cell self-renewal.

9) The method of claim 1, wherein the cancer results from or comprises a mutation in the CTR9 gene or PAF1 complex.

10) (canceled)

11) The method of claim 1, wherein the agent inhibits at least one component of the super elongation complex (SEC).

12) The method of claim 11, wherein the components of the SEC include Eleven-nineteen lysine-rich leukemia (ELL) 1, ELL2, ELL3, ELL-associated factor (EAF) 1/, EAF2, Mixed-lineage leukemia translocated to chromosome 3 (MLLT3) protein AF-9 (AF9), Mixed-lineage leukemia translocated to chromosome 1 (MLLT1) protein ENL, AF4/FMR2 Family member (AFF) 1, AFF4, and positive transcription elongation factor (P-TEFb).

13) The method of claim 12 wherein the P-TEFb component is selected from the group consisting of cyclin-dependent kinase 9 (CDK9), cyclin T (CycT) 1, CycT2, CycT3, bromodomain containing 4 protein (BRD4), and 7SK snRNP.

14) The method of claim 1, wherein the agent that inhibits the SEC is selected from the group consisting of a small molecule inhibitor, a small molecule degrader (proteolysis-targeting chimera, PROTAC), an antibody, a peptide, a genome editing system, an antisense oligonucleotide, and an RNAi.

15) The method of claim 14, wherein the small molecule is selected from the group consisting of: SR-0813, LDC000067, AZD4573, atuveciclib, flavopiridol, CR8, indirubin-3′-monoxime derivatives, 5-fluoro-N²,N⁴-diphenylpyrimidine-2,4-diamines, 4-(thiazol-5-ul)-2-(phenylamino)pyrimidines, TG02, CDKI-73, 2,4,5-trisubstited pyrimidine derivatives, Wogonin, PHA-767491, LY2857785, dinaciclib, roscovitine, voruciclib, sns-032, P276-00, FIT-039, CCT068127, MC180295, and KL-1, SR-1114, dTAG13, A-1592668.

16) (canceled)

17) (canceled)

18) The method of claim 1, wherein the subject has previously been administered an anti-cancer therapy.

19) The method of claim 1, wherein the subject has not previously been administered an anti-cancer therapy.

20) The method of claim 1, further comprising the step of, after the step of administering, administering at least one anti-cancer therapy to the subject.

21) The method of claim 1, further comprising the step of, prior to the step of administering, administering at least one anti-cancer therapy to the subject.

22) The method of claim 1, further comprising the step of, after the step of administering, administering low dose chemotherapy to the subject.

23) The method of claim 1, further comprising the step of, prior to administering, diagnosing a subject as having cancer or receiving a result from an assay that diagnoses a subject as having cancer.

24) (canceled)

25) The method of claim 18, wherein the anti-cancer treatment is selected from the list consisting of: chemotherapy, hematopoietic stem cell transplant, radiation, chemo-radiation, surgery, chimeric antigen receptor T-cells, immune checkpoint inhibitor, an antibody targeting an antigen on cancer cells, a toxin-conjugated antibody targeting an antigen on cancer cells, and a bispecific antibody that recruits a normal immune cell to a tumor cell by simultaneously binding antigens expressed on each of these cells.

26) A composition comprising an agent that inhibits the SEC.

27) The composition of claim 26, wherein the agent is a small molecule inhibitor, a small molecule degrader (proteolysis-targeting chimera, PROTAC), an antibody, a peptide, a genome editing system, an antisense oligonucleotide, and an RNAi

28) (canceled)

29) (canceled)
